(19) United States
(12) Patent Application Publication
(10) Pub. No.: US 2025/0110129 A1
(43) Pub. Date: Apr. 3, 2025

(54) THERMALLY RESPONSIVE PARTITIONS FOR DEVICES AND SYSTEMS AND METHODS OF USING SAME

(71) Applicant: University of Maryland, College Park, College Park, MD (US)

(72) Inventors: Ian M. WHITE, Washington, DC (US); Micaela EVERITT, Washington, DC (US); David BOEGNER, White Hall, MD (US); John Robertson RZASA, University Park, MD (US); Rachel WARREN, Silver Spring, MD (US)

(21) Appl. No.: 18/900,552

(22) Filed: Sep. 27, 2024

Related U.S. Application Data
(60) Provisional application No. 63/586,399, filed on Sep. 28, 2023.
(60) Provisional application No. 63/657,039, filed on Jun. 6, 2024.
(60) Provisional application No. 63/666,095, filed on Jun. 28, 2024.

(51) Int. Cl. G01N 33/569; G01N 21/64; G01N 33/543

(52) U.S. Cl. CPC G01N 33/56983; G01N 21/6428; G01N 33/54333; G01N 2021/6439; G01N 2333/165; G01N 2333/958; G01N 2469/20

(57) ABSTRACT

A method and test system for detecting the presence of an analyte in a sample comprising a binding region and a detecting region, where the binding region contains a plurality of magnetic beads attached to a plurality of first capture molecules that bind to an analyte of interest in the sample, and a plurality of second capture molecules having a detectable label attached thereto, where the second capture molecules bind to the analyte of interest to form a complex, where the complexes are moved through at least one liquefied aliphatic partition via a magnetic field into the detecting region that having a detection composition allows for detection and, optionally, for signal quantification.

### CROSS REFERENCE TO RELATED APPLICATIONS

This application claims the benefit of priority to U.S. Provisional Application No. 63/586,399, filed on Sep. 28, 2023, U.S. Provisional Application No. 63/657,039, filed on Jun. 6, 2024, and U.S. Provisional Application No. 63/666,095, filed on Jun. 28, 2024, the disclosures of which are incorporated herein by reference.

### STATEMENT REGARDING FEDERALLY SPONSORED RESEARCH

This invention was made with government support under R01 GM130923 and R61 HL167252 awarded by the National Institutes of Health. The government has certain rights in the invention.

### SEQUENCE LISTING

The instant application contains a Sequence Listing, which has been submitted in .xml format and is hereby incorporated by reference in its entirety. Said .xml copy was created on Sep. 27, 2024, is named “070919_00151_ST26.xml”, and is 7,377 bytes in size.

### BACKGROUND OF THE DISCLOSURE

Despite decades of research, the commercial impact of point-of-care (PoC) diagnostics has been minimal, and the need for global health diagnostics remains unaddressed. The lack of translation from research labs to the field may be due to incorrect assumptions and prioritizations about the requirements for use at the point of care. While efforts to shrink sensors or automate functions through microfluidics aim to satisfy some of the requirements outlined by the World Health Organization's “ASSURED” criteria, often the user-friendly (U in ASSURED) point is overlooked, despite its importance. In the United States, the Food and Drug Administration requires that diagnostic tests seeking approval for use outside of the central lab (i.e., to receive a Clinical Laboratory Improvement Amendment (CLIA) waiver) must be performed without the need for precise manual steps or interventions. This implies that diagnostic solutions should be sample-to-answer (S2A) to enable deployment beyond the central lab. To be a viable S2A diagnostic, a system must be able to process raw samples (including whole blood), enable precise reagent transfer and mixing, and perform rinses, all without manual interventions, bulky robotics, or complex valve controls.

Today, one common method for PoC protein biomarker detection is the lateral flow immunoassay (LFIA). All reagents are incorporated and stabilized, implying that precise reagent transfers are not necessary. Furthermore, the wicking of the membrane enables automated sample movement without the need for fluidics equipment. When the biomarker is present, a sandwich assay forms at a specific location on a paper strip, producing a visible line. As LFIAs require no wash steps, they are significantly faster and more hands-off than their gold standard counterparts, enzyme-linked immunosorbent assays (ELISAs). Saliva-based LFIA tests for HIV were an initial commercial success, and multiple nasal-swab-based COVID-19 tests quickly emerged in 2020, further proving the technology. This technology has been developed into diagnostic assays to rapidly diagnose viruses in serum or virus transport medium such as ebolavirus and influenza. While serum or saliva samples can be applied directly to the LFIA, complex raw samples, like whole blood, require preparation before the test can be performed. Although some progress has been made to separate plasma from whole blood by integrating a membrane-based separator into the LFIA, there are no commercially available LFIAs for protein detection in whole blood that are S2A when using complex raw samples.

The field of microfluidics has long pursued point-of-care diagnostics under the premise that precise reagent transfer steps can be automated by microfluidics, while biosensors can be integrated into the microsystems. Several reports demonstrate a reduction in the number of steps through non-chemical-based bacterial/viral lysis methods, and alternative DNA/RNA purification methods. While a reduction in the number of steps represents progress, these approaches still require one or more precise reagent transfers.

A few microfluidic methods have demonstrated S2A capabilities, but these methods have downfalls, including the need for cost-prohibitive chips or external pumps with manual tubing exchanges.

Magnetofluidics has recently emerged as a means to automate sample preparation, and thus to provide hands-off assay operation in PoC diagnostics. In initial reports, ferrofluids were manipulated with magnets underneath the device surface to transport droplets of reagents along the top of a surface, enabling automatic sample preparation for nucleic acid amplification tests. The surface of the nanoparticles serves to bind molecules within a droplet (e.g., DNA from cell lysate) and transport them to other droplets of reagents (e.g. rinsing and elution solutions). While these implementations pioneered a new hands-free method of sample manipulation, the reagents are typically not enclosed and are thus susceptible to contamination, evaporation, and mechanical disturbance.

Therefore, new methods and devices for point of care diagnostics are needed. These new methods and devices can allow for the detection of an analyte within a raw sample through a sample to answer immunoassay that is low cost and does not require bulky equipment.

### SUMMARY OF THE DISCLOSURE

The present disclosure provides a method and device utilizing thermally responsive aliphatic partitions (TRAPS) that initially separate reagent mixtures and continue to separate reagent mixtures, when liquefied, to allow magnetic beads to be pulled through the liquefied partitions in a magnetofluidics assay.

Various examples of the present disclosure provide a method for detecting the presence of an analyte. The method may comprise contacting a sample suspected of containing the analyte together or separately with (1) a plurality of magnetic beads that have a plurality of first capture molecules having specific affinity for the analyte attached thereto, and (2) a plurality of second capture molecules having a detectable label attached thereto and the second capture molecule has a specific affinity for the analyte, in order to form complexes comprising the analyte bound to the first capture molecule attached to the magnetic bead and the second capture molecule bound to the analyte. This method further comprises separating the complexes from unbound second capture molecules by selectively moving the complexes through one or more aliphatic partitions via application of a magnetic field, wherein the aliphatic partition has a melting point from 40° C. to 65° C. This method may further comprise detecting and optionally quantifying the signal generated from the detectable labels of the second capture molecules of the separated complexes. The presence of the detectable signal may be indicative of the presence of the analyte and the magnitude of the signal may be indicative of the amount of the analyte in the sample.

According to various examples of the present disclosure, the sample may be a biological sample.

According to various examples of the present disclosure, the sample may be chosen from whole blood, blood fractions, plasma, serum, saliva, urine, stool, sweat, mucous, tears, breast milk, semen, tissue, placental tissue, conditioned medium, tissue culture medium, and bone marrow.

According to various examples of the present disclosure, the analyte may be an antibody or an antigen.

According to various examples of the present disclosure, the antibody may be directed to a pathogenic antigen (e.g., a microbial antigen, bacterial antigen, or viral antigen).

According to various examples of the present disclosure, a viral antigen may be associated with SARS-COV-2, Hepatitis C, Epstein-Barr, Zika, Ebola, Herpes simplex, Norovirus, Influenza, or Chikungunya.

According to various examples of the present disclosure, the plurality of magnetic beads may have a longest linear dimension of 100 nm to 50,000 nm.

According to various examples of the present disclosure, the second capture molecule have horseradish peroxidase attached thereto.

According to various examples of the present disclosure, the detection composition may comprise a detection molecule that may be colorimetric, luminescent, or fluorescent.

According to various examples of the present disclosure, a colorimetric detection molecule may be 3,3′,5,5′-Tetramethylbenzidine (TMB).

According to various examples of the present disclosure, a luminescent detection molecule may be Luminol.

According to various examples of the present disclosure, a fluorescent detection molecule may be Amplex Red.

According to various examples of the present disclosure, the sample may be sequentially contacted with the plurality of magnetic beads and the plurality of second capture molecules.

According to various examples of the present disclosure, the sample may be rinsed after contacting the plurality of magnetic beads and prior to contacting the plurality of second capture molecules.

According to various examples of the present disclosure, the aliphatic partition may comprise one or more alkanes.

According to various examples of the present disclosure, one or more alkanes may be chosen from eicosane, docosane, hexacosane, heptacosane, nonococane, tetracosane, and octadecane.

According to various examples of the present disclosure, the magnetic field may be generated by a magnet.

According to various examples of the present disclosure, the magnet may be moving at a rate of 0.2 mm/s to 10 mm/s.

According to an example of the present disclosure the detectable signal may be colorimetric or fluorescent.

An aspect of the present disclosure is a testing system. The testing system may comprise a test assembly. The test assembly may comprise an inlet configured to accept a sample, a plurality of regions, and at least one aliphatic partition disposed therein. Adjacent regions of the plurality of regions may be separated by at least one of the aliphatic partitions. The plurality of regions may include a binding region and a detecting region. The binding region may comprise a plurality of magnetic beads having a plurality of first capture molecules attached thereto, wherein the first capture molecules have a specific affinity for the analyte and a plurality of second capture molecules having a detectable label attached thereto, wherein the second capture molecule has a specific affinity for the analyte. The detecting region may comprise a detection composition that allows direct or indirect spectrophotometric measurement of the detectable label. The testing system may comprise a magnet configured to apply a magnetic field along a sequential path that may include the binding region and the detecting region. The plurality of magnetic beads may move sequentially along the sequential path upon application of a magnetic force from the magnet.

According to various examples of the present disclosure, the sample may be an antibody or an antigen.

According to various examples of the present disclosure, the antibody may be associated with SARS-COV-2, Hepatitis C, Epstein-Barr, Zika, Ebola, Herpes simplex, Norovirus, Influenza, or Chikungunya.

According to various examples of the present disclosure, the aliphatic partition may comprise one or more alkanes.

According to various examples of the present disclosure, one or more alkanes may be chosen from eicosane, docosane, hexacosane, heptacosane, nonococane, tetracosane, and octadecane.

According to various examples of the present disclosure, at least one aliphatic partition, the binding region, and the detecting region may be arranged in a horizontal hydrophobic channel or a vertical hydrophobic channel.

According to various examples of the present disclosure, at least one aliphatic partition, the binding region, and the detecting region may be arranged in a horizontal hydrophilic channel or a vertical hydrophilic channel.

According to various examples of the present disclosure, at least one of the aliphatic partitions may be configured to separate adjacent regions/sub-regions (e.g., the binding region and the detecting region).

According to various examples of the present disclosure, the binding region may comprise a plurality of sub-regions, and each adjacent region/sub-region may be separated by at least one aliphatic partition.

According to various examples of the present disclosure, the plurality of sub-regions may comprise a first binding sub-region and a second binding sub-region.

According to various examples of the present disclosure, the first binding sub-region may comprise the plurality of magnetic beads attached to a plurality of first capture molecules having a specific affinity for the analyte.

According to various examples of the present disclosure, the second binding sub-region may comprise the plurality of second capture molecules attached to a detectable label where the second capture molecules have a specific affinity for the analyte.

According to various examples of the present disclosure, the test assembly may comprise a rinsing sub-region disposed between the aliphatic partition of the first binding sub-region and the aliphatic partition of the second binding sub-region.

According to various examples of the present disclosure, the magnet may be external to the test assembly.

According to various examples of the present disclosure, the magnet may be configured to move across the test assembly along the sequential path.

According to various examples of the present disclosure, the test assembly may move across a sequential path relative to a magnet.

According to various examples of the present disclosure, the magnet may be disposed on an end of the test assembly and the magnetic field may extend across the binding region and the detecting region sequentially.

The present disclosure provides a handheld measurement device with an assay method using one or more thermally-responsive aliphatic partitions (“TRAP(s)”) that can quickly and accurately test samples—e.g., test for the presence of one or more analytes in a sample. In one example, a device of the present disclosure detects the presence of histones in blood for point of care diagnostics at a medical facility to determine if someone is at risk for developing trauma-induced multiple organ failure in near-patient settings.

The device is small, may be battery powered, and is easy to use—for example, having only a single button to start a measurement and a multicolor LED to indicate the result. As of now, there is no established commercial standard for determining organ failure, although some hospitals utilize prediction models. These models, however, do not provide accurate patient diagnoses; instead, they predict mortality rates based on patient history. Given that most deaths resulting from severe traumatic injuries occur within the first 24 hours, it is evident that an accurate and portable diagnostic device to diagnose a patient as going into multiple organ failure is needed. An example of the present device addresses this need by aiming to detect circulating histones in the blood in which elevated levels of histones have been correlated to multiple organ failures.

In another example, the device may be used to quickly and accurately measure the presence of a virus in blood for point of care diagnostics at a medical facility. For example, the device can be used to detect SARS-COV-2. Examples of the device may be used to detect other viruses with appropriate tuning of the assay chemistry. While there are other laboratory-grade instruments that can give a standard of care measurement, they are often expensive, require a skilled user, and have time consuming testing procedures with multiple manual steps. Examples of the present device only require a user to insert a sample and it will provide a result in a short time (e.g., within an hour).

### BRIEF DESCRIPTION OF THE FIGURES

For a fuller understanding of the nature and objects of the disclosure, reference should be made to the following detailed description taken in conjunction with the accompanying figures.

FIG. 1 shows a cartoon representation of a method example for detecting the presence of an analyte, utilizing three distinct compartments and two aliphatic partitions.

FIG. 2 shows a diagram representation of a method for detecting the presence of an analyte.

FIG. 3 shows a schematic of sample-to-answer immunoassay from whole blood. The first zone houses magnetic beads functionalized with SARS-COV-2 spike proteins that capture antibodies against the SARS-COV-2 spike protein in the whole blood sample. As the cartridge is heated, the TRAPs liquefy, allowing an external magnet to pull the beads through the first TRAP to the second zone. HRP-conjugated secondary antibodies in this second zone label any antibodies captured by the bead. These complexes are pulled through rinses to shed any labels that are caught in the hydration layer of the beads before reaching the detection zone. The detection zone contains Amplex Red and hydrogen peroxide, which fluoresces in the presence of HRP.

FIG. 4 shows a top perspective view of a test assembly.

FIG. 5 shows photos of demonstration of capillary-based cap for automated blood collection. (A) First the capillary cap is placed on the cartridge, then (B) the capillary is placed in contact with the blood sample (e.g., droplet on a fingertip) and (C) blood is quickly (i.e. 60 seconds) wicked into the capillary until (D) a precise volume is reached. (E) The cartridge is tapped vertically to dispel blood into the cartridge and (F) the capillary top is replaced with the sealed top. A video of a whole blood sample being loaded into the cartridge can be viewed in the supplement.

FIG. 6 shows (A) 3D representation of a device with an alkane TRAP partitioning two liquids. (B) Hypothetical immunoassay using TRAPS. Alkane wax layers partition all reagents. When warmed, the partitions liquefy, enabling magnetic beads to be pulled from one reagent zone to the next, enabling sample-to-answer assays.

FIG. 7 shows a TRAP with yellow dyed water on top of eicosane on top of blue dyed water.

FIG. 8 shows a schematic illustration of a portable fluorescence reader.

FIG. 9 shows (A) a photograph of a melted TRAP in a vertical hydrophobic channel. (B) 2D schematic of a cross-section along the center of the channel parallel to the image plane in (A) before and after melting the TRAP. (C) Data showing TRAPs that remained intact (circle) and TRAPs that breached (triangles) as well as the threshold (dashed line) determined by a mathematical approximation.

FIG. 10 shows (A) a photograph of a melted TRAP in a horizontal hydrophobic channel. (B) 2D schematic of a cross-section along the center of the channel parallel to the image plane in (A) before and after melting the TRAP. (C) Data showing TRAPs that remained intact (circle) and TRAPs that breached (triangles) as well as the threshold (dashed line) determined by the mathematical approximation from the vertical hydrophobic case.

FIG. 11 shows (A) a photograph of a melted TRAP in a vertical hydrophilic channel. (B) 2D schematic of a cross-section along the center of the channel parallel to the image plane in (A) before and after melting the TRAP. (C) Data showing TRAPs that remained intact (circle) and TRAPs that breached (triangles) as well as the threshold (dashed line) determined by a mathematical approximation

FIG. 12 shows (A) a photograph of a melted TRAP in a horizontal hydrophilic channel. (B) 2D schematic of a cross-section along the center of the channel parallel to the image plane in (A) before and after melting the TRAP. (C) Data showing TRAPs that remained intact (green circle) and TRAPs that breached (red triangles) as well as the threshold (dashed line) determined by a mathematical approximation.

FIG. 13 shows (A) a schematic of the experiment in which a fluorescence measurement was taken before and after magnetic beads were transferred across a TRAP. (B) Data showing fluorescence values of the FAM side (green) and water side (blue) of the TRAP.

FIG. 14 shows (A) a schematic depicting that fewer magnetic beads and a thicker TRAP (top) will not bridge while more beads and a thinner TRAP (bottom) may bridge, causing the two liquids to mix. (B) Data showing which experimental combinations of plug length and bead mass caused TRAPs to remain intact (green circle) or bridge (red triangle). (C) Before (top) and after (bottom) photos of 64 μg of beads travelling through a 2 mm plug length with no TRAP bridging.

FIG. 15 shows fluorescence signals of antibody-bound beads that travelled through one, two, or three TRAPs as well as a no target control (NTC) which included beads with no antibodies that travelled through one TRAP. n=3.

FIG. 16 shows impact of wax and heat on antibody sandwiches. (N=3) (A) Fluorescence measured before and after pulling beads across liquefied alkane partition. (B) Antibody sandwiches were exposed to 60° C. to determine heat impact on stability.

FIG. 17 shows (A) removal of biomarker from blood. Spike protein antibody is captured on magnetic bead and pulled across liquefied alkane to produce fluorescence. (N=3). (B) detection of SARS-COV-2 antibodies using the sample-to-answer platform. (N=3).

FIG. 18 shows a schematic of a portable fluorescence reader with built in heater. (A) The ArduCAM, LEDs, filter, and heater are housed in a 3D printed handheld device. (B) The device opens to separate the heater from the ArduCAM to insert an assay cartridge. (C) The ArduCAM is housed behind a longpass filter to detect fluorescence produced from a sample, which is excited by LEDs built into the device above the sample cartridge. The cartridge is placed on top of a polyimide heater to liquefy the TRAPs.

FIG. 19 shows photos of magnetic beads pulled through TRAPs in cartridge. The cartridge is heated to melt the TRAPs. Once liquefied, the magnetic beads are pulled through each of the permeable barriers. The cartridge is removed from heat and the TRAPs harden. A video of magnetic beads being pulled through TRAPs can be viewed in the supplement.

FIG. 20 shows fluorescence quantification of antibodies against SARS-COV-2 spike protein in whole blood over 14 minutes. The heater is turned off at t=0 minutes, right after the magnetic beads reach the detection zone.

FIG. 21 shows (A) fluorescence quantification of antibodies against SARS-CoV-2 spike protein in whole blood using our sample-to-answer immunoassay and portable fluorescence reader (N=3). The detection limit is 84 μg/mL using the IUPAC definition. Error bars are +/−1 standard deviation. (B) Fluorescence quantification of antibodies against SARS-COV-2 spike protein in whole blood using a bead-based immunoassay with manual rinse steps and benchtop plate reader (N=3). The detection limit is 68 μg/mL using the IUPAC definition. Error bars are +/−1 standard deviation.

FIG. 22 shows (A) application of the TRAP in which magnetic beads move biomarkers out of a blood layer through a melted (liquefied) alkane into a rinse zone. (B) Photo and schematic of a resin channel showing two dyed water layers separated by a TRAP.

FIG. 23 shows several combinations of plug length and channel width for vertical (left) and horizontal (right), hydrophobic (top), hydrophilic (bottom) channels. Circles=TRAP stayed, triangle=TRAP broke and dyes mixed.

FIG. 24 shows fluorescence measurements of the water layer (bottom marks on left plot) and fluorescence layer (top marks on left plot) along with their corresponding schematics of a channel before and after beads were pulled through a TRAP via a magnet.

FIG. 25 shows a carton depiction of TRAP systems of the present disclosure. On the left is an example of a system used to detect DNA/RNA in a sample (e.g., wastewater) and on the right is a system used to detect a virus from a complex sample (e.g., blood).

FIG. 26 shows a picture of a TRAP system used to detect a virus from blood.

FIG. 27 shows a schematic of the mechanisms within a cassette during the diagnostic test. After a blood sample is loaded into the cassette, magnetic microbeads functionalized with aptamers capture virus particles present in the sample. The temperature is increased to melt the eicosane layer, allowing a magnet to pull beads through, isolating virus particles from the blood sample. Once through, the virus particles are lysed. After lysis, the hexacosane layer is melted, initiating the LAMP reaction.

FIG. 28 shows a photo and schematic of prototype device hardware. The photo includes a penny for scale. This device is capable of testing four samples at one time.

FIG. 29 shows (A) photos depicting each stage of the assay within a cassette with dyes in each solution. Magnetic beads traverse the eicosane stationary TRAP without red dye leaking through. When the hexacosane removable TRAP melts, the layers on either side quickly mix. (B) Data representing the speed at which two liquids mix upon melting a removable TRAP.

FIG. 30 shows LAMP results from a sample of virus particles after an incubation with Proteinase K (orange curve) compared to a sample of virus particles without a Proteinase K incubation (green curve), along with a no-target control (blue curve).

FIG. 31 shows LAMP results from a sample of virus particles after capture onto magnetic beads and transfer across a melted eicosane TRAP (orange curve) compared to transfer across an oil partition at room temperature (green curve), along with a no-target control (blue curve).

FIG. 32 shows (A) fluorescence data from a portable reader device plotted on an annotated graph that includes the temperature profile inside the device (dotted line). (B) Compiled results from 35 blood tests using our device.

FIG. 33 shows COVID-19 patient plasma samples tested via RT-qPCR (x-axis) and via our S2A device (y-axis). Samples confirmed positive by RT-qPCR are colored orange while those not detected by RT-qPCR are colored blue.

FIG. 34 shows (A) fluorescence data resulting from PCR of several concentrations of SARS-COV-2. The threshold used to quantify Ct values is also plotted (dotted line). (B) A first degree best fit curve is generated and displayed to correlate concentrations with Ct values and to provide an ability to estimate virus concentrations within the patient samples.

FIG. 35 shows a schematic of the mechanisms within a cassette during the diagnostic test. After a blood sample is loaded into the cassette, magnetic microbeads functionalized with aptamers capture virus particles present in the sample. The temperature is increased to melt the eicosane layer, allowing a magnet to pull beads through, isolating virus particles from the blood sample. Once through, the virus particles are lysed. After lysis, the hexacosane layer is melted, initiating the LAMP reaction.

FIG. 36 shows a photo and schematic of prototype device hardware. The photo includes a penny for scale. This device is capable of testing four samples at one time.

FIG. 37 shows (A) photos depicting each stage of the assay within a cassette with dyes in each solution. Magnetic beads traverse the eicosane stationary TRAP without red dye leaking through. When the hexacosane removable TRAP melts, the layers on either side quickly mix. (B) Data representing the speed at which two liquids mix upon melting a removable TRAP.

FIG. 38 shows LAMP results from a sample of virus particles after an incubation with Proteinase K (orange curve) compared to a sample of virus particles without a Proteinase K incubation (green curve), along with a no-target control (blue curve).

FIG. 39 shows LAMP results from a sample of virus particles after capture onto magnetic beads and transfer across a melted eicosane TRAP (orange curve) compared to transfer across an oil partition at room temperature (green curve), along with a no-target control (blue curve).

FIG. 40 shows (A) fluorescence data from our portable reader device plotted on an annotated graph that includes the temperature profile inside the device (dotted line). (B) Compiled results from 35 blood tests using our device.

FIG. 41 shows COVID-19 patient plasma samples tested via RT-qPCR (x-axis) and via our S2A device (y-axis). Samples confirmed positive by RT-qPCR are colored orange while those not detected by RT-qPCR are colored blue.

FIG. 42 shows a sample cartridge detail for another example of the present disclosure (the cartridge used in the PHAST prototype).

FIG. 43 shows a block diagram of PHAST platform.

FIG. 44A shows a CAD model of a device according to an example of the present disclosure without lid removed.

FIG. 44B shows a CAD model of the device of FIG. 37A shown with the lid in place.

FIG. 45 shows a photograph of a prototype device according to an example of the present disclosure.

FIG. 46 shows an example screen of configuration control software for the PHAST platform.

FIG. 47 shows a sample cartridge detail for another example of the present disclosure (the cartridge used in the STAT prototype).

FIG. 48 shows a chart showing a temperature profile (i.e., testing profile) of a STAT example.

FIG. 49 shows a block diagram of STAT platform.

FIG. 50 shows an example screen of configuration control software for the STAT platform.

FIG. 51 shows a schematic of the saliva-STAT diagnostic test for the SARS-CoV-2 virus. After a saliva sample is deposited into the funnel attached to a cassette, magnetic microbeads functionalized with aptamers capture virus particles present in the sample. The temperature is increased to melt the eicosane layer, allowing a magnet to pull beads through, isolating virus particles from the saliva sample. Once through, the virus particles are lysed. After lysis, the hexacosane layer is melted, initiating the LAMP reaction.

FIG. 52 shows a (A) rendering of the assembled saliva-STAT handheld instrument and exploded view. (B) Photo of the instrument with a sample cassette.

FIG. 53 shows images of a cassette with a funnel attachment as paramagnetic beads are pulled downward in a saliva sample. The data represents the bead transfer from the saliva sample to the saliva-TRAP interface. The red color value was extracted from a small window near the top of the cassette.

FIG. 54 shows a (A) fluorescence readout of the bottom region in a cassette during a hexacosane melt, which causes intercalating fluorescent dye to mix with a solution of DNA. (B) The temperature profile shows that the hexacosane breaches within one minute of when the cassette reaches the melting point (˜57° C.).

FIG. 55 shows LAMP in the saliva-STAT. (A) Representative amplification curves for 500 copies and 0 copies of viral RNA in the cassette under a layer of eicosane, heated and measured in the saliva-STAT instrument. (B) Mean times-to-positive for 0, 5, 50, and 500 copies of RNA in the saliva-STAT cassette under a layer of eicosane, heated and measured in the saliva-STAT instrument (N=3). Error bars represent +/− one standard deviation.

FIG. 56 show compiled saliva STAT test results using 500 μL saliva samples with varying concentrations of SARS-COV-2.

FIG. 57 shows it is advantageous to utilize larger sample volumes. The saliva STAT correctly identified all of the larger-volume samples, but for the 25 μL samples, it only correctly identified 4/5 samples with 2 copies/μL and 2/5 samples with 0.2 copies/μL.

FIG. 58 shows cycle threshold values for RT-qPCR analysis of saliva samples spiked with SARS-COV-2.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

As used herein, unless otherwise indicated, the term “aliphatic” refers to branched or unbranched hydrocarbon groups that, optionally, contain one or more degree(s) of unsaturation. Degrees of unsaturation can arise from, but are not limited to, cyclic aliphatic groups. For example, the aliphatic groups/moieties are a C₁₆ to C₄₀ aliphatic group, including all integer numbers of carbons and ranges of numbers of carbons therebetween (e.g., C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). Aliphatic groups include, but are not limited to, alkyl groups, alkene groups, and alkyne groups. The aliphatic group can be unsubstituted or substituted with one or more substituent(s). Examples of substituents include, but are not limited to, various substituents such as, for example, halogens (—F, —Cl, —Br, and —I), azide group, aliphatic groups (e.g., alkyl groups, alkene groups, alkyne groups, and the like), aryl groups, hydroxyl groups, alkoxide groups, carboxylate groups, carboxylic acid groups, ether groups, ester groups, amide groups, thioether groups, thioester groups, and the like, and combinations thereof.

As used herein, unless otherwise indicated, the term “alkyl group” refers to branched or unbranched saturated hydrocarbon groups. The alkyl group can be a C₁₆ to C₄₀ alkyl group, including all integer numbers of carbons and ranges of numbers of carbons there between (e.g., C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The alkyl group can be unsubstituted or substituted with one or more substituents. Examples of substituents include, but are not limited to, various substituents such as, for example, halogens (e.g., —F, —Cl, —Br, and —I), aliphatic groups (e.g., alkyl groups, alkenyl groups, and alkynyl groups), aryl groups, alkoxide groups, carboxylate groups, carboxylic acids, ether groups, alcohol groups, amine groups, thiol groups, thioether groups, and the like, and combinations thereof.

This disclosure provides a method for detecting the presence of an analyte in a sample by the use of thermally responsive aliphatic partitions (TRAPs) and magnetic beads. The thermally responsive aliphatic partitions may operate in at least two distinct behavior modes: (1) as a removable partition for hands-free reagent mixing following melting, and (2) as a continual partition that separates assay regions while enabling magnetic beads to be pulled through following melting, to enable hands-free immune-magnetic assays. Also provided are devices that utilize a method of the present disclosure.

In an aspect, the present disclosure provides a method of detecting an analyte of a sample. In some examples, the analyte may be detected to determine the disease state of an individual (e.g., whether or not the individual has a viral infection or whether or not the individual possesses antibodies for a particular viral infection).

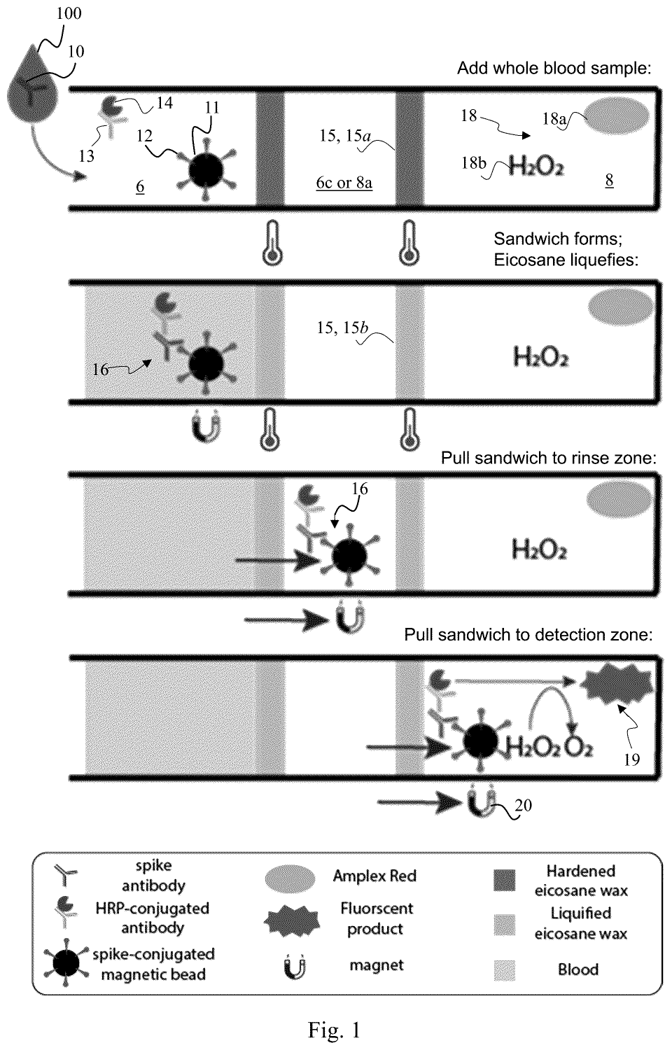

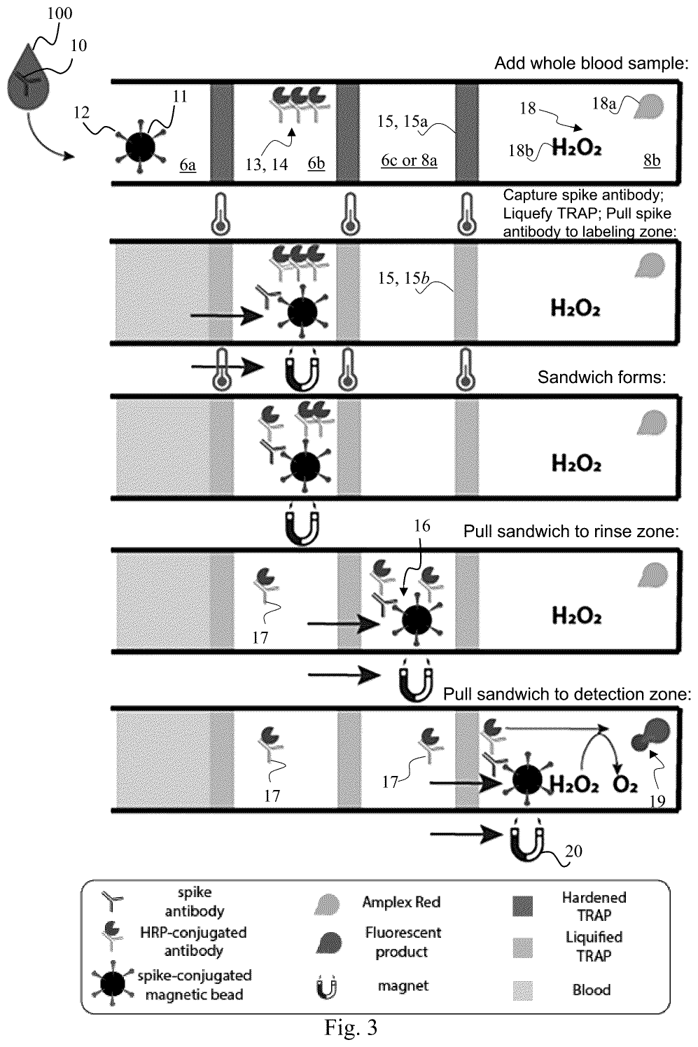

In various examples of a method for detecting the presence of an analyte in a raw sample may comprise using a thermally responsive aliphatic partition that separates adjacent assay regions while enabling magnetic beads to be moved into adjacent regions following melting of the aliphatic partition (FIG. 1). “Raw sample” may be used interchangeably with the term “sample.” “Melted partition” may be used interchangeably with the term “liquefied partition.” This example may comprise the following steps:
contacting a raw sample, such as a blood sample 100, that comprises or is suspected of comprising the analyte of interest 10, with (1) a plurality of magnetic beads 11 that have a plurality of first capture molecules 12 attached thereto, where the first capture molecules 12 have a specific affinity for the analyte 10 within the sample (e.g., blood sample 100), and (2) a plurality of second capture molecules 13 having a specific affinity for the analyte 10 in order to form complexes 16. The second capture molecules have a detectable label 14 attached thereto. The complexes 16 comprise the analyte 10 bound to the first capture molecule 12 attached to the magnetic bead 11 and the second capture molecule 13 bound to the analyte 10;
heating the aliphatic partitions 15 to a temperature at or between 40° C. to 65° C. such that aliphatic partitions 15 liquefy. At temperatures below 40° C., the aliphatic partitions are in a solid state 15a and limit (e.g., restrict or prevent) the movement of the complexes 16, and at temperatures at or between 40° C. to 65° C., the aliphatic partitions are in a liquid state 15b;
separating the complexes 16 from unbound second capture molecules 17 (shown in FIG. 3) by selectively moving the complexes 16 through one or more liquefied aliphatic partitions 15b;
detecting and optionally quantifying the signal generated from the detectable labels 14 of the second capture molecules 13 of the separated complexes 16.

In various examples, the method comprises a heating step, wherein the one or more solid aliphatic partitions 15a are liquefied such that bound analyte may be moved through the liquefied aliphatic partitions 15b via application of a magnetic field.

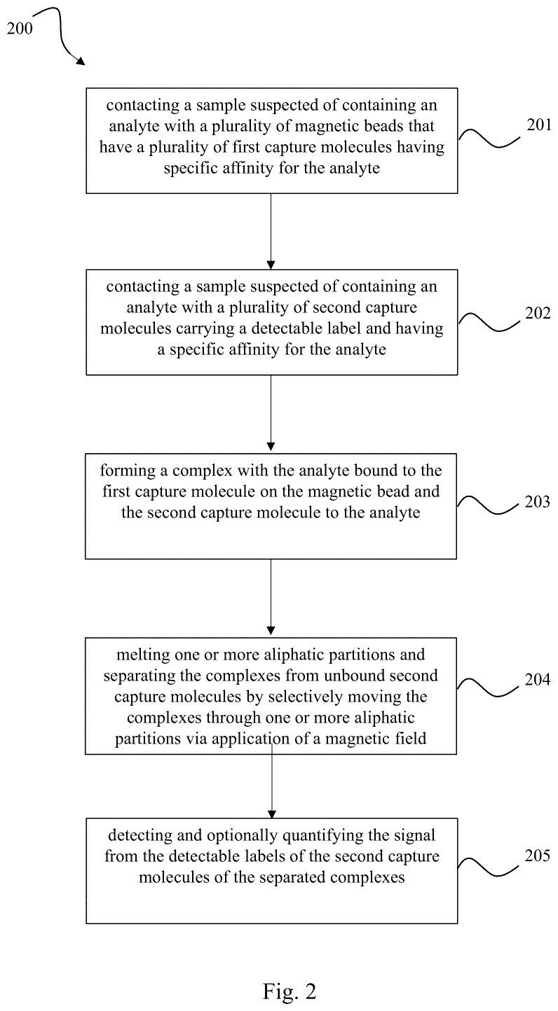

FIG. 2 displays a block diagram of the method disclosed herein. The method 200, may be broken into 5 distinct steps, 201, 202, 203, 204, and 205. Step 201 may comprise contacting a sample suspected of comprising an analyte with a plurality of magnetic beads that have a plurality of first capture molecules attached thereto, where the first capture molecules have a specific affinity for the analyte. Step 202 may comprise contacting a sample suspected of comprising an analyte with a plurality of second capture molecules having a detectable label attached thereto, where the second capture molecules have a specific affinity for the analyte. Step 203 may comprise forming a complex with the analyte bound to the first capture molecule and the second capture molecule to the analyte. Step 204 may comprise melting one or more aliphatic partitions separating adjacent regions and separating the complexes from unbound second capture molecules by selectively moving the complexes through one or more liquefied aliphatic partitions via application of a magnetic field. And step 205 may comprise detecting and optionally quantifying the signal from the detectable labels of the second capture molecules of the separated complexes.

The detectable label 14, on the second capture molecule 13, may be detected directly or indirectly. For example, as shown in FIGS. 1 and 3, the detectable label 14 may be indirectly detected via reaction with a detection molecule 18a or otherwise catalyze a reaction with a detection molecule 18a to generate a detectable signal. In other examples, the detectable label 14 is directly detectable via spectrophotometry. In various examples, the detectable label 14 reacts with a detection molecule 18a that may be colorimetric, luminescent, or fluorescent, to generate a signal 19 that is proportional (e.g., directly proportional) to the concentration of the analyte 10 within the sample 100. In various examples, the signal 19 may be compared to a control that does not comprise the detection molecule, detection label, or combination thereof.

As shown in FIGS. 1 and 3, various examples of the method comprise moving the sample through at least two regions, including a binding region 6 and a detecting region 8. The regions may further be divided into sub-regions. For example, the binding region 6 may be divided into one or more sub-regions. Examples of sub-regions include a first sub-region 6a comprising the first capture molecules 12 attached to magnetic beads 11, a second sub-region 6b comprising the second capture molecule attached to a detectable label, and one or more rinse regions 6c. For example, the binding region 6 may sequentially comprise the following sub-regions: a first binding sub-region 6a comprising the plurality of magnetic beads 11 and the first capture molecules 12, a rinse sub-region 6c, a second binding sub-region 6b comprising the second capture molecules 13 and the detectable label 14, where each sub-region is separated by an aliphatic partition 15. In various examples, the binding region 6 further comprises a second rinse sub-region 6c adjacent to the second-binding sub-region 6b and the sub-regions are separated by an aliphatic partition 15. In various examples, the binding region 6 comprises a binding sub-region comprising the plurality of magnetic beads 11, the first capture molecules 12, the second capture molecules 13, and the detectable label 14, and rinsing sub-region 6c, where the sub-regions are separated by an aliphatic partition 15. The detecting region 8 may optionally comprise one or more sub-regions. For example, the detecting region 8 may comprise a rinse sub-region 8a and a detecting sub-region 8b, where the detection sub-region 8b and rinse sub-region 8a are separated by an aliphatic partition 15. An aliphatic partition 15 would separate each region/sub-region.

The one or more rinse regions/sub-regions comprise an aqueous medium. In various other examples, the rinse region/sub-region may comprise water. In various other examples, the one or more rinse regions/sub-regions may further comprise phosphate buffered water, phosphate buffered saline, or Tris-buffered water.

In various examples, the binding regions/sub-regions and/or detecting regions/sub-regions comprise an aqueous medium. The binding regions/sub-regions and/or detecting regions/sub-regions comprise water. In various other examples, the binding regions/sub-regions and/or detecting regions/sub-regions may further comprise phosphate buffered water, phosphate buffered saline, or Tris-buffered water.

As shown in FIG. 3, the binding region/sub-region may comprise the plurality of magnetic beads 11 that have a plurality of first capture molecules 12 attached thereto, where the first capture molecules have a specific affinity for the analyte 10. Further, the binding region/sub-region may comprise a plurality of second capture molecules 13 having a detectable label 14 attached thereto, where the second capture molecules have a specific affinity for the analyte 10. The first capture molecules 12 and second capture molecules 13 may bind to different sections (e.g., epitopes) of the analyte 10 of the sample 100. Rinse sub-regions may be utilized to wash the sample.

As shown in FIGS. 1 and 3, in various examples, the detecting region 8 may comprise a molecule to initiate detection. For example, the chemical reaction that yields a product that may be detected via spectrophotometry. For example, if the second capture molecule 13 comprises horseradish peroxidase (HRP) as the detectable label 14, the detecting region 8 may comprise a detection composition 18, which comprises one or more detection molecules (e.g., Amplex Red, Luminol, or 3,3′,5,5′-Tetramethylbenzidine (TMB) 18a) and hydrogen peroxide 18b. In various examples, there is an interaction (e.g., chemical reaction) with the detectable label 14 on the second capture molecule 13 and the constituents of the detection composition 18. This interaction may be a chemical reaction. The chemical reaction may yield a fluorescent product 19. For example, when Amplex Red is utilized, the detectable label 14 may be HRP. In the presence of hydrogen peroxide 18b and Amplex Red 18a, HRP catalyzes the formation of a molecule that may be detected via fluorescence spectroscopy. It may be appreciated that the detection molecule 18a is not limited to Amplex Red, and may comprise other detection molecules, such as Luminol and 3,3′,5,5′-Tetramethylbenzidine (TMB).

As shown in FIGS. 1 and 3, a magnetic field is used to move the complexes 16 through the various regions utilized in the method. The magnetic field may be generated by a magnet (e.g., an external magnet), such as, for example, a neodymium magnet. In various other examples, the magnetic field is generated by a wire through which a current is applied. In various examples, an external magnet 20 is moved at a rate of 0.2 mm/s to 10 mm/s in the direction from the binding region 6 to the detecting region 8. In various other examples, the magnet is stationary and is positioned such that an application of a magnetic field in the absence of moving the magnet induces movement of the complexes 16 through the various regions utilized in the method. In various other examples, the magnet is stationary and a testing assembly in which the method is occurring is moved relative to the magnet. In various examples, the cartridge may move, the magnet may move, or both may move relative to each other to effect the movement of the magnetic beads along the sequential path. Without intended to be bound by any particular theory, it is considered that a magnet or device capable of generating a magnetic field with a pull force of 5-40 pounds and/or a strength of 10-2 to 10 tesla.

As shown in FIG. 3, in various examples, each region described in the method is separated by an aliphatic partition 15. Sample 100 comprising the analyte 10 is contacted with the components in the binding region 6, and the analyte 10 binds to the first capture molecule 12. The solid aliphatic partitions 15a are heated to a temperature from 40° C. to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.), to melt the aliphatic partitions into liquefied partitions 15b. The analyte 10 bound to first capture molecules 12 attached to the magnetic beads 11, which are moved through the permeable liquefied aliphatic partitions 15b via application of a magnetic field. The analyte may then optionally pass through a rinse sub-region 6c or 8a following moving through a first liquefied aliphatic partition 15b. The analyte 10 is isolated from the sample 100 (e.g., whole blood) into a separate second sub-region 6b comprising the plurality of second capture molecules 13 having a detectable label 14 attached thereto. The second capture molecules 13 bind to the captured analyte 10 to form complexes 16 that comprise the analyte 10 bound to the first capture molecule 12 on the magnetic bead 11 and the second capture molecule 13 having the detectable label 14 attached thereto. The detectable label 14 remains bound to the second capture molecule 13 in this complex 16. Complex 16 may then optionally be moved through a rinsing sub-region 6c or 8a. Finally, the complexes 16 are moved into the detecting region comprising a detection composition 18, which comprises hydrogen peroxide 18b and Amplex Red 18a. The interaction (e.g., chemical reaction) between the detectable label 14, which is HRP and the detection composition 18 yields a fluorescent product 19 that may be detected by a fluorescent signal that is proportional (e.g., directly proportional) to the concentration of the analyte 10.

As shown in FIG. 1, in various examples, sample 100 comprising the analyte 10 is contacted with the components in the binding region 6, and the analyte 10 binds to the first capture molecule 12 attached to the magnetic bead 11 and the plurality of second capture molecules 13 having a detectable label 14 attached thereto. The second capture molecules 13 bind to the captured analyte 10 to form complexes 16 comprising of the analyte 10 bound to the first capture molecule 12 attached to the magnetic bead 11 and the second capture molecule 13. The solid aliphatic partitions 15a are heated to a temperature from 40° C. to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.), to melt the aliphatic partitions 15 into liquefied aliphatic partitions 15b. The magnetic beads 11 bound to the analyte 10 are moved through the liquefied aliphatic partitions 15b via application of a magnetic field. The complex 16 is optionally moved through a rinse sub-region 6c or 8a and through a liquefied aliphatic partition 15b. Complex 16 is moved into the detecting region 8. The detecting region 8 may comprise a detection composition 18 that comprises hydrogen peroxide 18b and Amplex Red 18a. The chemical reaction between the detectable label 14, which is HRP and the detection composition 18 yields a fluorescent product 19 that may be detected by a fluorescent signal that is proportional to the concentration of the analyte 10.

In an example, various analytes may be detected using a method of the present disclosure. For example, the analyte may be an antigen, an antibody, a viral particle, or the like. For example, the antigen may be a pathogenic antigen (e.g., microbial antigen, bacterial antigen, or viral antigen). Non-limiting examples of viruses from which the antigen, antibody, or viral particle are associated include SARS-COV-2, Hepatitis C, Epstein-Barr, Zika, Ebola, Herpes simplex, Norovirus, Influenza, Chikungunya, or the like. In various examples, the analyte is associated with SARS-COV-2. For example, the analyte is a SARS-COV-2 antibody.

The method may utilize various capture molecules. For example, the capture molecules comprise antibodies having a specific affinity for the analyte. The antibodies of the first capture molecule 12 and the second capture molecule 13 may be different and bind to different epitopes of the analyte 10.

The first capture molecule 12 has a magnetic bead 11 attached thereto. In various examples, the first capture molecule is streptavidin. The magnetic bead 11 may comprise iron oxide and be superparamagnetic. The magnetic beads 11 may have a longest linear dimension of 100 nm to 50,000 nm, including all 0.1 nm values and ranges therebetween. In various examples, the mean diameter of the magnetic beads 11 is about 1 μm. In various examples, the magnetic beads have a density of about 2 g/cm³. In various aspects, the magnetic beads 11 are coated with streptavidin.

The second capture molecule 13 has a detectable label 14 attached thereto. The second capture molecule may be The detectable label 14 may be detected directly or indirectly. For example, a detectable label 14 that is indirectly detected may catalyze or undergo a chemical reaction in the detecting region 8 with a substrate, resulting in the formation of a detectable product (e.g., detection molecule) that may be detected and optionally quantified via spectrophotometry. For example, a detectable label 14 is directly detected and optionally quantified via spectrophotometry without formation of an additional substrate or detection molecule 18a. For example, a direct detectable label is green fluorescent protein (GFP).

According to an example of the present disclosure, the sample 100 may be a biological sample. Non-limiting examples of samples include whole blood, blood fractions, plasma, serum, saliva, urine, stool, sweat, mucous, tears, breast milk, semen, tissue, placental tissue, bone marrow, conditioned medium, tissue culture medium, and the like.

Various aliphatic partitions 15 may be used. For example, the aliphatic partitions 15 may comprise one or more alkanes. The alkanes may have a melting point of 40 to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.). At the melting temperature, the aliphatic partition 15 undergoes a phase change from a solid aliphatic partition 15a to a liquefied aliphatic partition 15b allowing the movement of the complexes 16 through the partition 15b. Non-limiting examples of alkanes include eicosane, docosane, hexacosane, heptacosane, nonococane, tetracosane, octadecane, and combinations thereof. In various other examples, the aliphatic partition 15 may be any fatty acid with a melting point of 40 to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.). In a method of the present disclosure, the aliphatic partitions 15 are initially solid 15a to prevent movement of media adjacent regions and then subsequently melted 15b to allow for movement of the analyte from region to adjacent region via application of a magnetic field.

In various examples, the detection composition 18 may comprise a detection molecule 18a that may be colorimetric, luminescent, or fluorescent or result in a detected molecule that may be colorimetric, luminescent, or fluorescent. In various examples, the detection molecule 18a may be indirectly or directly detected and optionally quantified after a chemical reaction. The detection molecule 18a or detected molecule may be detected visually or via spectrophotometry. In various examples, the detection molecule 18a and the detectable label 14 and detected molecule are the same (e.g., green fluorescent protein). In an example, a colorimetric detection molecule 18a may be 3,3′,5,5′-Tetramethylbenzidine (TMB) or the like. In an example, a luminescent detection molecule 18a may be Luminol or the like. In an example, a fluorescent detection molecule 18a may be Amplex Red or the like. Other examples of detection molecules include, but are not limited to 2,2′-azino-bis(3-ethylbenzothiazoline-6-sulfonic acid (ABTS), 3-amino-9-ethylcarbazole (AEC), 3,3′-diaminobenzidine (DAB), and the like.

In various examples, the detection molecule 18a may comprise 3,3′,5,5′-Tetramethylbenzidine (TMB) and the secondary capture molecule 13 may have horseradish peroxidase (HRP) tag attached thereto as the detectable label 14. In such an example, the detection solution 18 may further comprise hydrogen peroxide 18b. In the presence of hydrogen peroxide and TMB, HRP catalyzes the formation of a molecule that may produce a color change. The color change may be measured, assessed, or evaluated by an LED, an optical filter, or an optical detector, such as a photodetector or a camera.

In an example, the detection molecule 18a may comprise Luminol and the secondary capture molecule 13 may have a horseradish peroxidase (HRP) tag attached thereto as the detectable label 14. In the presence of hydrogen peroxide and Luminol, HRP catalyzes the formation of a molecule that may produce luminescent product. The luminescent product may be measured, assessed, or evaluated by an optical detector, such as a photodetector or a camera. The term detection molecule may refer to the substrate that is reacted to form the detected molecule.

In an example, the detection molecule 18a may comprise Amplex Red and the secondary capture molecule 13 may have a horseradish peroxidase (HRP) tag attached thereto as the detectable label 14. In the presence of hydrogen peroxide and Amplex Red, HRP catalyzes the formation of a molecule that may produce a fluorescent product 19. The fluorescent product 19 may be measured, assessed, or evaluated by an LED, an optical filter, or an optical detector, such as a photodetector or a camera.

In various examples, the detectable label 14 may be a fluorescent molecule that is tagged on the second capture molecule 13. The fluorescent molecule may be a fluorophore such as, but not limited to, derivatives of fluorescein, derivatives of rhodamine (TRITC), coumarin, GFP, or cyanine. The FITC-conjugated second capture molecule may produce a fluorescent product that may be measured, assessed, or evaluated by an LED, an optical filter, or an optical detector, such as a photodetector or a camera.

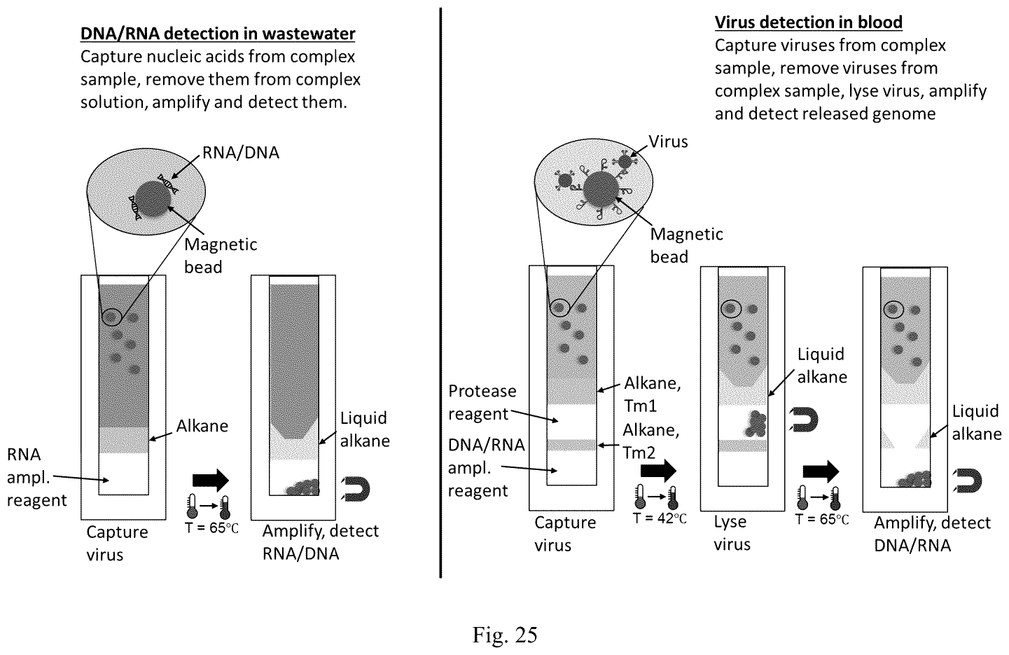

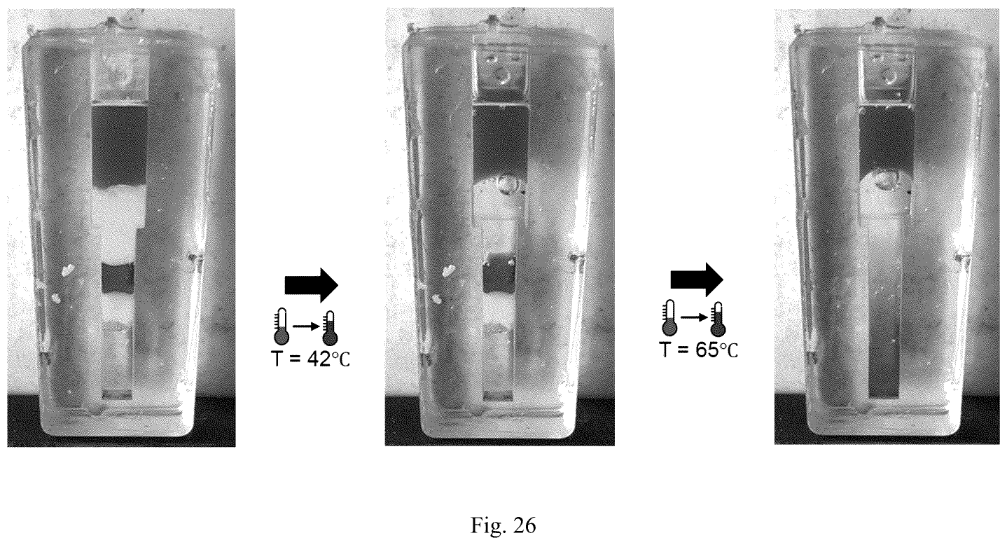

In an aspect, the present disclosure provides a method for detecting a blood borne virus. The method may be adapted to be used in a system of the present disclosure. Various blood borne viruses may be detected via this method. A schematic/cartoon depicting this method is found in FIG. 25 and Example 5.

In various examples, the method occurs in a system (e.g., a cassette) comprising a plurality of linearly adjacent chambers, where each chamber separated by an aliphatic partition. For example, the system comprises a first chamber linearly adjacent to a first aliphatic partition that has a melting point of or around 40° C. The system may further comprise a second chamber linearly adjacent to the first aliphatic partition. The second chamber is also linearly adjacent to a second aliphatic partition having a melting point above 50° C. and has a longest linear dimension that is less than the first aliphatic partition. That is, the first aliphatic partition is thicker than the second aliphatic partition and the second chamber is between the two aliphatic partitions. An example of this system presented in FIG. 25. The system may further comprise a third chamber that is linearly adjacent to the second aliphatic partition. The first chamber may comprise a plurality of magnetic beads having capture molecules conjugated thereto. The second chamber may comprise one or more proteases. The third chamber may comprise reagents necessary for genomic amplification.

The method may comprise the following steps: contacting in a first chamber a sample (e.g., blood) suspected of containing a viral analyte with a plurality of magnetic beads having a plurality of capture molecules (e.g., aptamers) attached thereto, where the capture molecules have an affinity for the viral analyte. In various examples, the capture molecules bind and have an affinity for an antigen on the surface of the viral analyte. Upon contact with the analyte and the capture molecules, a complex comprising the analyte and capture molecule attached the magnetic bead is formed. Following complexation, the entire complex and an adjacent solid aliphatic partition is heated to a temperature of 35 to 45° C. such that the solid aliphatic partition liquefies. In various examples, the aliphatic partition is eicosane. After the partition is liquefied, the complex is moved from the first chamber into a second chamber via application of a magnetic field. The second chamber may comprise a protease that is capable of lysing the viral antigen such that its viral genome is released. Following lysing, the system is heated to a temperature greater than 50° C. This heating step may deactivate the protease and liquefy the second aliphatic partition and induce a breach such that the contents of the second chamber and third chamber mix. Following mixing of the second and third chamber, the viral genome is then amplified and detected.

Various methods may be used to detect the amplified material. In various examples, the formation of the new DNA may be detected via the use of an intercalating dye. As fluorescence of this dye increases, it is expected that as does the amount of DNA formed.

In various examples, DNA or RNA in a sample may be detected. Such a method may utilize a system having a plurality of linearly aligned chambers, each chamber separated by an aliphatic partition. For example, the system comprises a first chamber comprising magnetic beads having capture molecules conjugated thereof and a second chamber comprising the reagents necessary for DNA and/or RNA amplification. The two chambers may be separated by an aliphatic partition. A method using such a system may comprise contacting in the first chamber the sample suspected of containing DNA or RNA with magnetic beads having capture molecules conjugated thereto. Upon the DNA or RNA contacting the capture molecule to which it has a binding affinity, a complex is formed. In various examples, the capture molecules are aptamers having an affinity for a specific DNA or RNA. The system may then be heated such that the aliphatic partition liquefies. The complex may then be transported from the first chamber through the aliphatic partition to the second chamber via application of a magnetic field. When the captured DNA and/or RNA is in the second chamber, it may then be amplified and detected. Various methods may be used to detect the amplified material. In various examples, the formation of the new DNA may be detected via the use of an intercalating dye or other detectable molecule. As fluorescence of this dye increases, it is expected that as does the amount of DNA formed.

In an aspect, the present disclosure provides a system to perform genomic detection of viruses in blood samples. The system comprises a cassette and a handheld instrument comprising a heating element, a magnetic control, an LED, and a camera or optical detector.

A cassette may comprise a plurality of linearly adjacent chambers, where each chamber separated by an aliphatic partition. For example, the system comprises a first chamber linearly adjacent to a first aliphatic partition that has a melting point of or around 40° C. The system may further comprise a second chamber linearly adjacent to the first aliphatic partition. The second chamber is also linearly adjacent to a second aliphatic partition having a melting point above 50° C. and has a longest linear dimension that is less than the first aliphatic partition. That is, the first aliphatic partition is thicker than the second aliphatic partition and the second chamber is between the two aliphatic partitions. An example of this system presented in FIG. 25. The system may further comprise a third chamber that is linearly adjacent to the second aliphatic partition. The first chamber may comprise a plurality of magnetic beads having capture molecules conjugated thereto. The second chamber may comprise one or more proteases. The third chamber may comprise reagents necessary for genomic amplification.

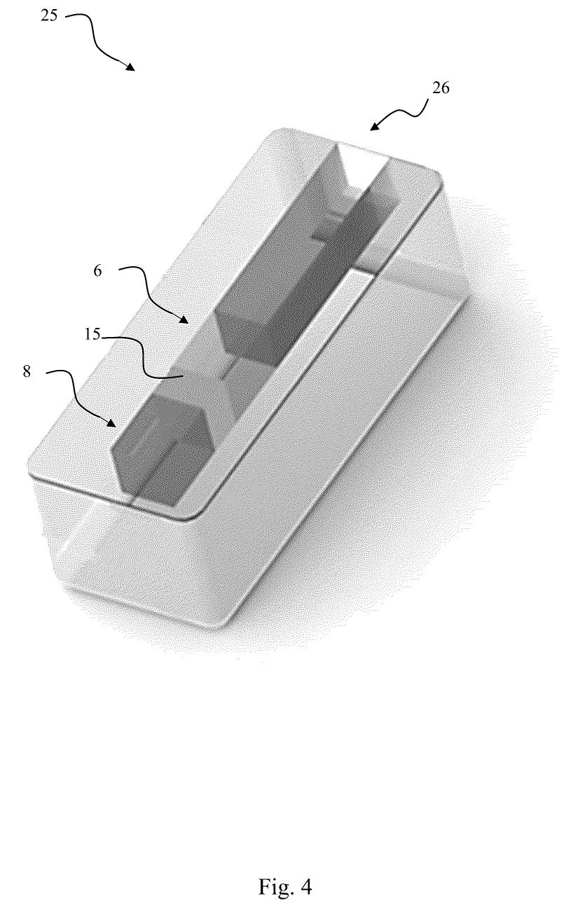

In an aspect, the present disclosure provides a testing system for detecting the presence of an analyte using the combination of thermally responsive aliphatic partitions (TRAPs) and magnetic beads (FIG. 4).

Referring to FIGS. 1, 3, and 4, the testing system may comprise a test assembly 25 and a magnet 20 or source to generate a magnetic field. FIG. 4 displays the test assembly 25. The test assembly 25 comprises an inlet 26 configured to accept a sample 100, a plurality of regions that include a binding region 6 and a detecting region 8, and at least one aliphatic partition 15 disposed therein. Adjacent regions of the plurality of regions are separated by at least one aliphatic partition 15. The binding region 6 comprises a plurality of magnetic beads 11 connected to a plurality of first capture molecules 12 and a plurality of second capture molecules 13 connected to a detectable label 14. The plurality of first capture molecules 12 are configured to bind to an analyte 10 present in a sample 100. The plurality of second capture molecules 13 are configured to bind to the analyte 10 present in the sample 100. The detecting region 8 comprises a detection composition 18 that allows spectrophotometric measurement of the analyte directly or indirectly. The magnet 20 is configured to apply a magnetic field along a sequential path of the test assembly 25. The sequential path includes the binding region 6 and the detecting region 8. The plurality of magnetic beads 11 move sequentially along the sequential path upon application of a magnetic force from the magnet 20. Thus, the magnetic beads 11 can move sequentially from the binding region 6 to the detecting region 8. The magnetic beads 11 also can move sequentially through other regions or sub-regions (such as first sub-region 6a, second sub-region 6b, binging region rinse region 6c, detecting region rinse region 8a, or detecting region 8b) along the sequential path, but in an example the magnetic beads 11 move from one side of the sequential path to the other side of the sequential path.

The channel may have various shapes. For example, the channel may be cylindrical, prism-shaped, rectangular cuboid, or the like. The aforementioned shapes are merely illustrative. Other shapes are contemplated within the scope of the instant disclosure. While a uniform channel width is disclosed, other configurations using different widths, a tapering width, or a widening width are possible.

In various examples, a testing assembly with a 3×3 mm channel may be used. This width allows the magnetic beads 11 to pass through the aliphatic partitions 15 without breaching (meaning mixing the contents within the binding region 6 with the contents within the detecting region 8), and the layers of the assay remain separated during the melting of the aliphatic partitions 15, moving the magnetic beads 11 across liquefied aliphatic partitions 15b, and re-hardening of the aliphatic partitions 15 into solid aliphatic partitions 15a. Solid aliphatic partitions 15a melt into liquefied aliphatic partitions 15b by heating the aliphatic partitions 15 to a temperature at or between 40 to 65° C. (e.g., 40, 41, 42, 43, 44, 45, 46, 47, 48, 49, 50, 51, 52, 53, 54, 55, 56, 57, 58, 59, 60, 61, 62, 63, 64, or 65° C.). The regions and sub-regions are positioned in this channel.

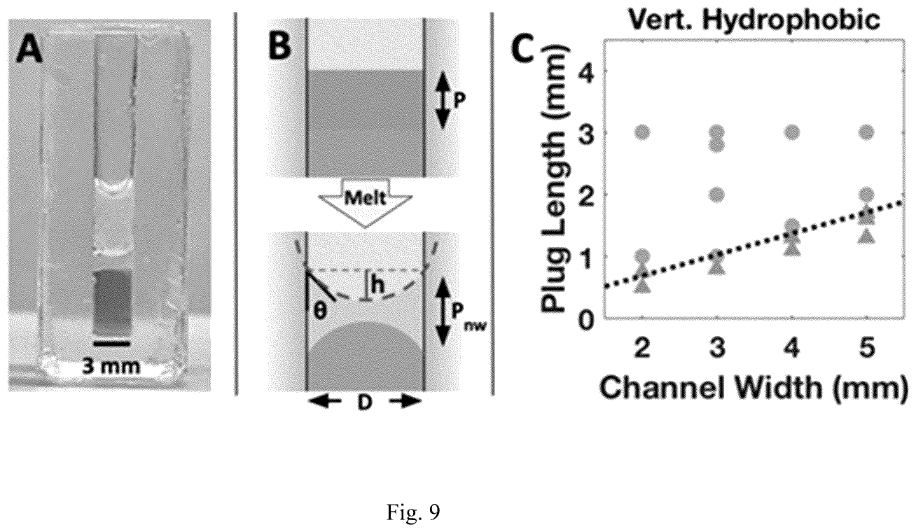

According to an example of the present disclosure, at least one aliphatic partition 15, the binding region 6, and the detecting region 8 may be arranged in a horizontal channel or a vertical channel. Further, the horizontal channel or the vertical channel may be hydrophobic or hydrophilic, as shown in FIGS. 9-12, and discussed infra Example 1.

According to various examples of the present disclosure, the aliphatic partitions 15 may comprise one or more alkanes chosen from eicosane, docosane, hexacosane, heptacosane, nonococane, tetracosane, and octadecane. Combinations of alkanes or additional species with the alkanes are possible. The aliphatic partitions 15 may operate in at least two distinct behavior modes: (1) as a removable partition for hands-free reagent mixing following melting, and (2) as a continual partition that separates assay regions while enabling magnetic beads 11 to be pulled through following melting. The mode that the aliphatic partition 15 operates in is dependent on the geometry, position, and thickness of the aliphatic partition 15. Because of the density and polarity differences between the alkane and the aqueous reagents, the behavior is dependent upon the surface energy of the test assembly 25 and the orientation of the test assembly 25. The aliphatic partitions 15 can remain between the various regions or sub-regions and can obstruct flow of material between the regions or sub-regions except for the magnetic beads 11. The aliphatic partitions 15 can extend across an entirety of the channel in the test assembly 25 to provide this function.

According to various examples of the present disclosure, one aliphatic partition 15 may be configured to separate the binding region 6 and the detecting region 8. Further, the binding region 6 may comprise a plurality of sub-regions, each adjacent region or sub-region separated by one of the aliphatic partitions 15. A first binding sub-region 6a of the binding region 6 may comprise the plurality of magnetic beads 11 connected to the plurality of first capture molecules 12 having a specific affinity to the analyte 10 present in the sample 100. A second binding sub-region 6b of the binding region 6 may comprise the plurality of second capture molecules 13 connected to a detectable label 14 having a specific affinity to the analyte 10 present in the sample 100. Even further, the testing assembly 25 may comprise a rinsing sub-region 6c or 8a disposed between the aliphatic partition 15 of the first binding sub-region 6a and the aliphatic partition 15 of the second binding sub-region 6b.

According to an example of the present disclosure, the magnet 20 is external to the test assembly 25, and can be moved at a rate of 0.2 mm/s to 10 mm/s along the sequential path that may comprise the binding region 6 and the detecting region 8. Thus, the magnet 20 can move along the test assembly 25 as shown in FIG. 1, 3, or 6. The magnet 20 may also be disposed on an end of the test assembly 25 in order for the magnetic field to extend across the binding region 6 and the detecting region 8 sequentially. In this configuration, the magnet 20 may be fixed or may stay localized at the end of the test assembly 25 (e.g., the short end of the testing assembly perpendicular to the movement illustrated in FIGS. 1 and 3).

According to an example of the present disclosure, the detecting region 8 may comprise one or more optically transparent faces in order to allow spectrophotometric measurement of the detection composition 18 within the detecting region 8.

According to an example of the present disclosure, the detection composition 18 may comprise a detection molecule 18a that may be colorimetric, luminescent, or fluorescent, and hydrogen peroxide 18b. In various examples, a colorimetric detection molecule may be 3,3′,5,5′-Tetramethylbenzidine (TMB). In various examples, a luminescent detection molecule may be Luminol. In various examples, a fluorescent detection molecule may be Amplex Red.

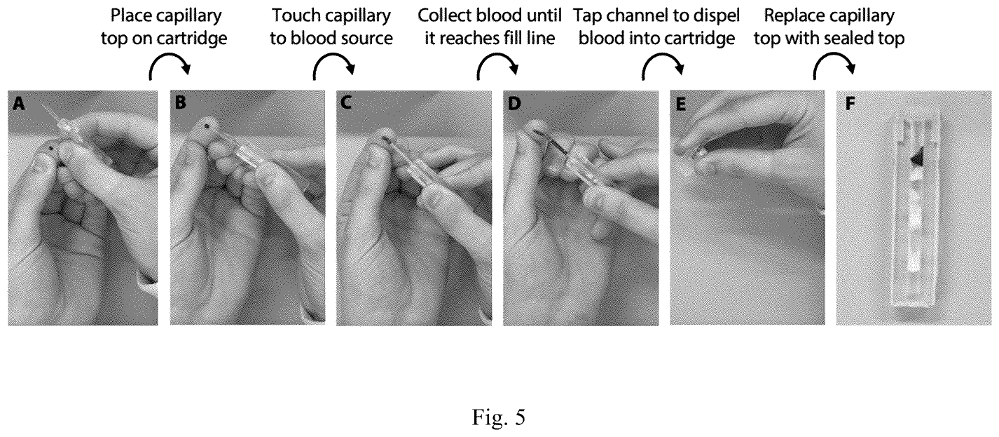

FIG. 5 demonstrates the capture of the sample 100 to be inserted into the inlet 26 of the test assembly 25. As shown in FIG. 5A, a capillary tube may prick a finger of a subject. When the capillary tube is contacted with the blood sample (FIG. 5B), the tube wicks the blood (FIG. 5C) until a precise volume is reached (FIG. 5D). The capillary tube may be inserted into the inlet 26 of the test assembly 25. The capillary tube may be tapped in order to enter the blood into the test assembly 25 (FIG. 5E). The capillary tube is exchanged with a sealed cap after the sample is loaded (FIG. 5F).

In various examples, one or more of the surfaces of the channel are rendered hydrophilic. They may be rendered by contacting the one or more surfaces with fetal bovine serum (FBS). Without intending to be bound by any particular theory, it is considered that the contacting increases the hydrophilicity of one or more surfaces.

This disclosure further provides a method of using the test assembly 25 for detecting the presence of an analyte 10. The capillary tube may transport the sample 100 needed into the test assembly 25 in order to perform the method 200 described herein.

### Portable Histone Assay Technology (PHAST) Platform Embodiment

Histones, recognized as damage-associated molecular patterns (DAMPs), are linked to cell death. Elevated histone levels in the blood have previously been associated with multiple organ failure and/or damage caused by circulating histones. For this reason, the presently disclosed device (the “PHAST” device) detects histone levels in the blood. An assay uses double-stranded DNA (dsDNA) and the intercalating dye, EvaGreen, to which the PHAST device measures the corresponding fluorescence. Histones are well known for their high affinity for DNA and preferentially bind with DNA when in a solution. EvaGreen, moreover, has an affinity for dsDNA, albeit to a lesser extent compared to the histone-DNA binding. Based on these interactions, the presence of histones causes dsDNA to wrap around them, preventing EvaGreen from binding to the DNA and fluorescing. Conversely, in the absence of histones, the dsDNA is free to interact with Evagreen, allowing for fluorescence. This establishes an inverse relationship between the fluorescence intensity of the solution and the histone concentration.

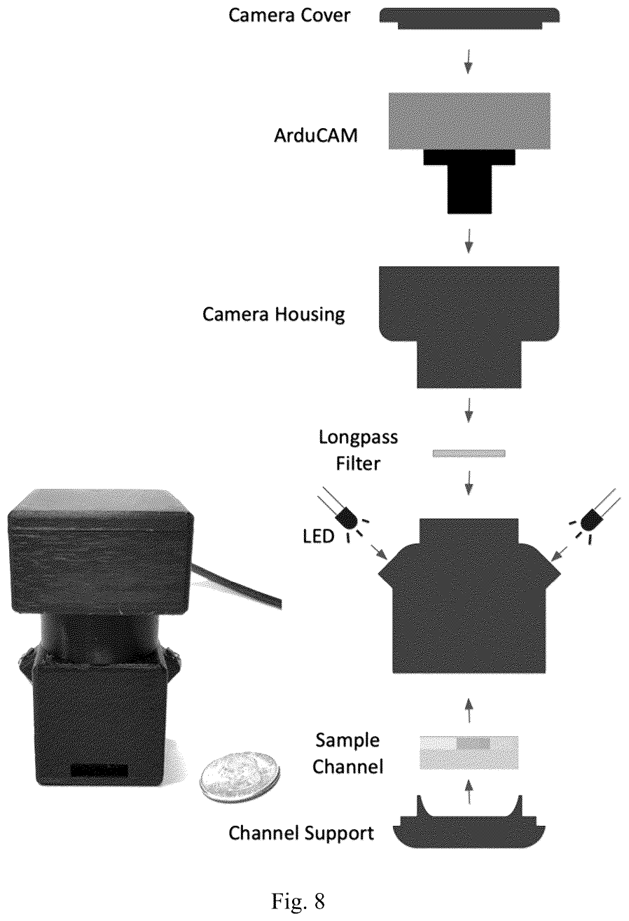

To ensure user-friendly point-of-care functionality, the assay is designed as a sample-to-answer test. Blood is added directly into a tube and the tube is then inserted into the device to be measured with no additional steps for the user. For this purpose, all reagents, excluding the patient's blood, are present so that the user needs only to add the blood sample for the test. However, the DNA solution is kept separate from the EvaGreen to prevent binding. This is accomplished through thermally responsive alkane partitions (TRAPs) which separate the DNA solution (above a wax partition) from the EvaGreen-Dextran solution (below the wax partition). Blood is added directly on top of the wax where a 10-minute incubation step occurs to allow for the DNA to wrap around the histone. Subsequently, the wax is melted at 45° C., allowing for the two layers to mix. Any unbound DNA binds to the EvaGreen and begins to fluoresce. Red blood cells (RBCs) are aggregated due to their interaction with dextran, originally below the wax with the EvaGreen solution. The aggregation and sinking of the RBCs allow for a clearer imaging window for measuring fluorescence, as blood has a high optical absorbance. The steps of the assay are shown in FIG. 8.

With reference to FIG. 9, the PHAST platform that measures the cartridges detailed above may include: (1) a processor, (2) an optical subassembly and heating chamber, (3) a battery and charge controller, and (4) a PC GUI.

The processor may be configured to: (1) toggle the excitation LED for fluorescence readings, (2) control an ultra-stable 24-bit ADC for measuring fluorescence emitting from the cartridge, (3) run a closed loop heating controller to adjust the temperature of the cartridge based on the previously described profile by monitoring a digital temperature sensor embedded in the main chamber and then sending power to a thermoelectric heating element, also on the chamber, and (4) send data to and receive data from a user interface (e.g., a PC-based GUI). In a prototype embodiment, the processor was a custom-designed Atmel328-based controller.

The optical subassembly includes a 465 nm excitation light source, such as, for example, an LED, mounted in a bracket adjacent to a heating chamber where the cartridge under test is located. This LED may be controlled by a MOSFET that is triggered on or off by the processor. Controlling LED actuation may beneficially reduce any photobleaching effects caused by persistent exposure to light.

The light from the LED passes through an aperture in the heating chamber such that only a predetermined section of the cartridge is illuminated. In some embodiments of the PHAST system, the fluorescing layer is only a few mm thick, so the excitation and emission apertures may be correspondingly narrow, generally only 1 mm in height. This provides that the device is primarily reading fluorescence, not changes in optical absorption in other areas caused by the wax or blood.

The fluorescence emission from the cartridge passes through another aperture, through a 534 nm OD6 (where OD is optical density) fluorescence bandpass filter, and to a photodiode. In another non-limiting embodiment, a bandpass filter is placed at the light source, and a filter at the photodiode is a 521 nm OD8. The resulting photocurrent from the photodiode processed and provided to the processor. For example, the photocurrent may be amplified by an ultra-high gain transimpedance amplifier whose voltage is then sampled by an analog-to-digital converted (ADC). Because the sensitivity for the PHAST device may be required to be high (+5 nM typ.), a special mode of the ADC may be used called single cycle settling in conjunction with a very slow low pass filter to provide an effective number of bits of 24 with u V noise performance. The ADC may sample a signal for almost three seconds before reporting a value, however this provides a very stable and sensitive measurement.

It may be advantageous to follow a temperature profile over the duration of a test. The heating chamber of the device may be fabricated from aluminum in order to allow rapid heat conduction and precise temperature control. A digital temperature sensor may be mounted to the heating chamber, which is then read by the processor. Depending on the current timepoint in the temperature profile, the processor may enable a heater to heat the chamber. For example, the processor may control a MOSFET to allow current to flow into a Peltier junction mounted on the heating chamber. While Peltier junctions are typically used as coolers, they can also be used to heat. For the present device, they have the advantage of being quite small, run at 5 volts, and can rapidly and efficiently heat a small volume. In a prototype embodiment, a Peltier junction was able to heat a cartridge from room temperature to 45° C. in under two minutes.

The PHAST system is intended as a fully portable device, so a large battery (such as a Lithium polymer (LiPo) battery) may be included, along with its associated charge controller. Because the heating requirements are fairly modest over one test, selecting a sufficiently sized battery will allow the device to run dozens of tests on one charge.

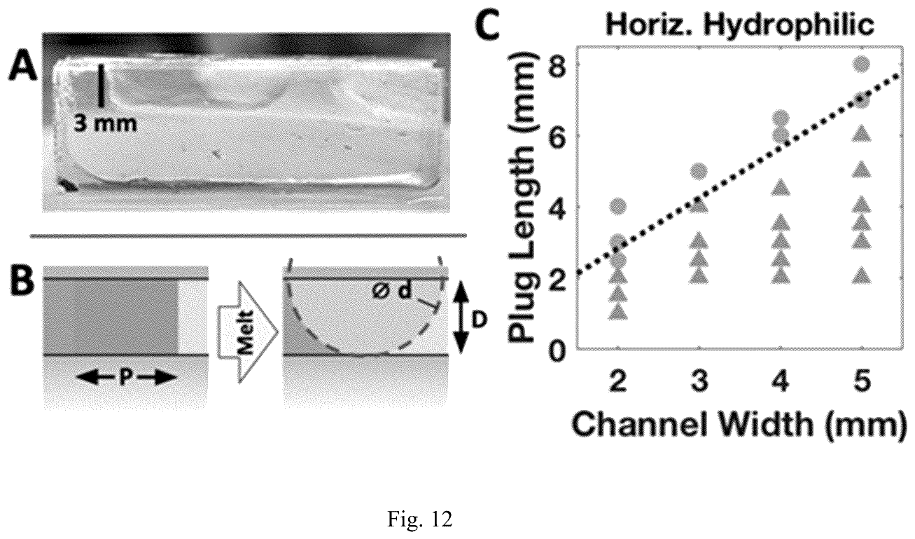

The PHAST system may include a graphical user interface (GUI) (see, for example, the GUI of the prototype embodiment shown in FIG. 12). For example, a Windows-based GUI may allow a user to start and record tests, change the heating profile parameters, and adjust a threshold value for what is considered a positive result. The device may have an indicator LED and start button. Once the various parameters are set using the GUI, they can be stored within the device (e.g., using a memory device in communication with the processor). In this way, a user has only to insert a sample cartridge, press the start button, and wait for the result to be displayed by the indicator (e.g., a color-coded indication (e.g., red, green) to provide a positive or negative result).

### Sample To Answer Test (STAT) Platform Embodiment

In another embodiment, a point of care device may further comprise a movable magnet (such as, for example, a movable permanent magnet).

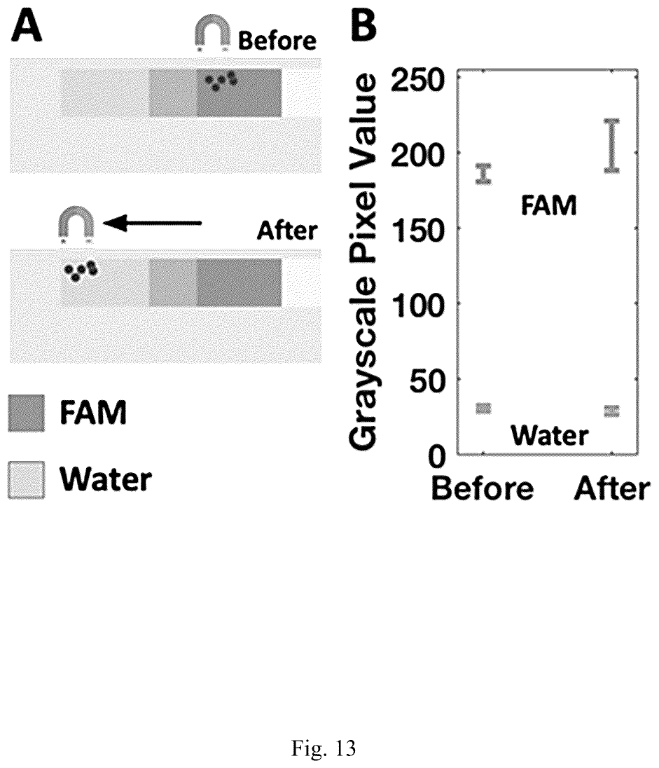

An example sample cartridge inserted into the diagnostic device (an example is pictured in FIG. 13) is configured as follows. At the bottom of a 2.5 mm diameter channel in the cartridge, sequestered below a hardened hexacosane partition, are reagents for an isothermal virus detection reaction. Between the hexacosane partition and a hardened eicosane partition are reagents for a virus lysis reaction. On top of the eicosane partition is a plurality of magnetic microbeads functionalized with virus capture molecules.

After a blood sample is added into the sample cartridge, the virus particles targeted by the virus capture molecules are immobilized onto the magnetic beads. A stored testing profile is used by the processor to cause the heater to increase the temperature of the cartridge to soften the eicosane. The magnetic microbeads are moved across the melted eicosane layer using the movable magnet, leaving behind the blood sample. Once the beads are across the softened eicosane partition (and thus located within the lysis reaction layer), the processor allows the temperature of the sample cartridge to decrease to allow lysis enzymes to break open virus particles to release their genome. After an incubation period, the processor again increases a temperature of the sample cartridge to inactivate the lysis enzymes, melt the hexacosane layer (resulting in the mixing of virus genome with the virus detection reaction), and initiate the detection reaction. FIG. 14 shows the full reaction temperature profile.

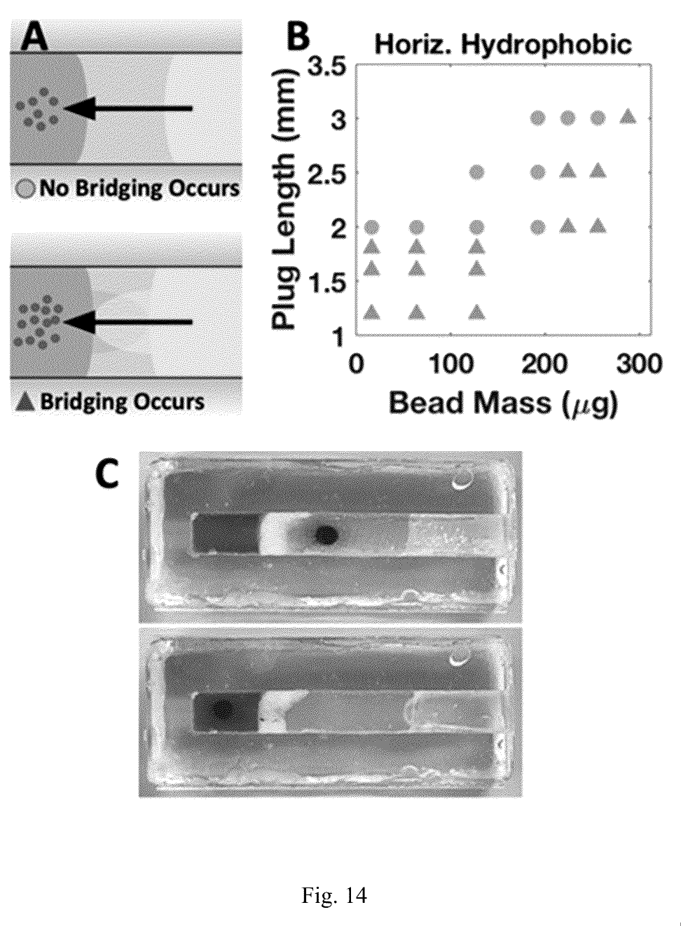

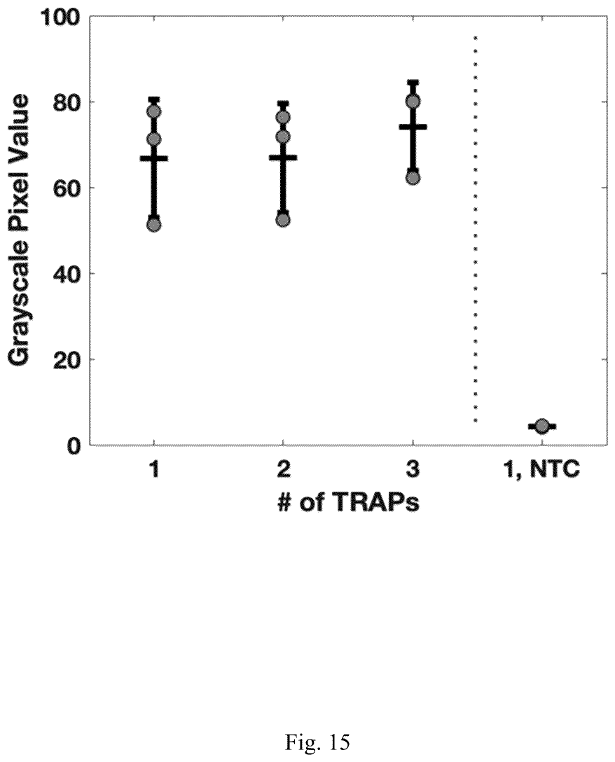

The STAT platform example that measures the cartridges detailed above includes five main components (FIG. 15): (1) a processor board, (2) an optical subassembly and heating chamber, (3) an electronically movable permanent magnet, (4) a battery and charge controller, and (5) a PC GUI.

The processor board is a custom-designed Atmel328 based controller that (1) toggles the excitation LED for fluorescence readings, (2) controls a ultra-stable 24 bit ADC for measuring the fluorescence emitting from the cartridge, (3) runs a closed loop heating controller to adjust the temperature of the cartridge based on the previously described profile by monitoring a digital temperature sensor embedded in the main chamber and then sending power to a thermoelectric heating element, also on the chamber, and (4) sends and receives data over a USB connection to a custom-made PC GUI.

The optical subassembly may include light source, such as, for example, a 465 nm excitation LED, mounted in a bracket adjacent to a heating chamber where the cartridge under test is located. This LED may be controlled by a MOSFET that is triggered on or off by the processor. Controlling LED actuation may beneficially reduce photobleaching effects caused by persistent exposure to light.

The light from the LED passes through an aperature in the heating chamber such that only a predetermined section of the cartridge is illuminated. This may be advantageous because, in some embodiments, extraneous light that impinges on the wax sections higher up or other parts of the device can create a much higher baseline signal that can saturate the detector and distort the real signal.

The fluorescence emission from the cartridge passes through another aperture, through a 520 nm OD6 fluorescence bandpass filter, and then onto a photodiode, such as a silicon photodiode. The resulting photocurrent may be amplified by a transimpedance amplifier whose voltage is then sampled by an analog-to-digital converter (ADC). Since the STAT system uses magnetic particles to pull the sample through the wax partition, a magnet such as a permanent magnet may be located at a bottom of the chamber. In other embodiments, the magnet may be movable by an actuator (e.g., stepper motor, solenoid, etc.) It may be advantageous to remove the magnetic field after the particles have moved through the wax in order to avoid clumping of the microbeads, which damages the reaction.

To remove the magnetic field, a controllable electromagnet may be used. In some embodiments, such an electromagnet may be too large and/or use too much power. In another embodiment, a permanent magnet is located at the bottom of the chamber and is moved away from the chamber after use (e.g., by way of a pivoting lever arm, translating rack, etc.) For example, a stepper motor may be controlled by the processor, which sends a move-to angle after the chamber has reached the first wax melting temperature for a certain period of time.

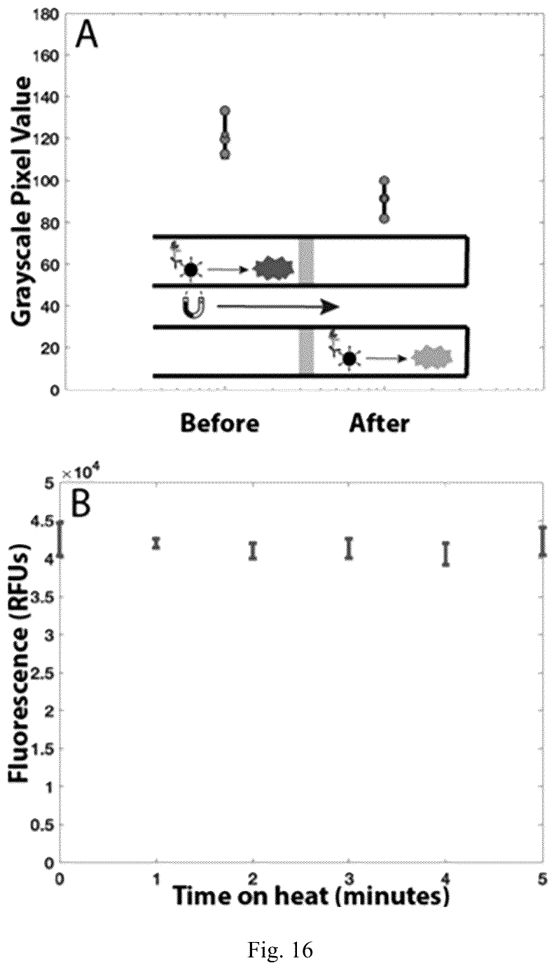

The STAT system may include a user GUI (see, for example, the GUI of the prototype embodiment shown in FIG. 16). For example, a Windows-based GUI may allow a user to start and record tests, change the heating profile parameters, and adjust the threshold value for what is considered a positive result. The device may have an indicator LED and start button. Once the various parameters are set using the GUI, they can be stored within the device (e.g., using a memory device in communication with the processor). In this way, a user has only to insert a sample cartridge, press the start button, and wait for the result to be displayed by the indicator (e.g., a color-coded indication (e.g., red, green) to provide a positive or negative result).

The terms “cartridge,” “sample cartridge,” “cassette,” and “sample cassette” are used interchangeably herein to describe the vessel for receiving a sample to be tested and may include reagents, TRAPs, microbeads, etc. Such sample cartridges may be removable from embodiments of the device. In this way, the device of embodiments of the present disclosure may be used for multiple tests, which may or may not be the same test. For example, the device may allow for changing a testing profile used to program the processor. In this way, the device may be used for different test types (e.g., different reagents, partition configurations, samples, etc.) In some embodiments, more than one testing profile may be stored within a memory of the device and the device may include a selector (e.g., button, dial, switch, touchscreen, etc.) for changing an active testing profile.

In some embodiments, the present disclosure may be embodied as a point-of-care testing device. The device includes a chamber configured to receive a sample cartridge. For example, a sample cartridge may be inserted and removed from the device and the chamber is configured to receive the sample cartridge. The device includes a heater such as, for example, a Peltier heater. The heater is configured to heat the sample cartridge. The heater may heat the ample cartridge directly or indirectly. For example, the heater may be integrated into the chamber to make contact with the sample cartridge. The heater may include one or more heating elements. The chamber may include a temperature sensor configured to measure a temperature of the sample cartridge and provide a temperature signal to the processor.

The device further includes an optical detector configured to detect an optical property of at least a portion of material within the sample cartridge. For example, the device may include an aperture configured to pass light from a portion of the sample cartridge (e.g., an aperture in the chamber or elsewhere). The optical detector may be, for example, a photodiode configured to detect light of a given wavelength—e.g., a fluorescent response, etc. For example, the detector may include a bandpass or other optical filter. In some embodiments, more than one optical detectors may be included. In such embodiments, each optical detector may be configured to detect a different property (e.g., a different wavelength, etc.)

The device includes a processor in electronic communication with the heater and the optical detector. The processor includes a memory for storing a testing profile. For example, the processor may be in electronic communication with a memory or may include such a memory with the processor. The processor is configured to operate the heater to heat the sample cartridge according to the testing profile. The processor is configured to receive a measurement signal from the optical detector at a predetermined time of the testing profile.

The device may include a light source configured to illuminate at least a portion of the material within the sample cartridge.

In some embodiments, the device further includes a sample cartridge. The sample cartridge may include a first reagent and a second reagent, where the first reagent and the second reagent are separated by a first meltable solid partition. The meltable solid partition may be a solid aliphatic partition, such as, for example, one or more alkanes (e.g., eicosane, docosane, hexacosane, heptacosane, nonococane, tetracosane, octadecane, etc.) The sample cartridge may be configured to receive a sample from a user and wherein the sample is in contact with the first reagent. The processor may be configured to heat the sample cartridge to a temperature sufficient to liquefy the first meltable solid partition at a predetermined time of the testing profile.

In some embodiments, the sample cartridge further includes a third reagent separated from the first reagent and the second reagent by a second meltable solid partition. The second meltable solid partition may be higher than a melting temperature of the first meltable solid partition. The second meltable solid partition may be lower than a melting temperature of the first meltable solid partition. The second meltable solid partition may be the same as a melting temperature of the first meltable solid partition.

The processor may be programmed to allow the sample cartridge to cool (e.g., stop heating) at a predetermined time of the testing profile.

In some embodiments, the device may include a magnet disposed adjacent to the chamber. An actuator may be included for moving the magnet from a first location to a second location. The processor may be further configured to move the magnet from the first location to the second location at a predetermined time during the testing profile. In embodiments having a magnet, a sample cartridge may include a first reagent and a second reagent, where the first reagent and the second reagent are separated by a first meltable solid partition, such as a solid aliphatic partition, and wherein a region of the sample cartridge containing the first reagent also includes a plurality of magnetic beads functionalized with capture molecules. The first position of the magnet may be such that the magnetic microbeads are urged through the first meltable partition. The second position of the magnet may be such that the magnet does not move the magnetic microbeads.

The testing profile of the processor may be configured to heat the sample cartridge to liquefy the first meltable partition and allow the magnetic microbeads to pass through the first meltable partition. The testing profile may be further configured to stop heating the sample cartridge for a predetermined period of time. The testing profile may be further configured to heat the sample cartridge to a second temperature to liquefy the second meltable partition and allow the second reagent and third reagent to mix; and receive a measurement signal from the optical detector at a predetermined time of the testing profile.

The following Statements provide various examples of the present disclosure.
Statement 1. A method for detecting the presence of an analyte comprising: i) contacting a sample suspected of containing the analyte together or separately with i) a plurality of magnetic beads having a plurality of first capture molecules attached thereto, wherein the first capture molecules have a specific affinity for the analyte, and ii) a plurality of second capture molecules having a detectable label attached thereto, wherein the second capture molecules have a specific affinity for the analyte, to form complexes, each complex comprising the analyte bound to the first capture molecule and the second capture molecule; ii) heating one or more solid aliphatic partitions to a temperature of 40° C. to 65° C., such that the one or more solid aliphatic partitions liquefy; iii) separating the complexes from unbound second capture molecules by selectively moving the complexes through the one or more liquefied aliphatic partitions via application of a magnetic field; and iv) detecting and optionally quantifying the signal generated from the detectable labels of the second capture molecules of the separated complexes; wherein the presence of a detectable signal is indicative of the presence of the analyte and the magnitude of the signal is indicative of the amount of the analyte in the sample.
Statement 2. A method according to Statement 1, wherein the sample is a biological sample.
Statement 3. A method according to Statement 3, wherein the biological sample is chosen from whole blood, blood fractions, plasma, serum, saliva, urine, stool, sweat, mucous, tears, breast milk, semen, tissue, placental tissue, conditioned medium, tissue culture medium, and bone marrow.
Statement 4. A method according to any one of the preceding Statements, wherein the analyte is an antibody or an antigen.
Statement 5. A method according to Statement 4, wherein the antibody is directed to a pathogenic antigen (e.g., a microbial antigen, a bacterial antigen, or a viral antigen).
Statement 6. A method according to Statement 5, wherein the viral antigen is associated with SARS-COV-2, Hepatitis C, Epstein-Barr, Zika, Ebola, Herpes simplex, Norovirus, Influenza, or Chikungunya.
Statement 7. A method according to any one of the preceding Statements, wherein the plurality of magnetic beads has a longest linear dimension of 100 nm to 50,000 nm.
Statement 8. A method according to any one of the preceding Statements, wherein the plurality of second capture molecules have horseradish peroxidase attached thereto.
Statement 9. A method according to any one of the preceding Statements, wherein the sample is sequentially contacted with the plurality of magnetic beads having the plurality of first capture molecules attached thereto and the plurality of second capture molecules having the detectable label attached thereto.
Statement 10. A method according to Statement 9, wherein the sample is rinsed after contacting the plurality of magnetic beads having the plurality of first capture molecules attached thereto and prior to contacting the plurality of second capture molecules having the detectable label attached thereto.
Statement 11. A method according to any one of the preceding Statements, wherein the one or more aliphatic partitions comprise one or more alkanes.
Statement 12. A method according to Statement 11, wherein one or more alkanes are chosen from eicosane, docosane, hexacosane, heptacosane, nonococane, tetracosane, octadecane, and combinations thereof.
Statement 13. A method according to any one of the preceding, wherein the magnetic field is generated by a magnet.
Statement 14. A method according to any one of the preceding, wherein the detectable signal is spectrophotometric (e.g., colorimetric or fluorescent).
Statement 15. A testing system comprising: a test assembly, the test assembly comprising an inlet configured to accept a sample comprising an analyte, a plurality of regions, and at least one solid aliphatic partition disposed therein, wherein adjacent regions of the plurality of regions are separated by one of the at least one solid aliphatic partition, and the plurality of regions includes a binding region and a detecting region, and the at least one solid aliphatic partition has a melting point of 40° C. to 65° C.; wherein the binding region comprises a plurality of magnetic beads having a plurality of first capture molecules attached thereto, wherein the first capture molecules have a specific affinity for the analyte and a plurality of second capture molecules having a detectable label attached thereto, wherein the second capture molecules have a specific affinity for the analyte, wherein the detecting region comprises a detection composition configured to enable spectrophotometric measurement of the composition; and a magnet configured to apply a magnetic field along a sequential path, whereby the plurality of magnetic beads move sequentially along the sequential path upon application of a magnetic force from the magnet, wherein the sequential path includes the binding region and the detecting region.
Statement 16. A testing system according to Statement 15, wherein the analyte is an antibody or an antigen.
Statement 17. A testing system according to Statement 16, wherein the antibody or the antigen is associated with SARS-COV-2, Hepatitis C, Epstein-Barr, Zika, Ebola, Herpes simplex, Norovirus, Influenza, or Chikungunya.
Statement 18. A testing system according to any one of Statements 15-17, wherein the at least one aliphatic partition comprises one or more alkanes.
Statement 19. A testing system according to Statement 18, wherein one or more alkanes are chosen from eicosane, docosane, hexacosane, heptacosane, nonococane, tetracosane, and octadecane.
Statement 20. A testing system according to any one of Statements 15-19, wherein the at least one aliphatic partition, the binding region, and the detecting region are arranged in a horizontal hydrophobic channel or a vertical hydrophobic channel.
Statement 21. A testing system according to any one of Statements 15-20, wherein the at least one aliphatic partition, the binding region, and the detecting region are arranged in a horizontal hydrophilic channel or a vertical hydrophilic channel.
Statement 22. A testing system according to any one of Statements 15-21, wherein the at least one aliphatic partition is configured to separate the binding region and the detecting region.
Statement 23. A testing system according to any one of Statements 15-22, wherein the binding region comprises a plurality of sub-regions, each adjacent region separated by one of the at least one aliphatic partitions.
Statement 24. The testing system according to Statement 23, wherein a first binding sub-region of the binding region comprises the plurality of magnetic beads attached to a plurality of first capture molecules, wherein the first capture molecule has a specific affinity for the analyte, and a second binding sub-region of the binding region comprises the plurality of second capture molecules attached to a detectable label, wherein the second capture molecule has a specific affinity for the analyte.
Statement 25. A testing system according to Statement 24, further comprising a rinsing sub-region disposed between the aliphatic partition of the first binding sub-region and the aliphatic partition of the second binding sub-region.
Statement 26. A testing system according to any one of Statements 15-25, wherein the magnet is external to the test assembly.
Statement 27. A testing system according to any one of Statements 15-26, wherein the magnet is configured to move across the test assembly along the sequential path.
Statement 28. A testing system according to any one of Statements 15-27, wherein the magnet is disposed on an end of the test assembly and the magnetic field extends across the binding region and the detecting region sequentially.
Statement 29. A method for detecting the presence of viral analyte comprising: i) contacting a sample suspected of containing the viral analyte with a plurality of magnetic beads having a plurality of capture molecules attached thereto, wherein the capture molecules have a specific affinity for the analyte to form complexes, each complex comprising the analyte bound to the capture molecule; ii) heating a first solid aliphatic partitions to a temperature of 35° C. to 45° C., such that the first solid aliphatic partition liquefies; iii) selectively moving the complexes through the liquefied aliphatic partition via application of a magnetic field; iv) contacting the complex with a protease such that viral genomic material is released from the viral analyte; v) heating the complex, the protease, and a second solid aliphatic partition to a temperature greater than or equal to 50° C., such that the protease deactivates and the second aliphatic partition liquefies; vi) contacting the viral genomic material with genomic amplification reagents; vii) amplifying the viral genomic material; viii) contacting the amplified viral genomic material with a detectable molecule; and ix) detecting and optionally quantifying a signal generated from the detectable molecule, wherein the presence of a detectable signal is indicative of the presence of the viral analyte and the magnitude of the signal is indicative of the amount of the viral analyte in the sample.
Statement 30. A method according to Statement 29, wherein the sample is whole blood, blood fractions, plasma, serum, saliva, urine, stool, sweat, mucous, tears, breast milk, semen, tissue, placental tissue, conditioned medium, tissue culture medium, or bone marrow. Statement 31. A method according to Statement 30, wherein the sample is whole blood. Statement 32. A method according to any one of Statements 29 to 31, wherein the viral analyte is a viral particle (e.g., virion).
Statement 33. A method according to any one of Statements 29 to 32, wherein the protease is Proteinase K.
Statement 34. A method according to any one of Statements 29 to 33, wherein the first aliphatic partition is eicosane.
Statement 35. A method according to any one of Statements 29 to 34, wherein the second aliphatic partition is hexacosane.
Statement 36. A method according to any one of Statements 29 to 35, wherein the viral analyte is associated with human immunodeficiency virus, SARS-COV-2, Hepatitis C, Epstein-Barr, Zika, Ebola, Herpes simplex, Norovirus, Influenza, or Chikungunya.
Statement 37. A method according to any one of Statements 29 to 36, wherein the melting point of the first aliphatic partition is lower than the melting point of the second aliphatic partition.
Statement 38. A method according to any one of Statements 29 to 37, wherein the first aliphatic partition has a longest linear dimension (e.g., thickness) that is larger than the longest linear dimension (e.g., thickness) of the second aliphatic partition.
Statement 39. A method according to any one of Statements 29 to 38, wherein when the second aliphatic partition liquefies, it results in addition of genomic amplification reagents to a mixture of the complex and viral genomic material.
Statement 40. A point-of-care testing device, comprising:
   a chamber configured to receive a sample cartridge;
   a heater configured to heat the sample cartridge;
   an optical detector configured to detect an optical property of at least a portion of a material within the sample cartridge; and
   a processor in electronic communication with the heater and the optical detector, the processor having a memory for storing a testing profile, and wherein the processor is configured to:
      operate the heater to heat the sample cartridge according to a testing profile; and receive a measurement signal from the optical detector at a predetermined time of the profile.
Statement 41. A device according to Statement 40, wherein the chamber includes a temperature sensor configured to measure a temperature of the sample cartridge and provide a temperature signal to the processor.
Statement 42. A device of according to Statement 40 or Statement 41, further comprising a light source configured to illuminate at least a portion of the material within the sample cartridge.
Statement 43. A device according to any one of Statements 40-42, wherein the optical detector is configured to detect fluorescence.
Statement 44. A device according to any one of Statements 40-43, further comprising a sample cartridge, the sample cartridge including a first reagent and a second reagent, where the first reagent and the second reagent are separated by a first meltable solid partition, such as a solid aliphatic partition.
Statement 45. A device according to Statement 44, wherein the sample cartridge is configured to receive a sample from a user and wherein the sample is in contact with the first reagent.
Statement 46. A device according to Statement 44 or Statement 45, wherein the processor is configured to heat the sample cartridge to a temperature sufficient to liquefy the first meltable solid partition at a predetermined time of the testing profile.
Statement 47. A device according to any one of Statements 44-46, wherein the meltable solid partition is a solid aliphatic partition.
Statement 48. A device according to Statement 47, wherein the solid aliphatic partition comprises one or more alkanes (e.g., eicosane, docosane, hexacosane, heptacosane, nonococane, tetracosane, octadecane, etc.)

Statement 49. A device according to any one of Statements 44-48, wherein the sample cartridge further includes a third reagent separated from the first reagent and the second reagent by a second meltable solid partition.
Statement 50. A device according to Statement 49, wherein a melting temperature of the second meltable solid partition is higher than a melting temperature of the first meltable solid partition.
Statement 51. A device according to Statement 49 or Statement 50, wherein a melting temperature of the second meltable solid partition is lower than a melting temperature of the first meltable solid partition.
Statement 52. A device according to any one of Statements 49-51, wherein the processor is programmed to allow the sample cartridge to cool at a predetermined time of the testing profile.
Statement 53. A device according to any one of Statements 40-52, further comprising: a magnet disposed adjacent to the chamber; and an actuator for moving the magnet from a first location to a second location.
Statement 54. A device according to Statement 53, wherein the processor is further configured to move the magnet from the first location to the second location at a predetermined time during the testing profile.
Statement 55. A device according to Statement 53 or Statement 54, further comprising a sample cartridge, the sample cartridge including a first reagent and a second reagent, where the first reagent and the second reagent are separated by a first meltable solid partition, such as a solid aliphatic partition, and wherein a region of the sample cartridge containing the first reagent also includes a plurality of magnetic beads functionalized with capture molecules.
Statement 56. A device according to Statement 55, wherein the first position of the magnet is such that the magnetic microbeads are urged through the first meltable partition.
Statement 57. A device according to Statement 55 or Statement 56, wherein the second position of the magnet is such that the magnet does not move the magnetic microbeads.
Statement 58. A device according to any one of Statements 54-57, wherein the sample cartridge further comprises a third reagent separated from the second partition by a second meltable solid partition.
Statement 59. A device according to Statement 58, wherein the second partition has a melting point higher than a melting point of the first partition.
Statement 60. A device according to Statement 59, wherein the testing profile of the processor is configured to:
   heat the sample cartridge to liquefy the first meltable partition and allow the magnetic microbeads to pass through the first meltable partition;
   stop heating the sample cartridge for a predetermined period of time;
   heat the sample cartridge to a second temperature to liquefy the second meltable partition and allow the second reagent and third reagent to mix; and
   receive a measurement signal from the optical detector at a predetermined time of the testing profile.
Statement 61. A device according to any one of Statements 40-60, further comprising a battery.
Statement 62. A device according to any one of Statements 40-61, further comprising a user input (e.g., button, trigger, switch, etc.) and the processor is further configured to begin the testing profile upon receiving a first signal from the user input.
Statement 63. A device according to any one of Statements 40-62, further comprising an indicator (e.g., one or more LEDs, multi-color LED, etc.) and the processor is further configured to provide a result signal to the indicator based on the received measurement signal.
Statement 64. A device according to any one of Statements 40-63, further comprising a programming interface for communication with an external system, and wherein the processor is configured to receive a testing profile from the external source by way of the programming interface and to store the testing profile in the memory.

The following examples are provided as illustrative examples and are not intended to be restrictive in any way. These examples provide desired parameters for the method and the test system.

### Example 1

Described are the results of tests performed to determine the geometric parameters of the test assembly in a preferred example. The term “TRAP” or “TRAPs” may be used interchangeably with the terms “aliphatic partition” or “thermally responsive aliphatic partitions” or “alkane partition” or “wax partitions” or “eicosane partitions.”

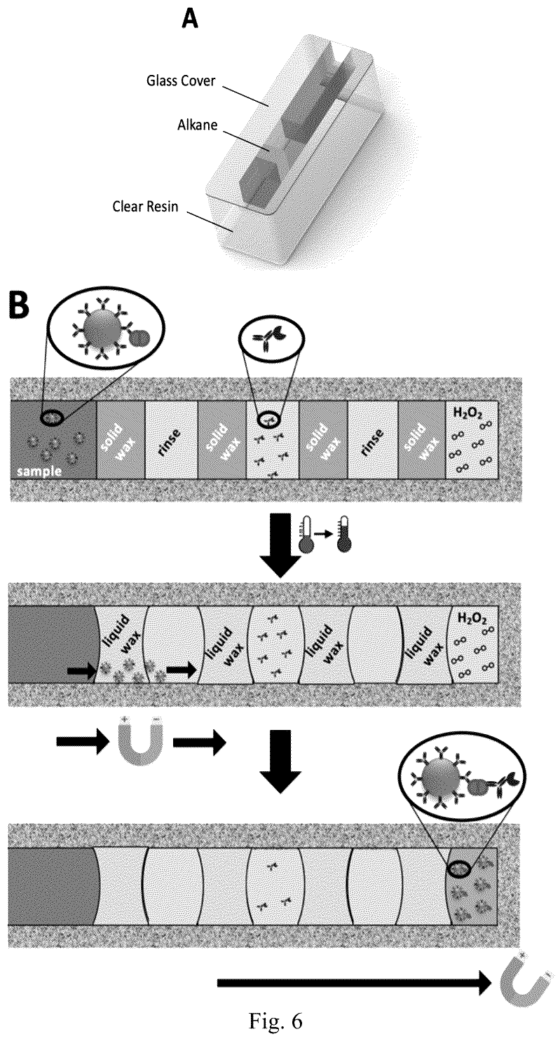

A flexible form of a TRAP in which liquefied partitions remain in place and continue to separate reagents while magnetic beads can be pulled through the liquefied partitions in a magnetofluidic assay is shown in FIG. 6. FIG. 6A depicts a 3D representation of a device with an alkane TRAP partitioning two regions comprising liquids. FIG. 6B is a schematic illustration of an immunoassay using TRAPS, in which alkane wax layers partition all reagents. When warmed, the partitions liquefy, enabling magnetic beads to be pulled from one region (e.g., reagent zone) to the next, enabling sample-to-answer assays. The alkane partitions can continue to separate reagents when confined within sufficiently small geometries. By using phase-changing alkanes as partitions, the reagents can remain partitioned even during shipping and handling, while magnetofluidic manipulation of reagents across partitions can be conducted following thermal actuation.

Described herein is the design and rules that dictate whether the partition is removed for reagent addition or remains stationary for continual partitioning. Because of the density and polarity differences between the alkane and the aqueous reagents, the behavior is dependent upon the surface energy of the reaction vessel and the vessel orientation (i.e., vertical versus horizontal). This example describes the design rules for all permutations of these conditions. The design rules for pulling magnetic microbeads through liquefied partitions without causing reagent breaches was investigated.

All of the test assemblies tested in this example were 3D-printed with resin from Formlabs. The cover slips were obtained from Fisher Scientific. The blue and yellow dyes were purchased from Wilton Color Right and were used to color water. The alkane used in the experiments described herein was n-eicosane (melting point 42° C.), 99%, purchased from Alfa Aesar. Streptavidin magnetic beads (1.05 μm diameter) were purchased from BioLabs. The glue used to adhere glass to resin was Scotch liquid super glue. Heat-inactivated fetal bovine serum (FBS) from American Type Culture Collection (ATCC) was used as a means to hydrophilize the surfaces of the test assemblies. Amplex Red was purchased from Biotium, and the hydrogen peroxide used to react with Amplex Red was from Fisher Scientific. The biotinylated rabbit IgG antibody and the HRP-conjugated anti-rabbit IgG antibody were both manufactured by ThermoFisher. The bead wash buffer was made with Tris and NaCl, both from Sigma Aldrich. Finally, the phosphate buffer was made with components from JT Baker.

Test assemblies with various channel geometries were fabricated of resin 3D printed by a Form 2 stereolithography printer (Formlabs). The parts were cleaned with isopropyl alcohol (IPA) to ensure no uncured resin remained on the printed test assembly. The outer dimensions of each test assembly were the same: 10 mm wide, 8 mm in height, and 25 mm long. There were five different channel geometries used in this example, all were 22 mm long: 2×2 mm, 3×3 mm, 4×4 mm, 4.5×4.5 mm, and 5×5 mm. One 22 mm long face of these inner dimensions was open to air when printed and was covered by a glass cover slip, 10 mm wide, 25 mm long, and 0.15 mm thick, and glued in place. Some channels were modified to be hydrophilic. These hydrophilic channels were filled with FBS and soaked at room temperature for 2 hours to increase hydrophilicity.

To quantify the hydrophobicity of the resin material in these experiments, the contact angle of a drop of water on a resin slab was measured. This was done by placing the slab and a drop of water onto a contact angle goniometer and recording the resulting angle. The contact angle of the untreated resin was found to be 82.0° (S.D.=2.8°, n=5), while the contact angle of the FBS-treated resin was measured as 38.8° (S.D.=8.3°, n=5), indicating an increase in hydrophilicity.

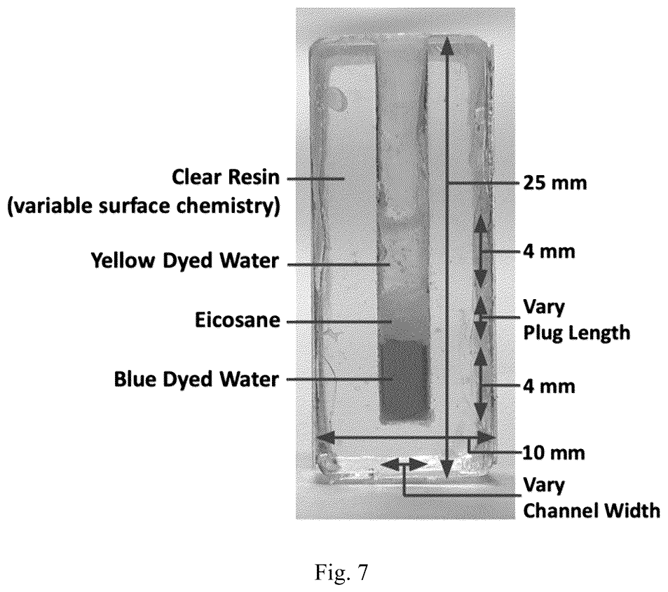

To investigate the behavior of TRAPs in various geometries, the channels were filled with two layers of water separated by a TRAP. First, channels were filled with the respective volume of blue dyed water to fill 4 mm along the channel, as shown in FIG. 7. FIG. 7 is a labeled photograph of a TRAP with yellow dyed water on top of eicosane on top of blue dyed water. To deposit eicosane, it was first melted and then pipetted on top of the water. Eicosane was melted by placing it in a glass vial on a hot plate at 120° C. Melted eicosane quickly hardened when deposited onto the water, sealing the blue dyed water layer. Yellow dyed water was pipetted on top of the hardened eicosane. The volume of yellow dyed water equaled that of the blue dyed water, except for the case of the 5×5 mm channels, for which the volume of yellow dyed water was doubled to ensure the eicosane layer never contacted air when the channel was positioned horizontally. Once filled, each test assembly was placed on a 60° C. hot plate either horizontally (resting on the 25×10 mm resin face) or vertically (resting on the 10×8 mm resin face). After about 3 minutes, the eicosane melted and was either breached, allowing the two dyes to mix, or remained in place, preventing the two dyes from mixing. A breach is defined by green color appearing in the region near the TRAP or if any part of the TRAP was detached from the channel walls. The volume of eicosane used was experimentally varied to observe which plug lengths (calculated by dividing the volume of eicosane by the channel cross-sectional area) caused a TRAP to be breached or to stay in place.

To investigate the potential to use TRAPs in magnetofluidic methods, the stability of stationary TRAPs and the leakage of TRAPs as magnetic beads are pulled through the liquefied partitions was tested. Water was placed in a 3×3 mm channel such that 4 mm of the channel was filled (36 μL). 18 μL of melted eicosane was placed on top of the water to fill another 2 mm of the channel. 36 μL of a solution containing 10 μM FAM fluorescein and 40 μg magnetic beads in water was then placed on top of the eicosane layer. The peak absorbance wavelength of FAM was 495 nm, while the peak emission wavelength was 520 nm. After the channel was set up, fluorescence measurements of both sides of the TRAP were taken. The channel was then placed on a 60° C. hot plate. Once the eicosane melted, the magnetic beads were gathered against the glass cover by holding a neodymium magnet against the glass on the outside of the channel. The magnet was then moved along the glass to the other side of the TRAP at about 2 mm/s, pulling the beads along with it. The magnet was removed and the channel was taken off the hot plate. Then, once the eicosane re-hardened, another set of fluorescence measurements of both sides of the TRAP was taken.

To study the geometry constraints to prevent leakage, blue dyed water was placed into a 3×3 mm channel such that 4 mm of the channel was filled up (36 μL). Melted eicosane was placed on top of the water. Yellow dyed water with magnetic beads filled 8 mm of the channel on top of the eicosane (72 μL). The channel was then placed on a 60° C. hot plate. Once the eicosane melted, the magnetic beads were gathered against the glass cover by holding a neodymium magnet against the glass on the outside of the channel. The magnet was then moved along the glass to the other side of the TRAP, pulling the beads along with it. The magnet was removed and the channel was taken off the hot plate. The TRAP was classified as “bridged” if color mixing was observed or if the eicosane had separated from the glass surface. The amount of eicosane and magnetic beads were experimentally varied to determine which combinations of partition thickness and bead mass caused the TRAP to bridge or remain intact.

To investigate if the magnetofluidic method detailed in the present disclosure could be implemented in an immunoassay, tests were performed to determine whether antibodies captured on streptavidin magnetic beads could be transferred across one, two, and three layers of melted TRAPs without a significant amount dissociating from the beads. 1 mg of the streptavidin-coated magnetic microbeads was incubated with 35 μg biotinylated rabbit IgG antibody in solution for 90 minutes at room temperature. They were then washed by gathering them to the side of the tube, aspirating out the supernatant, and rinsing them with 200 μL of 25 mM Tris and 150 mM NaCl buffer three times. After the final rinse, 56 μg of HRP-conjugated anti-rabbit IgG antibodies in phosphate buffer were added to the mass of beads and incubated for 10 minutes at room temperature (from 20° C. to 25° C.). Then the wash step was repeated, except after the final rinse, 60 μL of phosphate buffer was added to the mass of beads. 1 mg of streptavidin-coated magnetic microbeads was rinsed three times with 200 μL of 25 mM Tris and 150 mM NaCl buffer and put into 60 μL of phosphate buffer. 56.4 μL of this batch of beads were added to 0.6 μL of the antibody-bound beads for a 1:100 dilution of the antibody-bound beads. A 30 μL solution of 0.05 mM Amplex Red and 1 mM hydrogen peroxide was added to the bottom of a 3×3×42 mm channel. Then, 30 μL of melted eicosane were placed on top of the solution. Three sets of three channels were designated to represent three different scenarios: magnetic beads travelling through one, two, and three layers of melted TRAPs. In the first set, 26 μL of phosphate buffer and 4 μL of the prepared 1:100 antibody-bound magnetic bead solution were placed on top of the eicosane layer. In the second set, a 50 μL phosphate buffer rinse layer was placed on top of the eicosane, followed by 30 μL of melted eicosane. Then 26 μL of phosphate buffer and 4 μL of the prepared 1:100 antibody-bound magnetic bead solution were added. In the third set, a 50 μL phosphate buffer rinse layer was placed on top of the eicosane followed by 30 μL of melted eicosane, then 50 μL of phosphate buffer, then 30 μL of melted eicosane. Finally, 26 μL of phosphate buffer and 4 μL of the prepared 1:100 antibody-bound magnetic bead solution were added. In each case, the test assemblies were placed on a 60° C. hot plate to melt the eicosane layers. Once the eicosane was melted, the magnetic beads were gathered against the glass cover by holding a neodymium magnet against the glass on the outside of the channel. The magnet was moved along the glass at a rate from 0.2 mm/s to 10 mm/s to the Amplex Red and hydrogen peroxide layer, pulling the beads along with it. The magnet was removed and the channel was taken off the hot plate. 10 minutes later, a fluorescence measurement of the final layer was taken. As a no-TRAP control, three additional channels were prepared where 4 μL of the prepared 1:100 antibody-bound magnetic bead solution were placed at the bottom of the channels, a magnet was applied to pull the beads to a side of the channels, the supernatant was aspirated out, and 30 μL of the 0.05 mM Amplex Red and 1 mM hydrogen peroxide solution were added to the beads. 10 minutes later, a fluorescence measurement of the reaction was taken.

All fluorescence measurements were taken with a florescence plate reader, and the results are shown in FIGS. 9-12. All fluorescence measurements were taken by placing a device into a portable fluorescence reader as shown in FIG. 8. The device comprises a 3D printed support that houses an ArduCAM MT9M001 Camera with an ArduCAM USB2 Camera Shield (ArduCAM), a longpass filter (Thorlabs) on the lens of the camera (530 nm for the FAM fluorescein readings or 570 nm for the Amplex Red readings), two LEDs to excite the sample (470 nm blue for the FAM fluorescein readings or 525 nm green for the Amplex Red readings), and a support that secures the channels in a precise location relative to the camera.

The observations using alkane to initially separate assay reagents in microtubes showed that wax liquefaction led to partition breach in which reagents could automatically be added and mixed on-demand. In a narrower channel, surface tension and the hydrophobic interactions between wax and resin could keep the liquefied alkane partition in place despite the density difference between it and the surrounding liquids.

In this example, geometric design rules were determined, mathematically and experimentally, that govern when the liquefied alkane barrier continues to partition and when it breaches, promoting reagent mixing.

FIG. 9 shows the results of a vertically oriented channel with a hydrophobic channel wall. FIG. 9A displays a photograph of a melted TRAP in a vertical hydrophobic channel. FIG. 9B displays a 2-dimensional schematic of a cross-section along the center of the channel parallel to the image plane in FIG. 9A before and after melting the TRAP. FIG. 9C displays data representing the TRAPs that remained intact (circle) and TRAPs that breached (triangle), as well as the threshold (dashed line) determined by a mathematical approximation.

It was observed that because of the hydrophobic surface, the liquefied eicosane formed a concave meniscus at each aqueous interface as shown in FIG. 9B. The angle of the water-eicosane boundary against the wall at a corner was measured to be 45° with a standard deviation of 5.9°. Because of the meniscus shapes, it was concluded that as the partition thickness (P) decreases, the thickness of the partition at the center point will reach zero for a non-zero partition thickness, thus causing a breach in the eicosane plug and allowing the two initially separated layers to mix.

To mathematically predict the threshold partition length (Pₜₕ) that determines whether a breach will occur, the following assumptions were made: (1) the meniscus at a water-eicosane interface is a portion of the surface of a sphere constrained by the square cross-section of a channel whose centerline includes the center of the sphere (see dashed curve in FIG. 9B); and (2) breaching occurs (e.g., only occurs) when the two meniscuses touch along the centerline of the channel (i.e. when Pₙ−2h=0, where Pₙ is the distance between the two meniscuses at a corner of the channel and h is the depth of a meniscus at the centerline of the channel depicted in FIG. 9B).

Equation 1 dictates how the threshold value of the partition thickness Pₜₕ varies with respect to the channel width D and contact angle θ, which is the angle between the eicosane and water at a corner of the channel (FIG. 9B). It is important to note that the cross-section schematic shown in FIG. 9B is parallel to the image plane in FIG. 9A. Because the angle calculations for 0 were done at the corners of the channel, Equation 1 operates along the diagonal of the channel, and thus √2 D is used.

$${P}_{th} = \sqrt{2} D \frac{1 - sin ( θ )}{cos ( θ )} - \frac{π}{6 \sqrt{2} D} \times \frac{1 - sin ( θ}{cos ( θ )} [ {( \frac{3}{4} \sqrt{2} D )}^{2} + {( \frac{1}{2} \sqrt{2} D )}^{2} + {( \frac{1}{2} \sqrt{2} D \frac{1 - sin ( θ )}{cos ( θ )} )}^{2} ] \qquad ( 1 )$$

The data from the experiment confirmed the predictive ability of the mathematical derivation. As shown in FIG. 9C, when an eicosane partition in the channel has a partition thickness below the estimated threshold, the melted eicosane layer breaches and allows the liquids to mix (FIG. 9C triangles). When the partition thickness is above the estimated threshold, the melted eicosane remains intact and no mixing occurs (FIG. 9C circles). Because the TRAPs remain intact at relatively small partition thicknesses, the vertical hydrophobic channel appears to be suitable for applications for partitioning reagents in sample-to-answer magnetofluidic immunoassays.

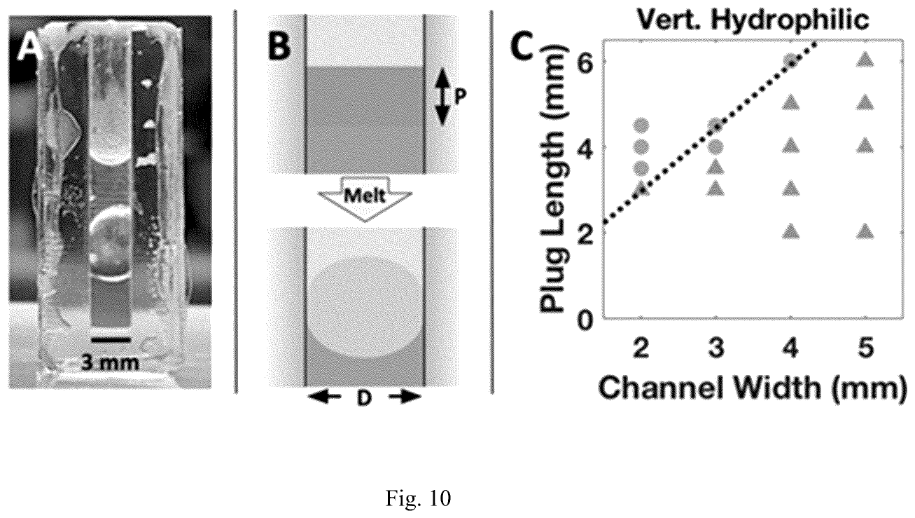

FIG. 10 shows the results of a vertically oriented channel with a hydrophilic channel wall. FIG. 10A displays a photograph of a melted TRAP in a vertical hydrophilic channel. FIG. 10B displays a 2-dimensional schematic of a cross-section along the center of the channel parallel to the image plane in FIG. 10A before and after melting the TRAP. FIG. 10C displays data representing the TRAPs that remained intact (circle) and TRAPs that breached (triangle), as well as the threshold (dashed line) determined by a mathematical approximation.

It was demonstrated that TRAPs can maintain partitioning capability in a vertical channel despite gravity, due to surface tension and the hydrophobic interactions between wax and resin. Next, the behavior of the system when the channel has a hydrophilic wall surface was investigated. In this case, the hydrophilic wall caused liquid wax to separate off of the wall, especially at thinner partition thicknesses (FIG. 10). This enabled breaching along the corners of the channel and ultimately led to floating wax in many cases.

To mathematically predict the threshold that determines whether a breach will occur given a certain partition thicknesses and square channel width, the following assumptions were made: (1) the wax will form a sphere and float up through the water with no interference from channel walls; and (2) with interference from channel walls, the wax will remain (e.g., only remain) in place as a partition when the radius of the sphere it would become given the input volume equals the length from the center of the channel to a corner. Equation 2, which determines the threshold partition length at which a TRAP will breach, is derived by equating the threshold input volume (D×D×Pₜₕ) with the assumed spherical shape that forms when the wax melts and attempts to break free of the channel walls. The threshold Pₜₕ is plotted as a dashed black line in FIG. 9C.

The experiments confirm the predictive capability of this equation. Most combinations of partition thickness and channel width that were below the threshold line resulted in the mixing of the two initially partitioned liquids (FIG. 10C triangle). Likewise, most combinations of partition thickness and channel width that were above the threshold line resulted in a stationary partition that prevented the two liquids from mixing. Given that the hydrophilic case results in breaching for even moderately thick partitions, this arrangement appears to be suitable for applications in which the partition is to be removed in order to automate precise reagent additions and mixing.

$${P}_{th} = \frac{\sqrt{2}}{3} π D \qquad ( 2 )$$

FIGS. 9 and 10 demonstrate the impact of surface energy on the behavior of the liquefied partition. Because gravity also plays an important role in the behavior of the liquefied partitions, the above investigations were repeated for a channel placed horizontally.

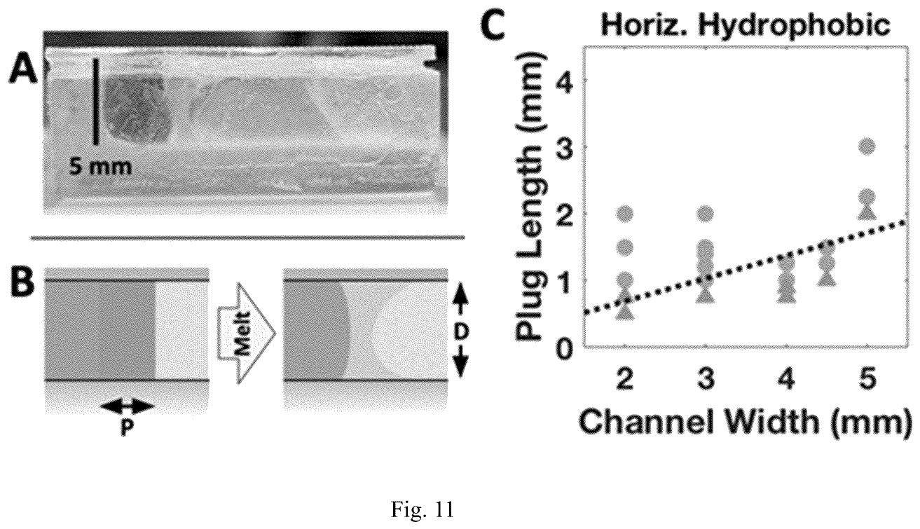

FIG. 11 shows the results of a horizontally oriented channel with a hydrophobic channel wall. FIG. 11A displays a photograph of a melted TRAP in a horizontal hydrophilic channel. FIG. 11B displays a 2-dimensional schematic of a cross-section along the center of the channel parallel to the image plane in FIG. 11A before and after melting the TRAP. FIG. 11C displays data representing the TRAPs that remained intact (circle) and TRAPs that breached (triangle), as well as the threshold (dashed line) determined by a mathematical approximation.

As shown in FIG. 11, the meniscuses observed in the vertical hydrophobic case were present again. However, under the influence of gravity, these meniscuses were asymmetric, with more wax along the top wall of the channel, especially in larger channel widths (FIGS. 11A and 11B). Thus, it can be concluded that when the partition thickness is thin enough, the breach will still occur near the center of the channel. Because the breaching behavior was similar to the vertical hydrophobic case and since the asymmetry due to gravity is not as prevalent in smaller channel widths, the same mathematical analysis as in the vertical hydrophobic case was used (Equation 1).

As expected, this model matched with the data at smaller channel widths but is less accurate when predicting the behavior at larger channel widths (>3 mm, FIG. 11C). The experimental results demonstrate that the horizontally oriented hydrophobic channel may be a viable system for continually partitioning reagents in a magnetofluidic assay.

FIG. 12 shows the results of a horizontally oriented channel with a hydrophilic channel wall. FIG. 12A displays a photograph of a melted TRAP in a horizontal hydrophilic channel. FIG. 12B displays a 2-dimensional schematic of a cross-section along the center of the channel parallel to the image plane in FIG. 12A before and after melting the TRAP. FIG. 12C displays data representing the TRAPs that remained intact (circle) and TRAPs that breached (triangle), as well as the threshold (dashed line) determined by a mathematical approximation.

The horizontal hydrophilic channels resulted in similar wax behavior as the vertical hydrophilic case in which the wax tended to detach from the channel walls and form a spherical shape when subjected to no external interference. However, because in that situation gravity was in a perpendicular direction relative to the length of the channels, the sphere of wax floated up against the surface of the glass cover, which resulted in the formation of the bulged shape shown in FIG. 12A. It was observed that the wax sealed the channel when this bulged shape pressed against the side walls of the channel such that the wax reached all corners of the channel. At this point, the wax in contact with a side wall formed a near-semicircular shape. Because of this, the mathematical analysis estimated the volume of the wax at the threshold as the area of a semicircle (diameter d equals the channel width D) times the channel width with an adjustment factor, a, which modifies the equation to account for deviations in the cross-section from a perfect semicircle across the width of a channel. The empirically determined term a is necessary because of the combinations of partition thicknesses and channel widths that result near the threshold of a breached TRAP. In this domain, the force of the eicosane surface tension cause a liquid bridge to form that extends the eicosane semicircle shape towards the wall to seal the channel, though the geometry would result in a semicircle with a radius smaller than the width of the channel. Equating this formula to the volume of the input wax (D×D×P) and setting α=0.9 (determined empirically) results in Equation 3, plotted as a dashed line in FIG. 12C. The data matches to this mathematical approximation, and each TRAP above the threshold line remained intact (FIG. 12C circles) and most points below the line allowed the liquids to mix (FIG. 12C triangles).

As with the vertical hydrophilic channels, the experimental results demonstrate that the horizontal hydrophobic channels result in relatively unstable partitions. Thus, this arrangement may be viable for applications in which the partition must be removed in order to automate precise reagent additions and mixing.

$${P}_{th} = α \frac{π}{2} D \qquad ( 3 )$$

The experiments above (FIGS. 9 and 11) demonstrate that when confined in small hydrophobic channels, the TRAPs remain stationary upon liquefaction. It was hypothesized that the liquefied partitions would be permeable to magnetic beads driven by an external magnet and would remain stationary as the beads are pulled through, thus continuing to partition the solutions while the beads are transferred from one solution to the other. To verify this capability, a channel in which a TRAP partitioned water and an aqueous solution with the fluorophore FAM was tested. The magnetic beads were also loaded into the FAM solution. A fluorescence reader (shown in FIG. 8) was used to measure the fluorescence on both sides of the partition before and after the magnetic beads were pulled through the TRAP. As shown in FIG. 13, the beads were pulled from one side of the partition to the other without disturbing the partition. The beads were pulled using an external magnet moved from 0.2 mm/s to 10 mm/s. Furthermore, no fluorescence change was measurable on the destination side of the partition, suggesting that the TRAP continued to partition during the bead transfer.

Specifically, FIG. 13A is a schematic depicting a fluorescence measurement taken before and after magnetic beads were transferred across a TRAP. FIG. 13B displays data showing fluorescence values of the FAM side (green) and water side (blue) of the TRAP.

While the system in FIG. 13 shows continued partitioning, it was hypothesized that if the partition length was short enough and if the bead cluster volume was large enough, bridging between the partitioned zones could occur (FIG. 14A). Thus, the geometric design rules must also account for this possibility. To investigate this, the partition thickness and the bead volume was varied, and it was determined which conditions resulted in breaching of the partition. The data in FIG. 14 demonstrates that thinner partitions and larger quantities of beads led to bridging (triangles). Bridging occurs when the cluster of beads that is pulled across a TRAP connects the two liquids on either side. When this occurs, the TRAP does not reseal once the two aqueous layers are bridged.

Specifically, FIG. 14A displays a schematic depicting that fewer beads and a thicker TRAP (top of figure) will not bridge, while more beads and a thinner TRAP (bottom of figure) may bridge, causing the two liquids to breach. FIG. 14B displays data showing which experimental combinations of plug length and bead mass caused the TRAPs to remain intact (circles) or bridge (triangle). FIG. 14C displays a photograph of 64 μg of magnetic beads traveling through a 2 mm TRAP plug length with no TRAP bridging. The top of FIG. 14C shows the magnetic beads before movement, and the bottom of FIG. 14C shows the magnetic beads after movement via a magnetic field.

FIG. 15 displays the fluorescence signals of the antibody-bound beads that traveled through one, two, or three TRAPs, and a no target control (NTC) that included beads with no antibodies that traveled though one TRAP (n=3). It was hypothesized that magnetic beads functionalized with proteins that can capture biomarkers are capable of transferring biomarkers across liquefied TRAPs. This step is necessary in common immunoassays in which biomarkers or labeling antibodies are bound in one step and then transferred to a rinse step and subsequent binding steps. Because the temperature and hydrophobicity of the wax could cause antibodies and antigens to denature, the binding stability was assessed as the antibody complexes were carried through a TRAP via a magnetic bead. HRP-conjugated anti-rabbit IgG antibodies were used as a model antigen (i.e., captured biomarker), captured by rabbit IgG. The antibodies attached to the magnetic beads to form a sandwich. The bound complex was transferred across different numbers of TRAPs and loss of binding was quantified (using Amplex Red and hydrogen peroxide on the destination side of the TRAP). The data in FIG. 15 demonstrates no significant difference between fluorescence signals of the antibody-bound beads after travelling through a single TRAP and through subsequent numbers of TRAPs. Since a substantial amount of signal is seen after the antibody-bound beads are transferred (compared to the no target control), it was concluded that the TRAPs are compatible with immunoassays.

### Example 2

The following example provides examples of methods and devices of the present disclosure.

To maximize access to SARS-COV-2 serological (antibody) tests, point-of-care (PoC) options are used. PoC tests require sample-to-answer functionality, which is challenging with whole blood. This example demonstrates a sample-to-answer SARS-COV-2 antibody test from whole blood using automated thermally actuated valves. Higher-order alkanes serve as partitions between immunoassay regions (e.g., zones (sample/bind, rinse, detection)); upon warming, the partitions liquefy, enabling magnetic beads to be moved through each zone while continuing to partition the reagents. The instant data show a detection limit of 0.7 ng/mL SARS-COV-2 antibodies, multiple orders of magnitude lower than clinically relevant concentrations.

This example displays a SARS-COV-2 serological test from whole blood that eliminates precise manual steps without the need for equipment. As shown in FIG. 1, assay reagent regions are initially partitioned using eicosane, a solid wax at ambient temperature; when warmed, the wax liquefies. As depicted in Example 1, it was found that when confined in a narrow channel, the liquefied partitions remain in place, but allow magnetic beads to be pulled from one region to another by an external magnet, enabling the wax to be used as permeable partitions. In this sample-to-answer assay, beads functionalized with SARS-COV-2 spike protein capture patient antibodies from the blood sample (along with a labeling antibody to complete the sandwich assay) and are then pulled through liquefied partitions, first into a rinse region, then into a detecting region.

Described are the results of SARS-COV-2 antibody detection utilizing the thermally responsive aliphatic partition and magnetic bead combination, as described in the present disclosure. This testing method separated reagent compositions into three regions/sub-regions, a binding region, a rinse region, and a detection region by alkane partitions made of eicosane wax. These alkane partitions continued to separate reagent compositions in the binding region, the rinse region, and the detection region while in a solid state at ambient temperature and while in a liquefied state after heating the alkene partitions to 42° C.

As shown in FIG. 1, the binding region housed magnetic beads functionalized with SARS-COV-2 spike proteins and horseradish peroxidase labeled (HRP-labeled) antibodies. When a whole blood sample was added to this zone, SARS-COV-2 antibodies present in the sample bound to the magnetic beads, while HRP-antibodies in solution labeled the SARS-COV-2 antibodies to form a sandwich.

After a 30-minute incubation period, the alkane partitions were warmed to 42° C., causing the alkane partitions to become liquefied. Once liquefied, an external magnet was moved in the direction from the binding compartment to the rinsing compartment. The movement of the external magnet in this direction pulled the magnetic beads with the antibody sandwiches into the rinse compartment to rinse the bound sandwiches from unbound magnetic beads and unbound HRP-labeled antibodies.

The external magnet was then moved in the direction from the rinse region to the detecting region. The movement of the external magnet in this direction pulled the magnetic beads with the antibody sandwiches into the detecting region containing Amplex Red and H₂O₂. The HRP-labeled antibody within the sandwich reacted with the Amplex Red and the H₂O₂, to convert Amplex Red into a fluorescent product. The detectable fluorescence was proportional to the concentration of SARS-COV-2 antibodies, quantifiable in a custom portable reader that contains an integrated nichrome heater, an LED, and an Arducam camera.

In this example, the impact of both the eicosane wax and heat on the stability of the antibody sandwiches was assessed. To do this, antibody sandwiches bound to the magnetic beads were pulled across one alkane partition and into a second region containing Amplex Red and H₂O₂, via a magnetic field of an external magnet. The results are shown in FIG. 16A. As shown in this figure, the resulting fluorescence in the detection zone was 76% of the total fluorescence produced from the bead sandwiches initially added, suggesting that the majority of the labeling antibodies remain bound to the beads and crossed the alkane partition. Additionally, the stability of the antibody sandwiches when exposed to 60° C. was tested, as shown in FIG. 16B. This temperature is significantly higher than the melting temperature of the eicosane. To do this, a sandwich was formed with beads functionalized with spike protein, an anti-spike-protein antibody, and an HRP-labeled secondary antibody. The beads were heated in solution, and then the amount of HRP bound to the beads was assessed using Amplex Red. There was no significant loss of bound antibodies in 5 minutes, demonstrating that the warming step to melt the eicosane will not negatively affect the assay performance.

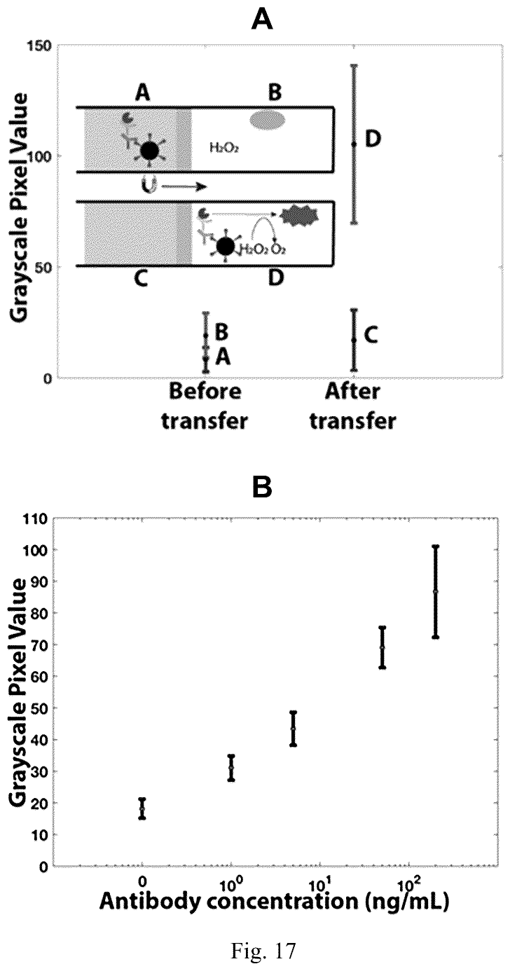

Further, in this example, the critical step of binding the antibodies from whole blood, along with a labeling secondary antibody, and removing them while keeping the blood separate was assessed. FIG. 17A demonstrates that the spike-functionalized magnetic beads were able to capture SARS-COV-2 antibodies (spiked into commercially purchased human blood) and HRP-labeled antibodies that could be pulled into a detecting region with Amplex Red via the magnetic field of an external magnet. No visible blood was observed crossing into the detecting region.

The concentration detection of SARS-COV-2 antibodies was assessed. As shown in FIG. 17B, in this example, the detection limit was 0.7 ng/ml, which is about 100 times lower than patient samples though previously tested positive for SARS-COV-2.

This example demonstrates a sample-to-answer method for detection of SARS-COV-2 antibodies with a limit of detection of 0.7 ng/mL. The detection limit is also comparable to other proposed PoC systems for SARS-COV-2 antibody detection, yet does not require external or manual blood preparation.

This method for serological detection of SARS-COV-2 uses alkane partitions to integrate blood preparation steps into a PoC platform for true sample-to-answer diagnosis. Also demonstrated was the detection of SARS-COV-2 antibodies from whole blood at concentrations well below physiological relevance without the need for precise manual steps or equipment.

### Example 3

The following example provides examples of methods and devices of the present disclosure.

Described are the results of SARS-COV-2 antibody detection utilizing the thermally responsive aliphatic partition and magnetic bead combination, as described in the present disclosure. FIG. 3 demonstrates this sample-to-answer SARS-COV-2 spike protein antibody detection in whole blood.

To perform this method, 1 μm streptavidin magnetic beads (from Pierce) were prepared by gathering 100 μL 10 mg/mL beads to the side of a tube using an external magnet, aspirating out their buffer, and rinsing the beads with 200 μL of wash buffer, comprising 25 mM Tris and 150 mM NaCl (both from Sigma-Aldrich). The beads were gathered to the side of the tube using an external magnet, and the wash buffer was removed. 50 μL 200 μg/mL SARS-COV-2 biotinylated spike RBD protein (ProSci) was added to the 1 mg washed beads and left to incubate for 1 hour at room temperature at or between 20° C. and 25° C. Following this incubation, the beads were washed three times by magnetically gathering them to the side of the tube, aspirating out the supernatant, and washing them with 200 μL of wash buffer. On the final rinse, 25 μL of 0.1 M phosphate buffer was added to the mass of beads, resulting in 40 mg/mL magnetic beads coated in SARS-COV-2 spike RBD protein.

To prepare the test assembly cartridge, cartridges with channels (3×3×47 mm³) were 3D printed with Prusament UV sensitive resin from Prusa Research. Once cured, a coverslip (Fisher Scientific) was glued to the open face of the cartridge, covering the channel, and left to dry overnight at room temperature. To ensure a hydrophobic surface, 423 μL of glass water repellent (Rain-X®) was incubated in the cartridge for 30 minutes at room temperature. Following incubation, excess glass water repellent was removed and the cartridges were washed three times with water.

To prepare the aliphatic partitions, eicosane (Tm=42° C.), a higher order alkane, was used to form the TRAPs to separate each region or sub-region. First, the cartridge channels were filled with 50 μL solution containing 5 μM Amplex Red (Biotium) and 1 mM hydrogen peroxide (Fisher Scientific). To prepare the eicosane (Alfa Aesar), it was first melted by placing it in a glass vial on a hot plate at 120° C. The 30 μL of melted eicosane quickly hardened as it was deposited atop the Amplex Red/hydrogen peroxide layer. Then 60 μL 0.1 M phosphate buffer was added, followed by another 30 μL of melted eicosane, followed by 60 μL 0.1 M phosphate buffer, followed by 30 μL of melted eicosane, followed by 50 μL 100 ng/mL horseradish peroxidase (HRP)-conjugated anti-rabbit IgG antibodies (ThermoFisher), followed by 30 μL of melted eicosane. Finally, in the top zone, 2.5 μL 40 mg/mL magnetic beads coated in SARS-COV-2 spike RBD protein was added.

To prepare the sample, whole blood was withdrawn from an exposed vessel at the elbow pocket of swine forelimbs within 15 minutes after the animal was euthanized. The blood was well mixed with an EDTA-coated collection tube and subsequently stored at 4° C. Immediately preceding the experiment, the blood was spiked with varying concentrations (0-1000 ng/mL) of SARS-COV-2 spike RBD protein antibodies (ThermoFisher). 50 μL spiked blood samples were added to the top zone of the TRAP assay.

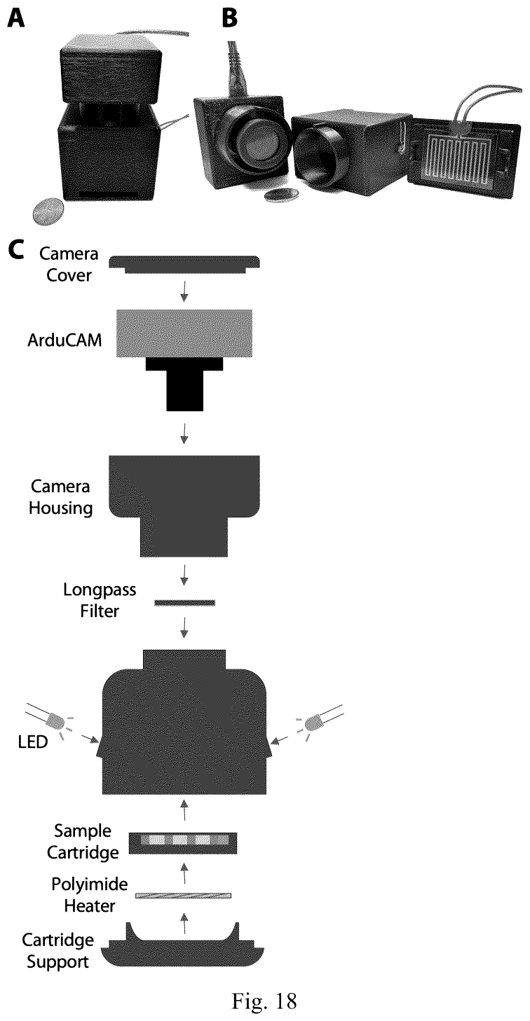

Fluorescence measurements were taken by placing a cartridge into a portable fluorescence reader as depicted in FIG. 18. The fluorescence reader is made up of 3D printed parts that secure in place an ArduCAM MT9M001 Camera with an ArduCAM USB2 Camera Shield (ArduCAM), a 570 nm longpass filter (Thorlabs) on the lens of the camera, two 525 nm LEDs to excite the sample, and a polyimide heater, shown assembled in FIG. 18A. As shown in FIG. 18B, the device opens to separate the heater from the ArduCAM to insert an assay cartridge. The ArduCAM is housed behind a longpass filter to detect fluorescence produced from a sample, which is excited by LEDs built into the device above the sample cartridge, as shown in FIG. 18C. The cartridge is placed on top of a polyimide heater to liquefy the TRAPs. The support in which the sample cartridge sits positions the sample in a precise location relative to the camera.

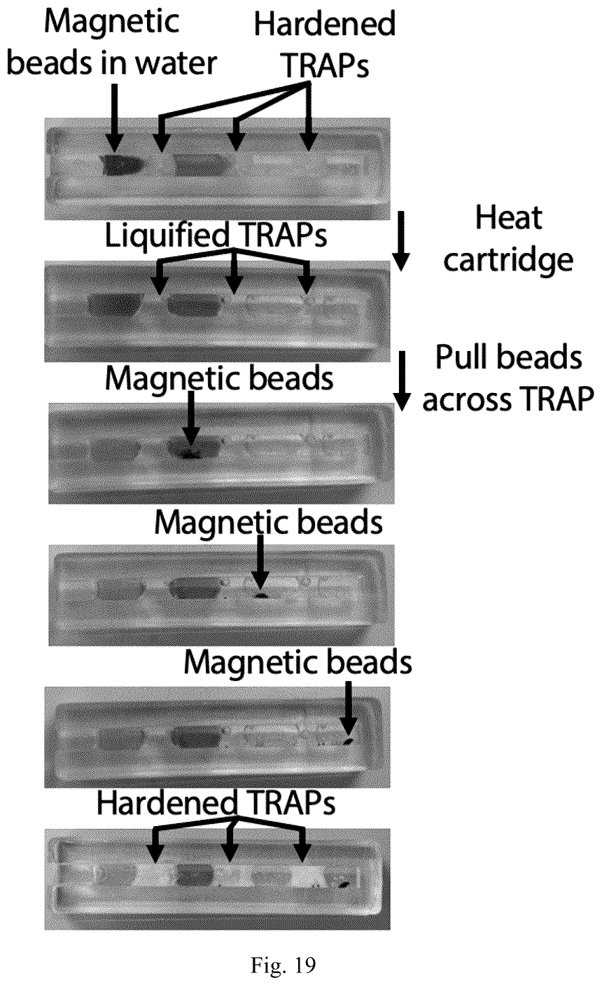

After the blood sample is added to the top zone of the assay, the cartridge was placed on the portable heater to melt the eicosane layers. The heat required to melt eicosane in each device was supplied via a polyimide heating pad that adhered to the 3D printed support that holds the sample cartridge in place (as seen in FIG. 18C). Upon placing a sample cartridge into its support, 6V of DC power was supplied to the polyimide heater. About two minutes after switching the power on, the temperature of the heater reaches eicosane's melting point. Once melted, an external magnet was used to apply a magnetic field the beads and move the beads across the first layer of eicosane, into the region containing HRP-conjugated antibodies. The external magnet was pulled at a rate of 0.2 mm/s to 10 mm/s. The heater was turned off and the beads were left to incubate for 30 minutes. After incubating, the heater was turned back on to melt the eicosane layers. Then, an external magnet was used to apply a magnetic field the beads and move the beads across the second layer of eicosane, into one of the regions containing phosphate buffer (i.e., a rinse sub-region). The beads were subsequently pulled across the third layer of eicosane, into another region containing phosphate buffer. Finally, the beads were pulled across the fourth layer of eicosane, into the detecting region containing Amplex Red and hydrogen peroxide. The heater was turned off and the fluorescence was measured by the fluorescence reader 10 minutes after the beads reach the detecting region. FIG. 19 shows photos of magnetic beads moved through TRAPs in the cartridge.

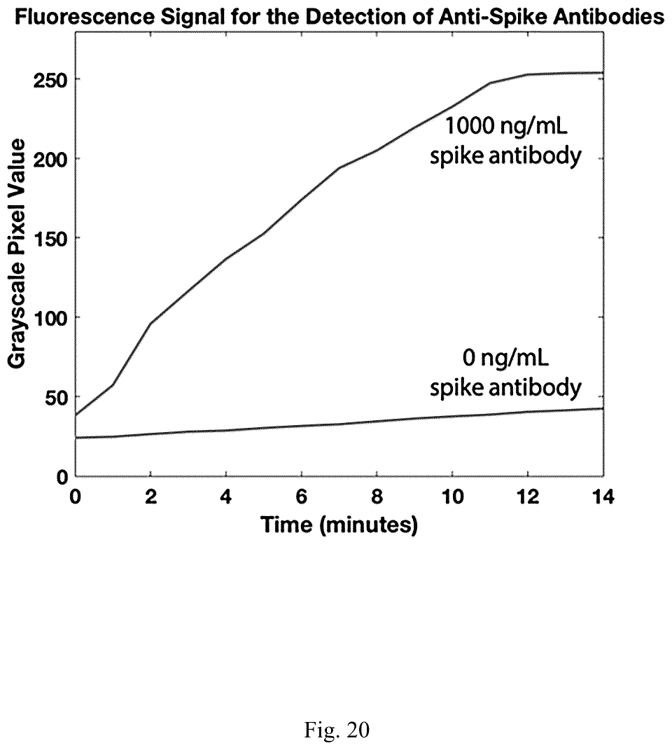

The results of the fluorescence quantification are shown in FIG. 20. FIG. 20 displays the fluorescence quantification of antibodies against SARS-COV-2 spike proteins over a 14-minute period, where the heater was turned off at t=0 minutes.

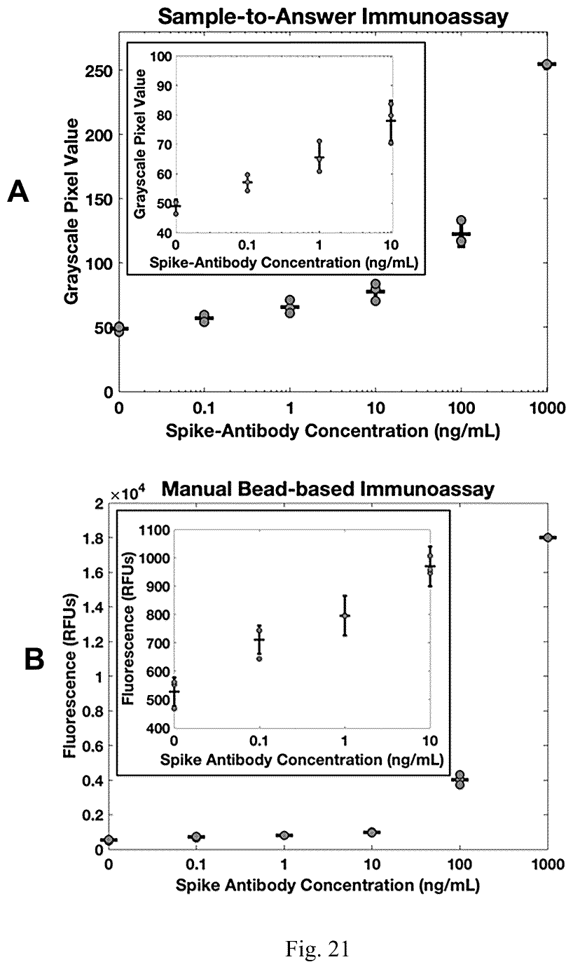

Using the International Union for Pure and Applied Chemistry (IUPAC) definition of the limit of detection (the concentration that generates a signal with a mean that is separated from the mean of the blank by three standard deviations of the blank), the limit of detection for this sample-to-answer assay was 84 μg/mL, as shown in FIG. 21A. The error bars in this figure are +1 standard deviation. Similarly, using the International Organization for Standardization (ISO) definition for the limit of detection (5% error on the blank and 5% error on a positive sample with the lowest concentration that is identifiable as positive), the limit of detection was 102 μg/mL. All values for detection limit were lower than the reported range of anti-spike antibodies in whole blood.

To compare data points, a manual bead-based ELISA assay with manual wash steps was performed by adding 2.5 μL 40 mg/mL magnetic beads coated in SARS-COV-2 spike RBD protein to 50 μL whole blood samples spiked with SARS-COV-2 spike RBD protein antibodies (0-1000 ng/mL). The beads and antibodies were left to incubate at room temperature for 30 minutes. The beads were gathered to the side of the tube using an external magnet, the supernatant was aspirated out, and they were rinsed with 200 μL of wash buffer three times. On the final rinse, the beads were re-suspended in 50 μL 100 ng/mL HRP-conjugated anti-rabbit IgG antibodies and left to incubate at room temperature for 30 minutes. The beads were gathered to the side of the tube using an external magnet, the supernatant was aspirated out, and they were rinsed with 200 μL of wash buffer three times. On the final rinse, the beads were re-suspended for 5 minutes in 5 μL elution buffer, which was comprised of 0.1 M glycine (Sigma-Aldrich) at pH 2. 5 μL of supernatant was collected and added to a 50 μL solution containing 5 μM Amplex Red and 1 mM hydrogen peroxide. Fluorescence resulting from manual bead washing was measured using a Synergy LX plate reader from BioTek (530 nm excitation, 590 nm emission). After the eluted sample was added to the Amplex Red/hydrogen peroxide solution, the liquid was moved into a 96-well plate. A fluorescence measurement was taken after 10 minutes.

Using the IUPAC definition, the limit of detection of this manual bead-based assay is 68 μg/mL, comparable to the example sample-to-answer assay (84 pg/mL). Likewise, using the ISO definition, the limit of detection of the manual bead-based assay is 80 μg/mL, similar to the performance of the example sample-to-answer assay (102 pg/mL). Both methods resulted in comparable limits of detection, suggesting the example sample-to-answer assay does not sacrifice sensitivity as it takes on key elements of point-of-care diagnostics.

FIG. 21B displays the fluorescence quantification of antibodies against SARS-CoV-2 spike proteins in whole blood using a bead-based immunoassay with a manual rinse steps and a benchtop plate reader (N=3). FIG. 16B displays the detection limit as 68 μg/mL using the IUPAC definition. The error bars are +1 standard deviation.

To be truly point-of-care, assays need to be sample-to-answer, implying that whole blood samples must be collected and loaded into the cartridge without any precise manual sample transfers. Thus, point-of-care tests cannot rely on venous blood draws performed by phlebotomists and should instead enable the patient to draw their own sample or enable easy collection at a collection site via nurse, lab technician, or physician's assistant. The device used in this example was able to pull a precise volume of whole blood directly from finger prick into the cartridge via a capillary tube. The capillary tube was built into the cap (FIG. 5A), which when contacted with the blood sample (FIG. 5B), quickly wicked up blood (FIG. 5C) until a precise volume was reached (FIG. 5D). The channel was then tapped to empty the blood into the cartridge (FIG. 5E) and the capillary tube cap was exchanged with a sealed cap after the sample was loaded (FIG. 5F). This entire process was completed in 165 seconds. The integration of this sample collection step was crucial for sample-to-answer diagnostics as there are no manual sample preparation steps required of the user.

This example demonstrated a sample-to-answer assay for the detection of anti-spike antibodies in whole blood. Also described was a detection limit below the clinical threshold cutoff to be considered positive for antibodies against the SARS-COV-2 spike protein. Fluorescence measurements were taken using a portable reader containing a built-in heater to integrate sample preparation steps into the overall system. Finally, this example has also integrated the blood collection step using a built-in capillary tube.

### Example 4

The following example provides examples of methods and devices of the present disclosure.

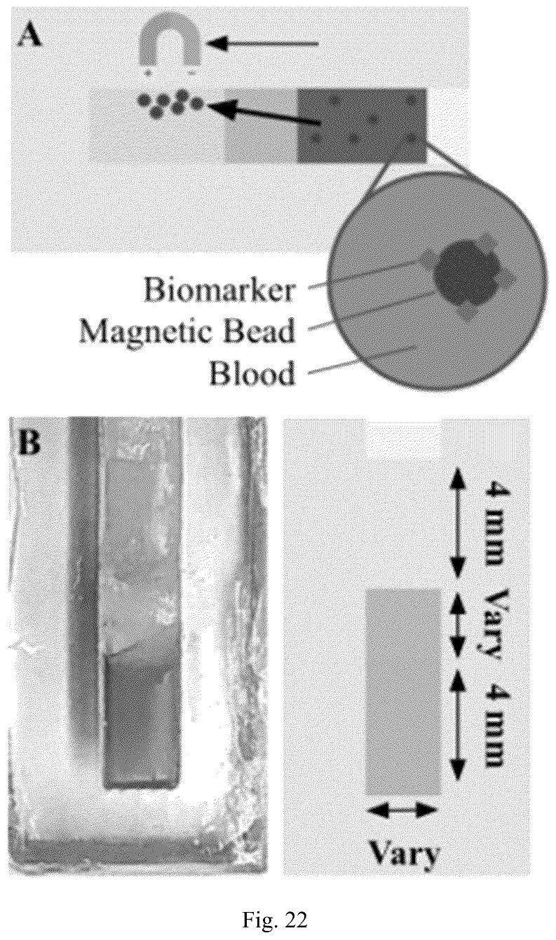

To determine the behavior of alkane in a small channel, experiments were conducted in 3D-printed channels. A range of channel sizes both hydrophobic (native resin) and hydrophilic (resin modified with fetal bovine serum) with square cross-sections were filled such that a layer of eicosane wax separated two 4 mm layers of dyed water as seen in FIG. 22. FIG. 22A illustrates a schematic application of the TRAP in which magnetic beads pull biomarkers out of a blood layer through melted alkane into a rinse zone. FIG. 22B shows a photo and schematic of a resin channel showing two dyed water layers separated by a TRAP. The channel was placed on a 60° C. hot plate either vertically or horizontally. A thinner layer of wax would break, which caused the two dyes to mix, while a thicker layer of wax kept the two dyes separated. The thickness of the wax layer (plug length) was varied to experimentally find the breakage threshold for several different sizes of channels. The geometric model equations are displayed in Example 1.

In the second experiment, magnetic beads were introduced to the system. In a horizontal 3×3 mm channel with a 2 mm thick wax layer, magnetic beads and FAM fluorophores were added to the layer on top of the wax. After melting the wax on a 60° C. hot plate, a magnet was used to move the beads across the TRAP. To verify that no leakage occurred during transfer, fluorescence measurements were taken before and after the transfer by a portable fluorescence reader.

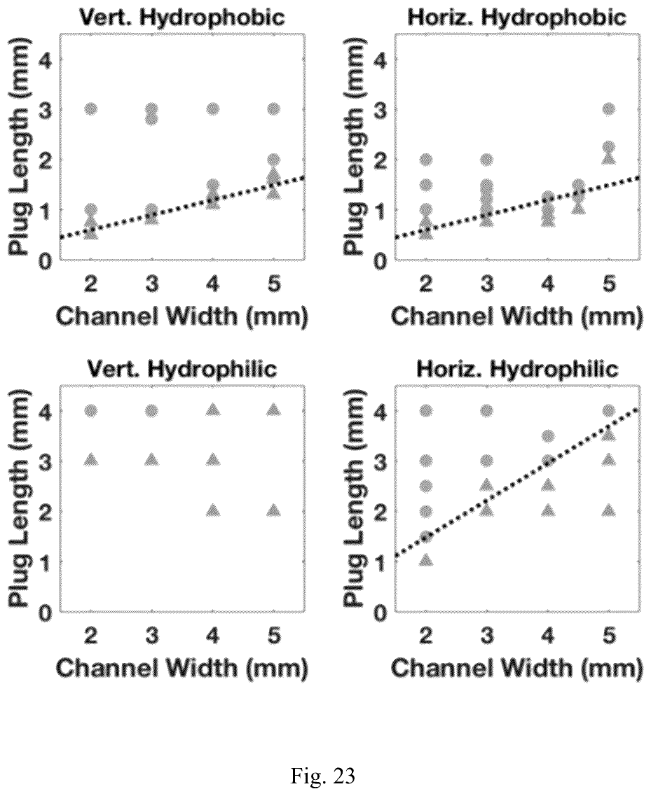

Because of the buoyancy of the alkane, TRAPs in vertical and horizontal configurations were investigated. FIG. 23 depicts the results of several combinations of plug length and channel width for vertical or horizontal configurations and hydrophobic or hydrophilic channels, where green circles indicate that the TRAP stayed, and red triangles indicate that the TRAP broke and dyes mixed. In the vertical case, it was determined that as the alkane liquefies, it is drawn to the walls of the hydrophobic resin channel, pinching in the center. Based on the plug length, channel width, and contact angle of the water-alkane-resin interface, a geometric model was produced (plotted as a dotted line in the top-left panel of FIG. 23) to predict if this pinching phenomenon results in a static or removable partition. Similar behavior is seen when the same channel is horizontal; however, this model broke down when the impact of gravity changed the behavior in channel widths around 5 mm (as seen in the top-right panel of FIG. 23).

To implement an assay in which mixing of reagents (i.e., TRAP breakage) is desired, a thin (1 mm) plug length could be used. However, in a hydrophilic channel, gravity dominates and a TRAP is more likely to break, allowing two solutions to mix. In applications where static partitions are desired, hydrophobic channels should be used while hydrophilic channels should be used when removable partitions are desired.

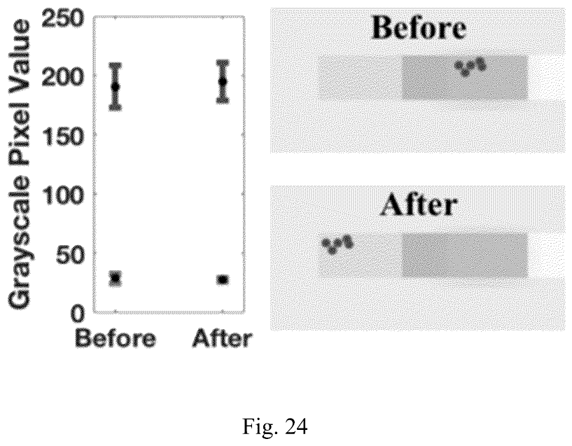

In various examples, functionalized magnetic beads are pulled through a static aliphatic partition that has been liquefied. To evaluate this diagnostic assay, the robustness against leakage after beads are moved across a TRAP was investigated. FIG. 24 shows fluorescence measurements of the water layer (red) and fluorescence layer (blue) along with their corresponding schematics of a channel before and after beads were pulled through a TRAP via a magnet. The result of this experiment showed that minimal leakage of the fluorophore through the partition occurred when transferring beads.

TRAPs can serve as low-cost automated valves that can be applied to systems where reagent manipulation is done without user interaction. The predictable behavior of leak-free partitions permeable to magnetic beads along with partitions that can be removed to allow solutions to mix at a specified time.

### Example 5

This example provides uses of systems of the present disclosure.

Described herein is a system to perform genomic detection of viruses in blood samples. The system comprises a cassette and a handheld instrument that contains a heating element, magnetic control, an LED, and a camera or optical detector. The differentiating technology is thermally responsive alkane partitions (TRAPs), which act as valves in the cassette and thus to eliminate the manual steps currently required in comparable assays.

Reagents were prepackaged in the cassette. At the bottom of the channel in the cassette are reagents for DNA/RNA amplification (a method called RT-LAMP was used, which does not require the thermal cycling that PCR utilizes). Capping these reagents in the channel is a thin alkane layer with a melt temperature of ˜60° C. (hexacosane was used). This alkane layer maintained partitioning of the amplification reagents until the alkane layer was melted. Above this alkane layer was a solution that contains a protease that digests proteins in the temperature range of 30-40° C. and that is heat-killed above 50° C. (thermolabile Proteinase K was used). This protease is used to lyse viral particles to release the genome. Above the protease layer is a thick TRAP with a melt temperature of about 40 liquefaction C (eicosane was used). Because of its thickness, this TRAP will continue to partition even after it is melted (in previous work it was demonstrated that the geometry of the TRAP dictated whether it will breach or continue to partition after melting). Above this layer was a reagent containing magnetic microbeads with aptamers attached to the surface; the aptamers bind specifically to a surface antigen on the virus (we have used an aptamer directed toward the Spike protein on SARS-COV-2). The blood sample is added to this region of the cassette to begin the assay.

In the first step, after the blood sample is added, virus binds to the aptamer on the magnetic beads. Then the cassette was heated (within the handheld instrument) to 42° C., liquefying the upper TRAP. A magnet was used to pull the beads through the TRAP into the protease layer. Then the virus is incubated with the protease, causing the genomic nucleic acids to be released. Then the cassette was heated to 65° C., killing the protease and melting the lower TRAP. Because the lower TRAP is thin, it breaches, causing the amplification reagents to mix with the viral genome (the protease has been heat killed by this time and will not destroy the polymerase). During amplification, various methods can be used to measure the creation of new DNA (for example, an intercalating dye can be used to observe the increasing fluorescence in real time during amplification). A rapid and significant increase in fluorescence indicated that the viral genome was present.

### Example 6

This example provides uses of systems of the present disclosure.

The diagnosis of bloodborne viral infections (viremia) is currently relegated to central labs because of the complex procedures required to detect viruses in blood samples. The development of point-of-care diagnostics for viremia would enable patients to receive a diagnosis and begin treatment immediately instead of waiting days for results. Point-of-care systems for viremia have been limited by the challenges of integrating multiple precise steps into a fully automated (i.e., sample-to-answer), compact, low-cost system. It was recently reported the development of thermally responsive alkane partitions (TRAPs), which enable the complete automation of diagnostic assays with complex samples. Described herein is the use of TRAPs for the sample-to-answer detection of viruses in blood using a low-cost portable device and easily manufacturable cassettes. Specifically, demonstrated herein is the detection of SARS-COV-2 in spiked blood samples, and it also shows the system detects viremia in COVID-19 patient samples with good agreement to conventional RT-qPCR. It is believed the sample-to-answer system described herein can be used to rapidly diagnose SARS-COV-2 viremia at the point of care, leading to better health outcomes for patients with severe COVID-19 disease, and that this system can be applied to the diagnosis of other life-threatening bloodborne viral diseases, including Hepatitis C and HIV.

Described herein is a magnetofluidic approach for S2A from complex samples that does not require pumps or microfluidics, and that is stable to device agitation. This approach is enabled by Thermally Responsive Alkane Partitions (TRAPs), which we described and characterized in previous work. A TRAP is solid at ambient temperature and liquid at a moderately elevated temperature, specific to the type of alkane. When melted in between two partitioned liquids in a millimeter-scale channel, if thin enough the TRAP will breach and the two liquids on either side will combine (i.e., a removable TRAP). This behavior enables automatic controlled reagent combination. At the same time, if the TRAP is sufficiently thick, the melted TRAP will continue to partition the two liquids (i.e., a stationary TRAP). In this state, the TRAP becomes permeable to magnetic microbeads but continues to partition aqueous reagents. It was proven that magnetically transferring beads (˜1 μm diameter) across a TRAP caused minimal leakage that can be further negated with the addition of rinse layers.

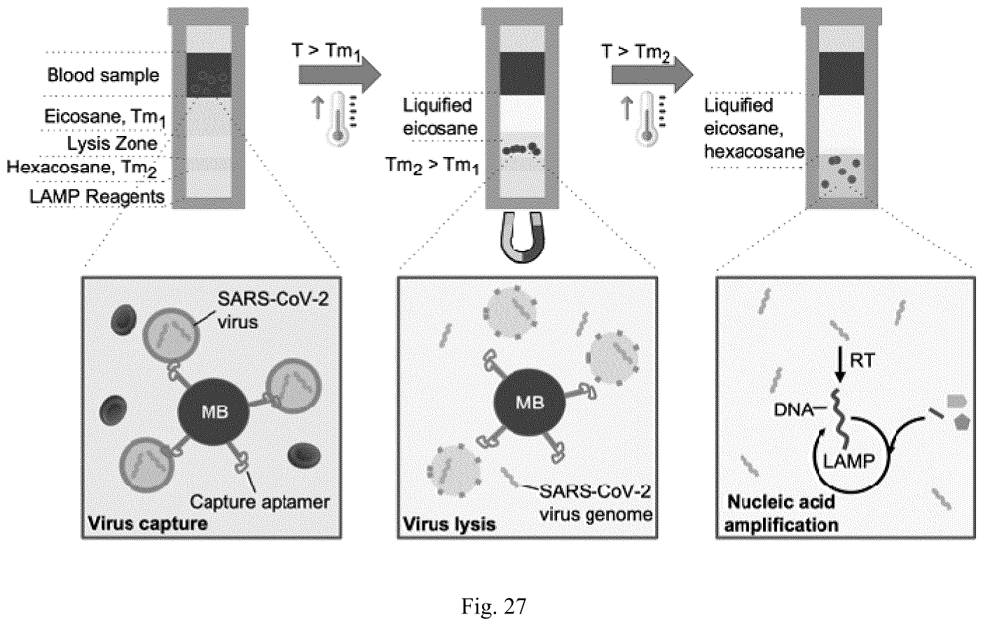

Removable TRAPs were previously applied to detect circulating histones in blood, and we applied stationary TRAPs to create a portable PoC system to detect SARS-CoV-2 antibodies from whole blood. The current work refines the TRAP system to be more robust, more user-friendly, and more capable. Specifically, demonstrated herein for the first time S2A detection of SARS-COV-2 viremia in a low-cost portable format. To do this, both TRAP behaviors described above (removable and stationary), as illustrated in FIG. 27, were combined. Virus isolation from a whole blood sample is achieved by capturing virus onto aptamer-functionalized paramagnetic microbeads and magnetically pulling the beads through a melted stationary TRAP. Effective virus particle lysis was shown via an incubation with Proteinase K. Automatic initiation of reverse transcription loop-mediated isothermal amplification (RT-LAMP) is accomplished by combining reagents upon melting a removable TRAP following lysis. Finally, we demonstrate the PoC system's ability to distinguish SARS-COV-2-positive and SARS-COV-2-negative spiked whole blood samples as well as patient blood plasma samples collected during the height of the COVID-19 pandemic was demonstrated.

Materials. Cassettes were fabricated by cutting an acrylic sheet with 24 mm thickness (McMaster-Carr) into 6.35 mm diameter cylinders each with a channel 3.2 mm in diameter and 23 mm deep via CNC. The alkanes in this work include n-eicosane (melting point ˜37° C.), 99%, and n-hexacosane (melting point ˜57° C.), 99%, both from ThermoFisher Scientific. Pierce™ Streptavidin Magnetic Beads (1.05 μm diameter) were purchased from ThermoFisher Scientific. Antarctic Thermolabile Uracil DNA Glycosylase (UDG) was purchased from NEB and is used throughout this work paired with DNA amplification reactions that result in nucleic acid products containing uracil bases. UDG helps reduce the risk of carry-over contamination from previous DNA amplification reactions. WarmStart® Multi-Purpose LAMP/RT-LAMP Master Mix with UDG was purchased from NEB.

Thermolabile Proteinase K was also purchased from NEB. Mineral oil (light, lab grade) was purchased from VWR. Gamma-irradiated SARS-COV-2 was supplied as a ˜10⁹ copies/mL stock solution from BEI Resources. The biotinylated SARS-COV-2 RBD aptamer used in this work as well as LAMP primers for the N15 gene were purchased from IDT and their sequences are described below.

RT-LAMP Reaction. Based in part on the recommended protocol from the NEB website, benchtop LAMP tests comprises the following procedure. A solution containing 1× WarmStart® Multi-Purpose LAMP/RT-LAMP Master Mix with UDG (NEB), 1×LAMP Fluorescent Dye (NEB), and LAMP primers (1.6 μM FIP/BIP, 0.2 μM F3/B3, and 0.4 μM LoopF/B) for the N15 gene is created using nuclease-free water to reach the appropriate concentrations. The volume made equals 20 μL times the number of samples being tested plus one to account for possible pipetting error. After vortexing the solution, 20 μL is loaded into PCR tubes. Then 5 μL of each sample is added to each tube. Finally, each tube is inserted into a MiniOpticon (Bio Rad) which heats the solutions to 65° C. for 60 minutes while recording the fluorescence of each solution (one reading every 30 seconds).

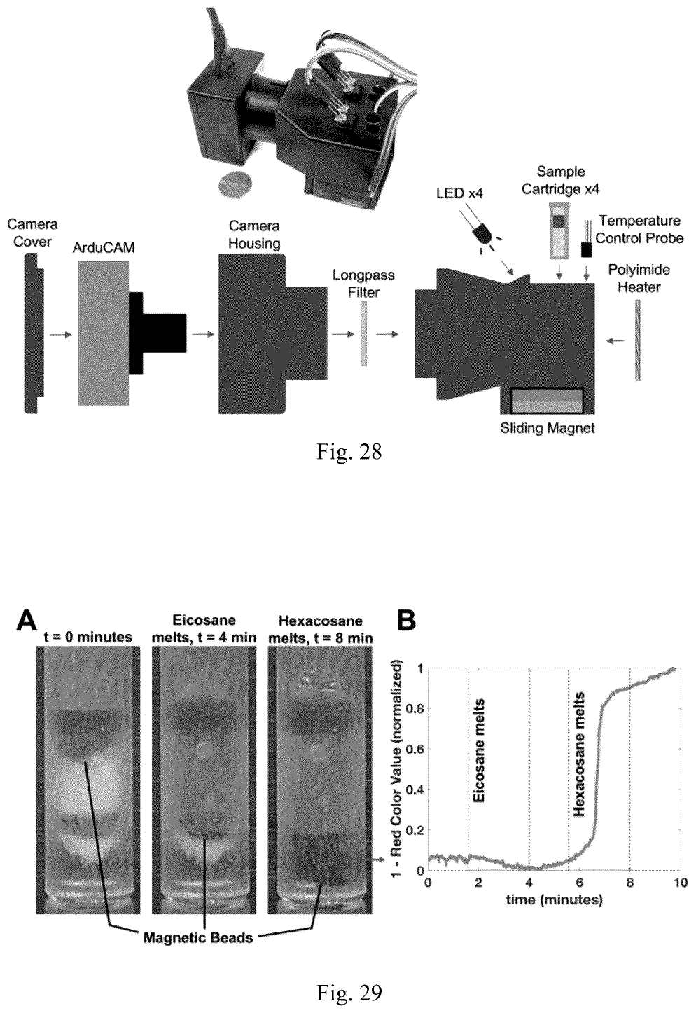

Portable Controller and Reader Device. The device used in this Example consists of an ArduCAM MT9M001 Camera with an ArduCAM USB2 Camera Shield, a 530 nm longpass filter (Thorlabs), four 470 nm blue LEDs (one for each sample cassette), a DS18B20 digital temperature sensor, a pair of 30 mm×40 mm polyimide heaters (DWEII) positioned about 8 mm from the inserted cassettes, and two 10 mm tall neodymium magnet cylinders 25 mm in diameter (K & J Magnetics) initially positioned below the inserted cassettes. Components are housed in a 3D printed casing that fits in the palm of a hand. A photo of the device and an exploded view are shown in FIG. 28.

TRAP Demonstration. To show the capabilities of the two-TRAP assay displayed in FIG. 27, the following experiment was conducted. Aliquots of eicosane and hexacosane were melted on a hot plate set to 180° C. 15 μL of a yellow dyed solution containing 0.1% Tween 20 in water (a similar surfactant concentration to that of the LAMP master mix used in this work) was loaded into the bottom of a channel. 8 μL of hexacosane was loaded on top of the yellow solution to seal it in place. 10 μL of blue dyed water was loaded on top of the hardened hexacosane. 35 μL of eicosane was placed on top of the blue solution to seal it in place. Finally, 25 L of a red dyed solution containing 100 μg of streptavidin magnetic beads was loaded on top of the hardened eicosane. The assembled cassette was then placed onto a magnet with a polyimide heater attached. The heater increased the temperature of the magnet and cassette to about 65° C. while a video of the resulting behavior was recorded. To quantify mixing, the red color value of the bottom layer of the cassette was extracted.

Virus Particle Lysis. In the proposed assay, which captures and isolates virus particles from complex samples, it is necessary to lyse the virus particles to release the virus genome for amplification and detection. In this Example, thermolabile Proteinase K (NEB) was used to lyse virus particles. To determine how effective Proteinase K is at lysing virus particles, first a pure (free of extra-viral RNA) virus particle solution must be made. This was done by incubating a 50 μL solution including PBS and 10000 copies/μL gamma-irradiated SARS-COV-2 (BEI Resources) with 1 μL (20 μg) Monarch RNase A (NEB) for 5 minutes at 56° C. 1.6 μL (64 units) of RNase Inhibitor (Murine, NEB) was added to the solution after the incubation. Six of these extra-viral RNA-free SARS-COV-2 solutions were made. In three of them, 2.5 μL (0.3 units) thermolabile Proteinase K was added. These three solutions incubated at 37° C. for 15 minutes to allow Proteinase K to digest proteins. Then the solutions incubated at 55° C. for 10 minutes to inactivate the Proteinase K. To prepare the RT-LAMP detection reaction, nine 20 μL solutions were created as described above in the RT-LAMP Reaction subsection. 5 μL of each solution that underwent Proteinase K incubations as well as each solution that did not was added to LAMP reagents to create six separate LAMP reactions. Three more reactions were prepared with 5 μL of PBS used as a negative control. Each solution then incubated at 65° C. for 60 minutes, with fluorescence measurements taken every 30 seconds in the MiniOpticon.

Virus Viability Across a Melted TRAP. In order for the proposed assay to work properly, captured virus particles must remain intact and attached to magnetic beads while subjected to the increased temperature of a melted TRAP. To determine whether viruses remain viable after crossing a TRAP, the following experiment was conducted. First, 900 μg of streptavidin magnetic microbeads were rinsed with wash buffer then incubated with 225 fmol biotinylated capture aptamers in 18 μL of PBS for 60 minutes. Following the incubation, 12 μL of the mixture was added to a 178 μL solution containing PBS and 10000 copies/μL gamma-irradiated SARS-COV-2. Simultaneously, the remaining 6 μL of the aptamer-bead mixture was added to 84 μL PBS (to act as the no-target control). Both mixtures incubated for 15 minutes. Eicosane was melted on a hot plate set to 180° C. Six cassettes were loaded initially with 30 μL of the aptamer-bead-virus mixture, while three cassettes were loaded with 30 μL of the no-target control mixture. 40 μL of melted eicosane was added to the three cassettes loaded with the no-target control and to three of the cassettes that were loaded with the aptamer-bead-virus mixture to form TRAPs. To the three cassettes that remained, mineral oil was used in place of eicosane. 50 μL of water was then added to each cassette, with a careful deposition on top of the oil to ensure no mixing occurred between the layers. The six cassettes containing TRAPs were placed horizontally on a hot plate set to 65° C. for 5 minutes to melt the TRAPs. Once melted, a neodymium magnet was used to transfer the beads across each melted TRAP, as well as across the oil partition in the cassettes (not on the hot plate). After bead transfer, the cassettes with TRAPs were removed from the hot plate to allow eicosane to harden. Once the eicosane hardened, 40 μL of the mixture on top of each TRAP and oil partition (now containing the magnetic beads) was pipetted out and into separate tubes each containing 1 μL of thermolabile Proteinase K. The nine tubes incubated at 37° C. for 15 minutes to allow Proteinase K to break down proteins in any virus particle that made it across the TRAP or oil partition. Then a 10-minute incubation at 55° C. inactivated the Proteinase K. An RT-LAMP detection reaction was prepared in the meantime: nine 20 μL solutions were created as described above in the RT-LAMP Reaction subsection. 5 μL of the supernatant of each mixture that underwent Proteinase K incubations (obtained by moving the beads out of the way of a pipette via a magnet) was transferred to each 20 μL LAMP solution. Each solution was then incubated at 65° C. for 60 minutes while fluorescence data was recorded in the MiniOpticon.

Sample-to-Answer SARS-COV-2 Blood Test. To demonstrate that the S2A system can detect viruses in blood, the following experiment was conducted. 100 fmol biotinylated capture aptamers were attached to 400 μg streptavidin magnetic beads via a 15-minute incubation in 8 μL of PBS. A LAMP solution and a lysis solution were prepared by combining and mixing the following reagents. 1) LAMP solution: 50 μL Warm Start® Multi-Purpose LAMP/RT-LAMP 2× Master Mix with UDG (NEB), 2 μL 50×LAMP Fluorescent Dye (NEB), and 8 μL water. 2) Lysis solution: 4 μL (˜0.5 units) Thermolabile Proteinase K, 10 μL LAMP primer mix containing 16 μM FIP/BIP, 2 μM F3/B3, and 4 μM LoopF/B for the N15 gene, 2 μL (2 units) Antarctic Thermolabile UDG, and 24 μL water. 15 μL of the LAMP solution was added to the bottom of four acrylic cassettes, followed by an 8 μL hexacosane seal, then 10 μL of the lysis solution was added after the hexacosane hardened, then 38 μL eicosane, then 2 μL of the bead mixture after the eicosane hardened. 20 μL of whole blood (obtained via finger prick) with 7 μg sodium polyanethole sulfonate (to prevent blood from coagulating around the magnetic beads), spiked with 5 μL of varying amounts of gamma-irradiated SARS-COV-2 in PBS was added to each cassette. The cassettes were capped, then inserted into the portable fluorescence reader to commence the assay reactions.

The cassettes are subjected to 45° C. for 6 minutes to melt the first TRAP (made of eicosane); after it melts, the beads are pulled through the eicosane by a magnet at the bottom of the cassettes. After 5 minutes, the magnet positioned below the cassettes is moved away to remove the beads from its influence. At this point, the magnetic bead complexes are in the lysis solution, isolated from the blood sample (FIG. 27). The heating element within the fluorescence reader is shut off for 6 minutes to allow Proteinase K to degrade any virus envelope proteins, compromising the stability of virus shells and releasing viral genome from virus particles present in the mixture. The heating element is turned on again to deactivate the Proteinase K, melt the second TRAP (made of hexacosane) to combine LAMP reagents, and initiate the LAMP reaction. The space within the device stabilizes to 65° C. after about 15 minutes. This temperature is held until 60 minutes have passed since the insertion of the cassettes. Fluorescence measurements are recorded as normalized grayscale pixel values collected by the ArduCAM. These measurements begin 23 minutes into the test as that is the point in which the hexacosane TRAP melts, initiating the LAMP reaction.

SARS-COV-2 Patient Plasma Test. Plasma samples (deidentified) from COVID-19 patients were obtained from the University of Maryland Medical Center. Samples were acquired from hospitalized COVID-19 patients between the dates 2020 Sep. 18 and 2022 Feb. 15 under approved IRB protocols. Each sample was tested by the MiniOpticon benchtop RT-qPCR system. Using the QIAamp Viral RNA Kit (Qiagen), 140 μL of each plasma sample was filtered into 60 μL of elution buffer containing extracted virus genomes that may have been present in the plasma sample. 5 μL of the resulting solution was added to a 1-step RT-qPCR master mix (Promega). Each solution underwent a 15-minute reverse transcription step at 45° C. for 15 minutes, followed by two minutes in a 95° C. initial denaturation step, then 45 PCR cycles with each cycle being 95° C. for 3 seconds, and 55° C. for 30 seconds. The threshold cycle in this Example is defined as the first PCR cycle that resulted in a fluorescence measurement higher than 0.011 AU. To estimate the number of copies in each solution, dilutions of SARS-COV-2 (BEI) were subjected to the same sample treatment and qPCR reaction, and a standard curve was created. Cycle thresholds from tested patient samples were compared to the standard curve. Plasma samples were also tested with the S2A diagnostic described herein. The experimental setup above in the Sample-to-Answer SARS-CoV-2 Blood Test subsection was repeated but instead of adding 20 μL of whole blood spiked with 5 μL of SARS-COV-2, 25 μL of plasma from COVID-19 patients was loaded into the cassettes.

TRAP Operation. Previous work has shown that by melting a removeable TRAP, when pre-partitioned reagents mix can be controlled, simply by adjusting temperature. It was shown stationary TRAPs that enable a sample-to-answer immunoassay. In this Example, which presents an assay capable of detecting virus genomes from complex samples, both forms of TRAPs are utilized. A stationary TRAP is required so that, when melted, it will prevent the sample (e.g., whole blood) from interfering with detection chemistry while still allowing virus particles captured onto magnetic beads to transition to the lysis reagent. Meanwhile, a removeable TRAP enables the sequential steps of virus lysis and RT-LAMP. The RT-LAMP reagents must be sequestered during proteolytic lysis (to avoid degradation of the polymerase) and then added following the heat-kill of the protease. Thermolabile Proteinase K is inactivated at a temperature lower than the melting temperature of the removeable TRAP, and thus when increasing the temperature to release the TRAP and combine the reagents, the Proteinase K is inactivated. Once the removeable TRAP is melted, the nucleic acid amplification reaction (RT-LAMP) commences.

FIG. 29A illustrates the process described above using dyes so that it is observable. The red solution (representing the blood sample in the assay) remains separated from the blue solution (representing the lysis reagent layer) while the beads traverse the stationary TRAP. As the temperature increases to melt the removeable TRAP, the blue solution and the yellow solution (representing RT-LAMP reagents) quickly mix. The sharp slope in FIG. 29B indicates a quick mixing upon melting the removeable TRAP. This demonstrates that the combination of the two types of TRAPs in a single cassette can maintain partitioning of the blood sample away from the assay while sequentially enabling the assay steps, including the precise addition of reagents and rapid mixing.

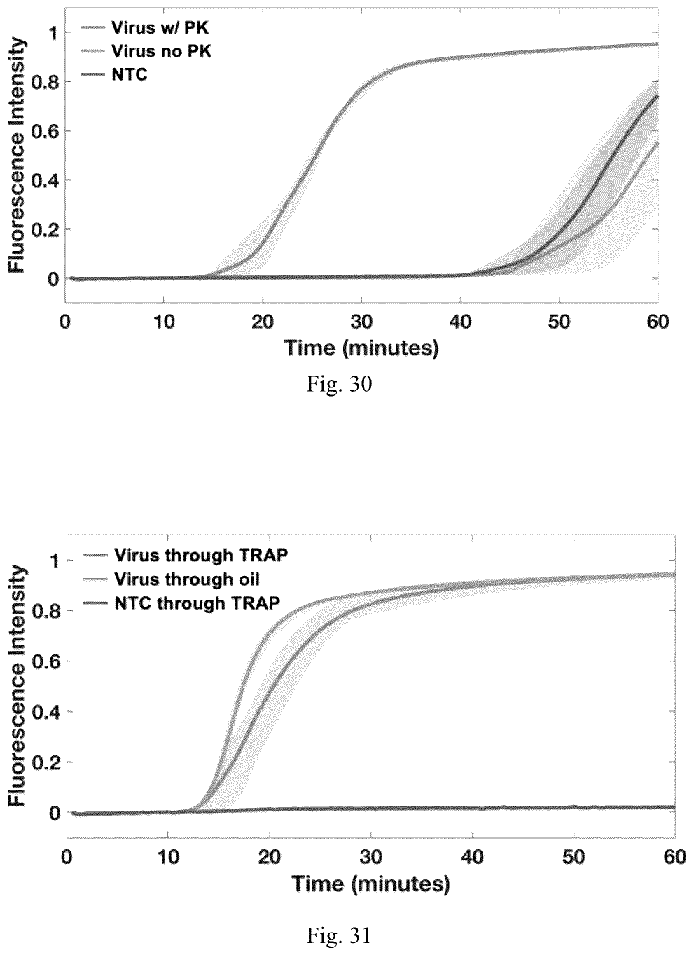

Thermolabile Proteinase K Enables RNA Release from Virus and is Compatible with Sample-to-Answer LAMP. It has been shown that Proteinase K can act as a viral lysis reagent and streamline sample preparation before RT-qPCR. It works by breaking down proteins which are abundant in the envelope of many viruses. Utilized in this Example is a thermolabile Proteinase K that is heat-inactivated below the operation temperature of LAMP. Notably, it is active at a temperature above the melt temperature of the stationary TRAP and below the temperature of the removeable TRAP. FIG. 30 demonstrates the effectiveness of Proteinase K at lysing the SARS-COV-2 virus. The data compares the LAMP-based amplification of the SARS-COV-2 genomes for three aliquots of virus treated with Proteinase K to three samples that were not treated. Note that no subsequent purification steps are performed after lysis; the sample is heated to inactivate the Proteinase K and then directly added to LAMP master mix. As FIG. 30 shows, the sample treated with Proteinase K amplified quickly, while the untreated sample is essentially not detected.

Aptamer-Beads Pull Virus Through a Melted TRAP. In previous work, it was demonstrated that antibody-antigen complexes attached onto magnetic bead traversed melted stationary TRAPs without releasing. Here, however, the cargo is virus particles which are captured via aptamers instead of antibodies. Therefore, it was necessary to investigate whether the bead-aptamer-virus complex remains viable after traversing a melted TRAP. The amplification of virus that traversed a melted eicosane TRAP on aptamer-functionalized magnetic beads with the amplification of virus that was similarly pulled through a mineral oil partition at room temperature. LAMP data from both experiments is presented in FIG. 31. The timing of the amplification signal for the two cases is not statistically significantly different. It was concluded that the elevated temperature of the TRAP results in minimal or no virus loss while beads are traversing a melted TRAP.

Sample-to-Answer Detection of SARS-COV-2 in Blood. To demonstrate the capability of the S2A system to detect viruses in whole blood, whole blood (drawn from finger pricks) was spiked with specific concentrations of SARS-COV-2 virus and tested it in our dual-TRAP cassette. Specifically, 20 μL of whole blood was spiked with 5 μL of PBS containing 5000, 500, 50, 5, or 0 copies of SARS-COV-2 (according to the stock concentration reported by BEI). FIG. 32A shows the average amplification curves for three samples with 5000 copies (orange curve) and three blank samples (blue curve). The curves show rapid amplification for the positive sample with minimal variation in the time to positive (TTP); additionally, the curves show a significant amount of time between the TTP of the positive and negative samples.

FIG. 32B reports the results of 35 different blood sample tests, seven for each concentration of spiked SARS-COV-2. If a blood sample resulted in a TTP of 22 minutes or less after the initiation of the LAMP reaction, the sample was deemed positive. The samples that had a TTP of greater than 22 minutes or never amplified for the duration of the test were deemed negative. Samples containing at least 500 copies of SARS-COV-2 resulted in 100% accuracy. One false negative was reported for the seven samples with 50 copies, and two false negatives were reported for the seven samples with 5 copies. Thus, without any sample preprocessing, highly sensitive detection of virus directly from a whole blood sample is made possible by our diagnostic system. This capability to detect virus directly from whole blood has potential to significantly improve the viremia diagnostic pathway, drastically decreasing time to diagnosis.

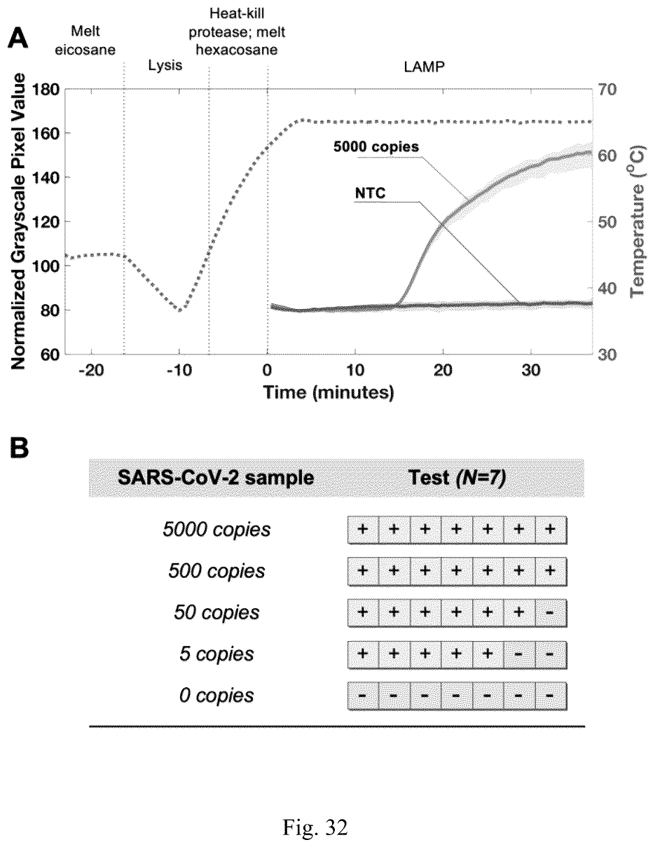

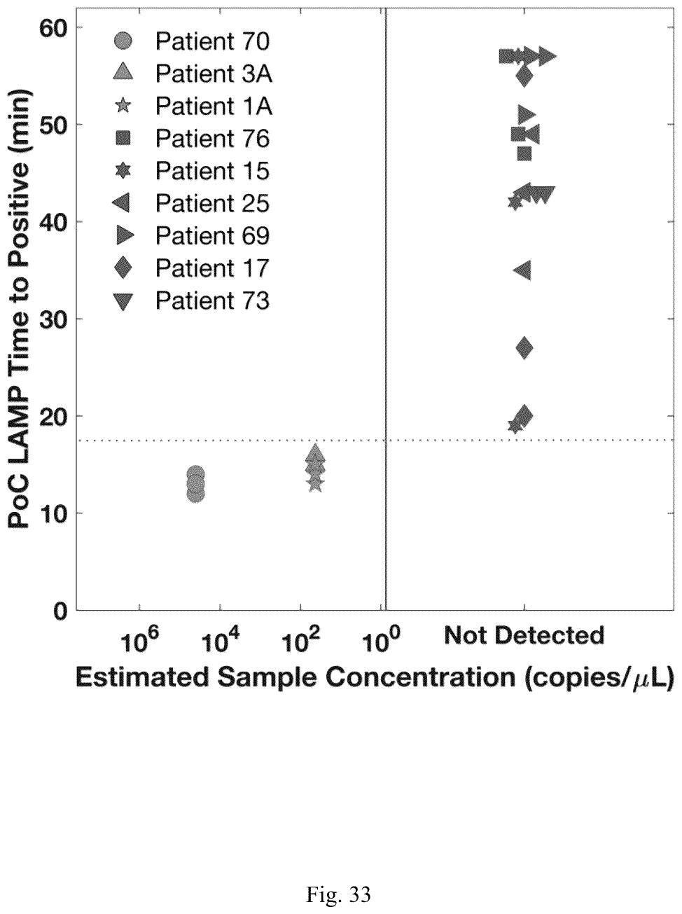

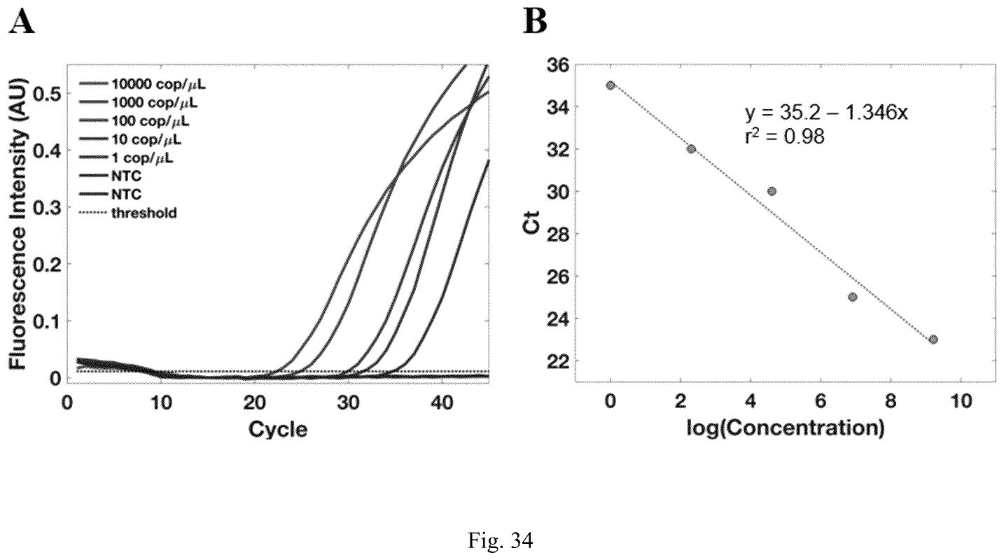

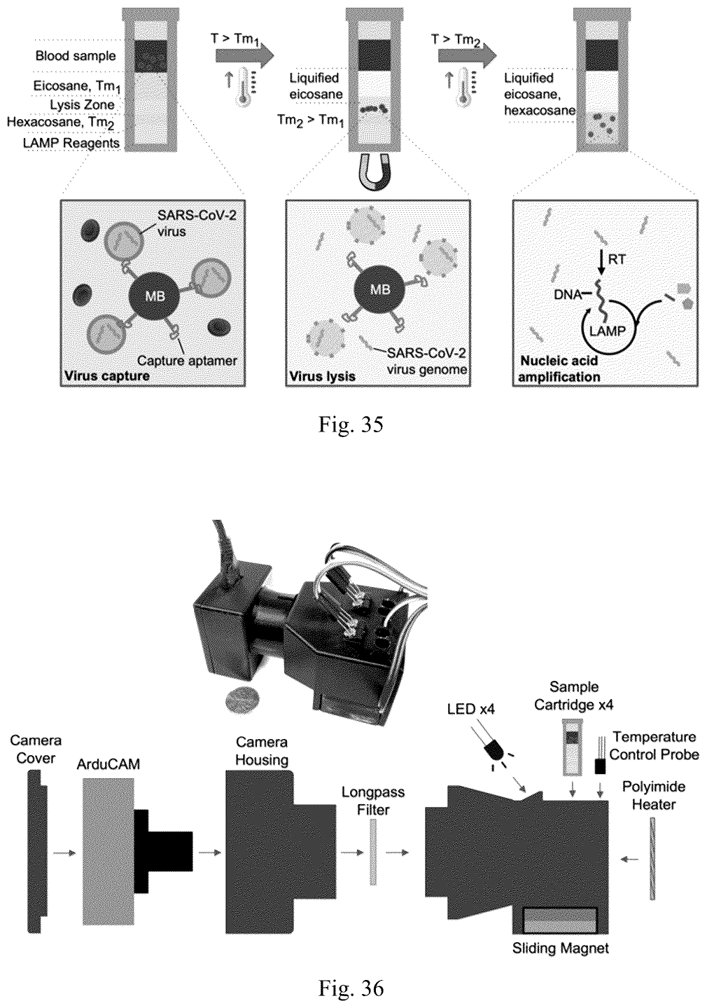

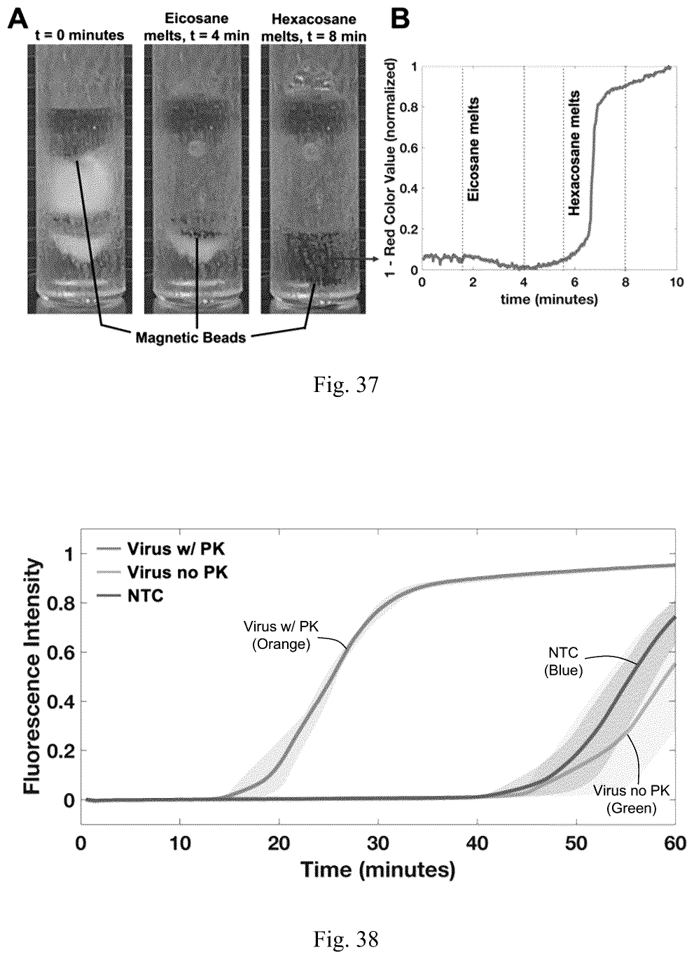

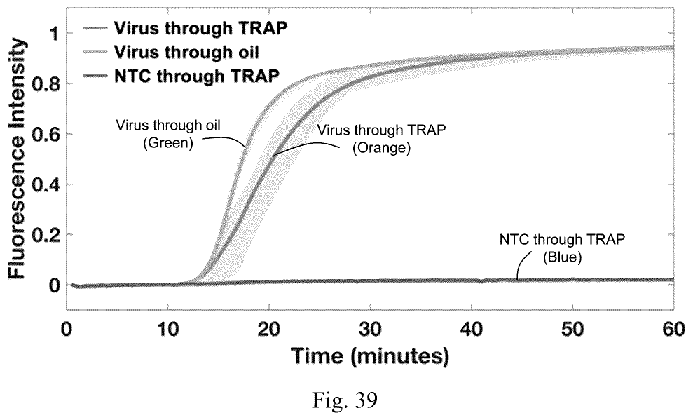

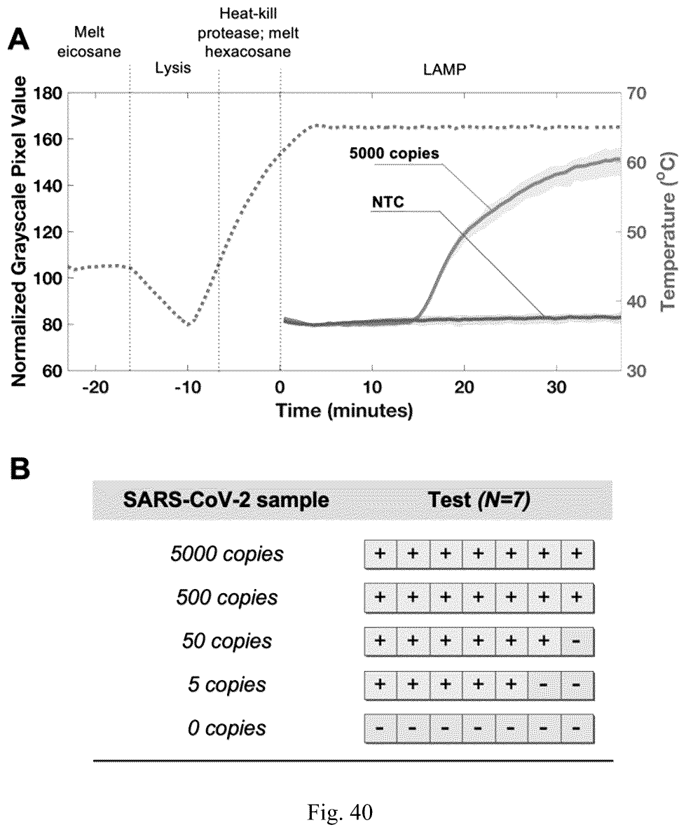

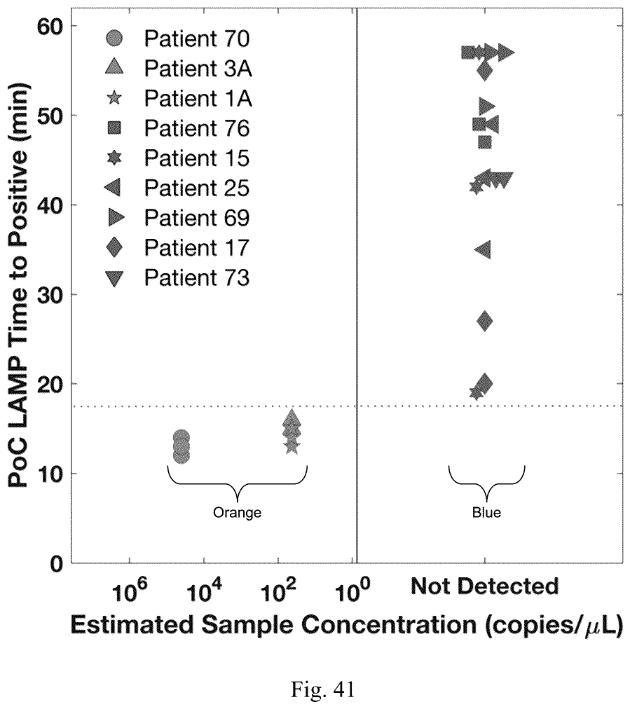

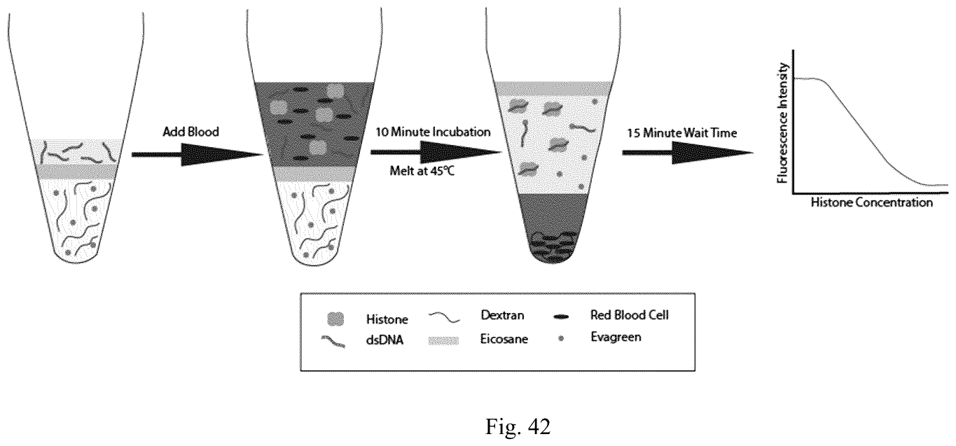

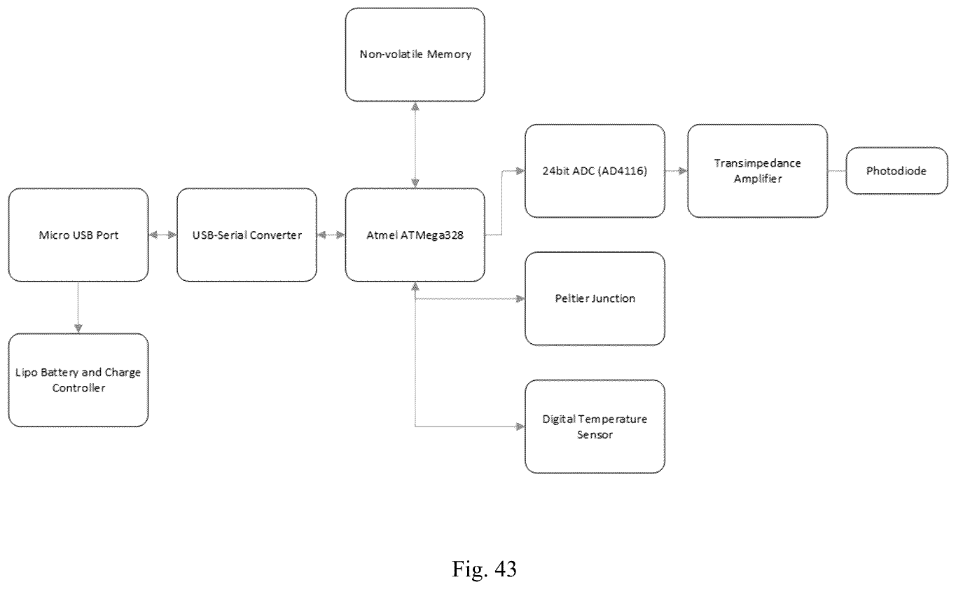

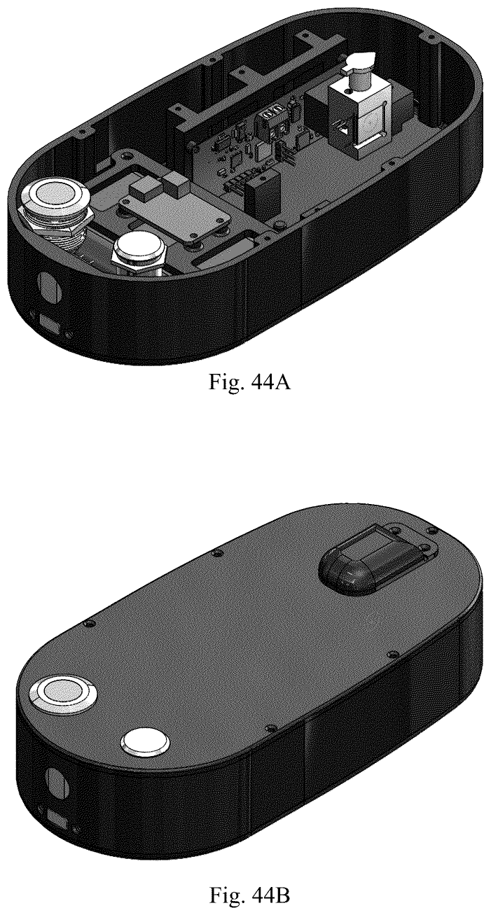

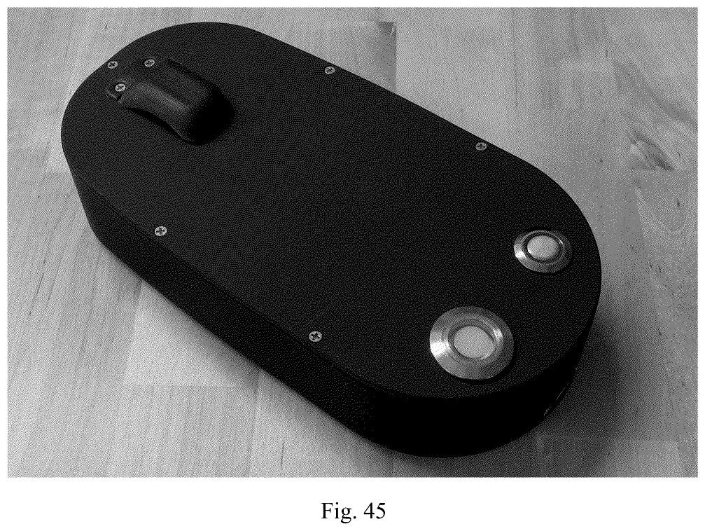

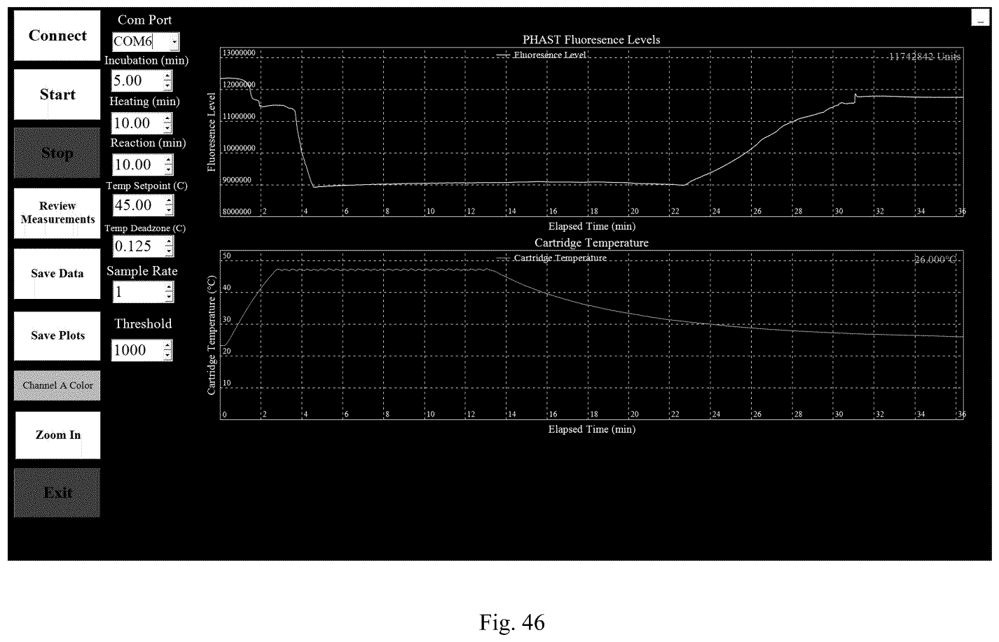

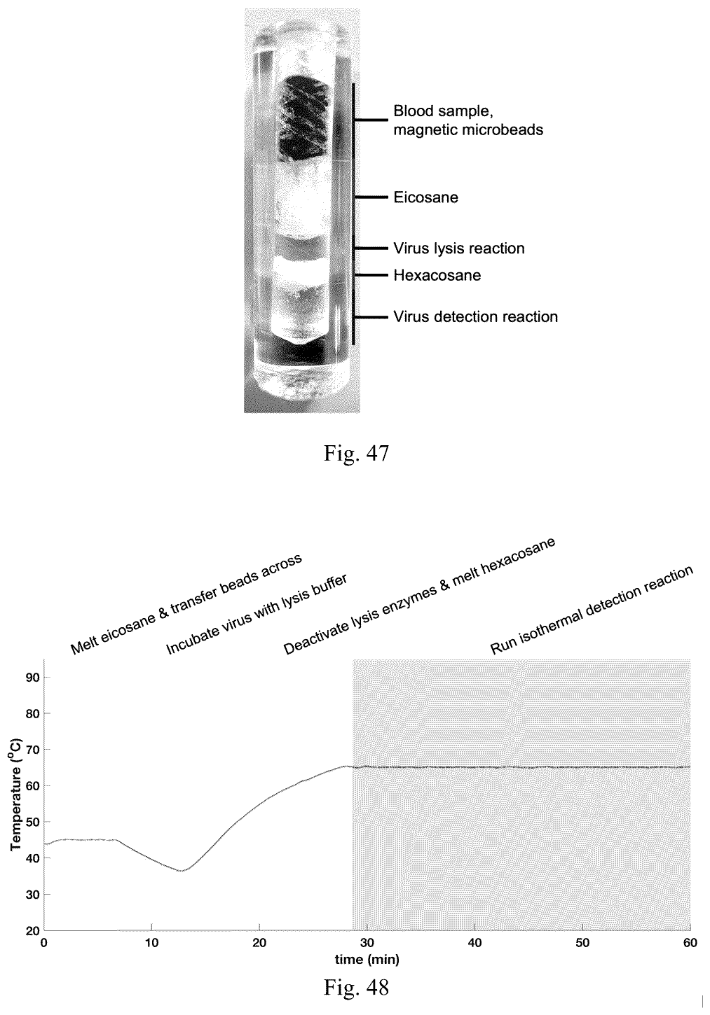

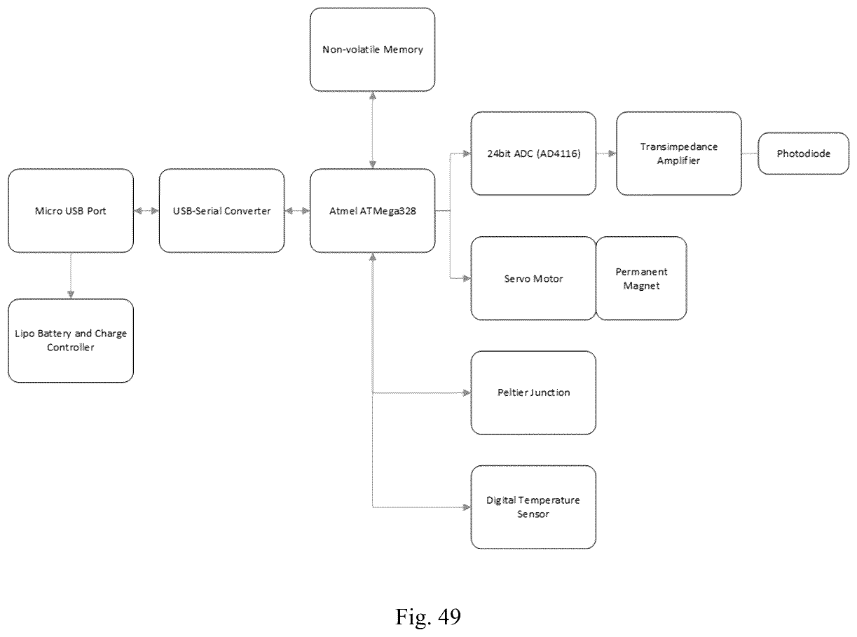

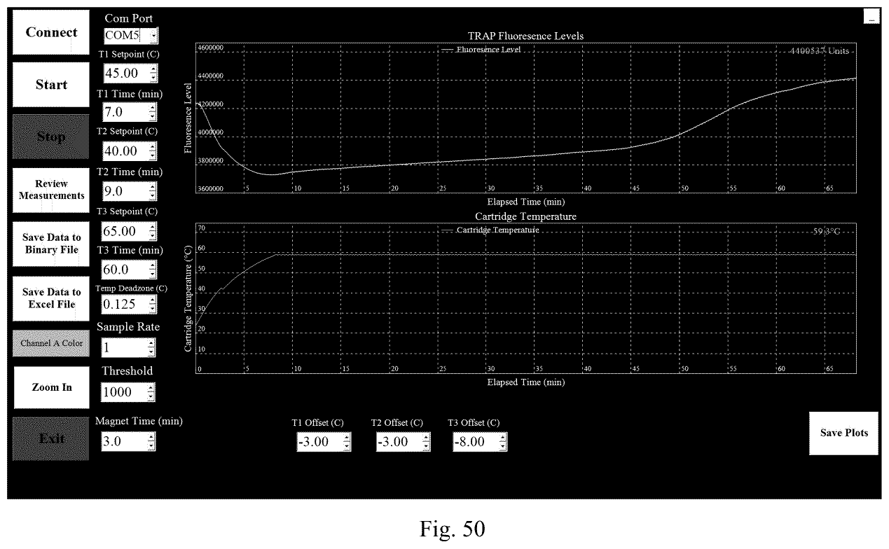

Detection of SARS-COV-2 in COVID-19 Patient Plasma Samples. To further validate the capability of our system to detect viremia, the dual-TRAP cassette and handheld device was used to test plasma samples from patients with severe COVID-19 disease. The samples were first tested by conventional RT-qPCR; RNA in the samples was quantified using RT-qPCR. Three samples had quantifiable RNA while six samples appeared to have no RNA (reported as “not detected”). These nine patient samples were then tested with our S2A system. The correlation between the S2A system and conventional RT-qPCR is presented in FIG. 33. The three patient samples that had quantifiable RNA had the lowest time-to-positive with the S2A system; each LAMP reaction had a TTP within 16 minutes of reaction initiation (i.e., when the hexacosane TRAP melted). All of the LAMP signals in our device that resulted from testing SARS-COV-2-negative (i.e., not detected) plasma samples occurred at later time points compared to the positive samples, with many not appearing until after 30 minutes into the LAMP reaction. Two trials of the negative samples, however, resulted in a LAMP signal close to the positive samples. These results suggest that our S2A system is able to distinguish between viremia-positive and viremia-negative patient samples.

In this Example, it was demonstrated that the sample-to-answer detection of SARS-COV-2 virus in blood samples. The system positively identified the virus in 100% of blood samples spiked with 500 copies/sample and greater than 70% of blood samples spiked with 5 copies/sample. In addition, the system showed good agreement with conventional RT-qPCR in detecting SARS-COV-2 viremia in banked samples from patients with severe COVID-19 disease. Notably, the S2A system utilizes an easy-to-manufacture cassette and an automated portable device that does not require microfluidics, a pump, or precise sample handling steps.

DNA Sequences
Biotinylated CoV2-RBD-4C aptamer
(SEQ ID NO: 1)
5′-ATCCAGAGTGACGCAGCATTTCATCGGGTCCAAAAGGGGCTGCTCGG
GATTGCGGATATGGACACGTTTTTTTT/3Bio/ - 3′
LAMP Primers (N15 gene)
FIP
(SEQ ID NO: 2)
5′ - TGCTCCCTTCTGCGTAGAAGCCAATGCTGCAATCGTGCTAC -
3′
BIP
(SEQ ID NO: 3)
5′ - GGCGGCAGTCAAGCCTCTTCCCTACTGCTGCCTGGAGTT - 3′
F3
(SEQ ID NO: 4)
5′ - AGATCACATTGGCACCCG - 3′
B3
(SEQ ID NO: 5)
5′ - CCATTGCCAGCCATTCTAGC - 3′
LF
(SEQ ID NO: 6)
5′ - GCAATGTTGTTCCTTGAGGAAGTT - 3′

### Example 7

This example provides uses of systems of the present disclosure.

Highly accessible and highly accurate diagnostics are necessary to combat rapidly-spreading infectious diseases, such as the recent COVID-19 pandemic. While lateral flow antigen tests have become pervasive, they are insufficiently sensitive to detect early or asymptomatic disease. Nucleic acid amplification tests provide the needed sensitivity, but accessibility of these tests continues to be a challenge due to the need for precise sample processing steps. Reported herein is a sample-to-answer test for saliva samples (saliva-STAT) that utilizes a battery-powered handheld instrument and a low-cost easily-manufacturable sample cassette to perform a nucleic acid amplification test for viral pathogens. To enable a completely automated assay, thermally responsive alkane partitions (TRAPs) and paramagnetic beads were leveraged for virus purification and concentration, as well as reagent addition and mixing. Notably, the saliva STAT easily accommodates directly-dispensed saliva samples (in contrast to microfluidic devices), which is necessary for self-testing. Using the saliva-STAT platform, we demonstrate detection of down to 0.2 copies/μL of SARS-COV-2 virus in saliva samples. It is envisioned that the saliva-STAT could be used in walk-in clinics, mobile clinics, public testing locations, and in the home. With minor adjustments to the assay, the saliva-STAT platform can easily be adapted for other respiratory viruses, such as Influenza.

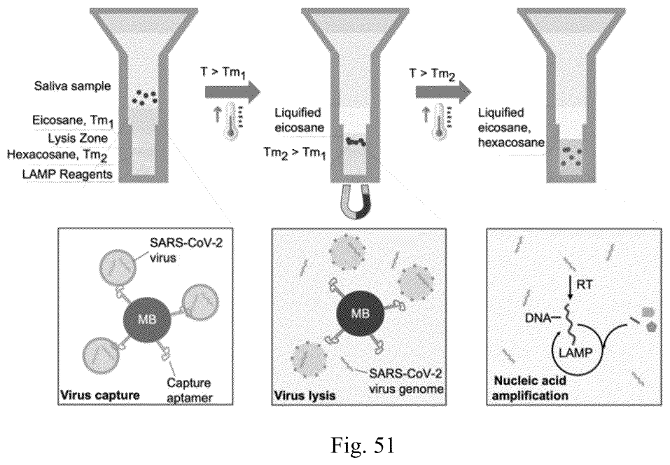

In this Example, sample-to-answer detection of viruses was demonstrated in large-volume saliva samples. The sample cassette and the assay are diagrammed in FIG. 51. The assay cassette is designed to accept a saliva sample directly from the user. As it is impossible for a user to precisely dispense sub-100 μL volumes of sample, the cassette must accept larger volumes, and the assay must be tolerant to variations in sample volume. After the sample is deposited in the cassette, aptamer-modified paramagnetic beads capture virus from the sample. Upon warming the cassette, the beads are pulled through a stationary TRAP into a protease-based lysis solution by a magnet positioned below the cassette. Following lysis, the cassette is further warmed to inactivate the protease and to melt a second TRAP, which releases reagents for loop mediated isothermal amplification (LAMP). The assay is controlled and analyzed by a handheld battery-powered instrument that provides temperature control, manipulates a magnet, and measures fluorescence. Using this sample-to-answer test for saliva samples (saliva-STAT), sensitive and specific detection of SARS-COV-2 virus in saliva down to 0.2 copies/μL in sample volumes as high as 500 μL was demonstrated.

Materials. The alkanes in this Example include n-eicosane (melting point ˜37° C.), 99%, and n-hexacosane (melting point ˜57° C.), 99%, both from ThermoFisher Scientific. Pierce Streptavidin Magnetic Beads (1.05 μm diameter) were purchased from ThermoFisher Scientific. Antarctic Thermolabile Uracil DNA Glycosylase (UDG) was purchased from NEB and is used throughout this work paired with DNA amplification reactions that result in nucleic acid products containing uracil bases. UDG helps to reduce the risk of carry-over contamination from previous DNA amplification reactions. WarmStart® Multi-Purpose LAMP/RT-LAMP Master Mix with UDG, LAMP Fluorescent Dye, and thermolabile Proteinase K were purchased from NEB. Gamma-irradiated SARS-COV-2 was supplied as a ˜10⁹ copies/mL stock solution from BEI Resources. All DNA sequences were purchased from IDT. DNA sequences are described below.

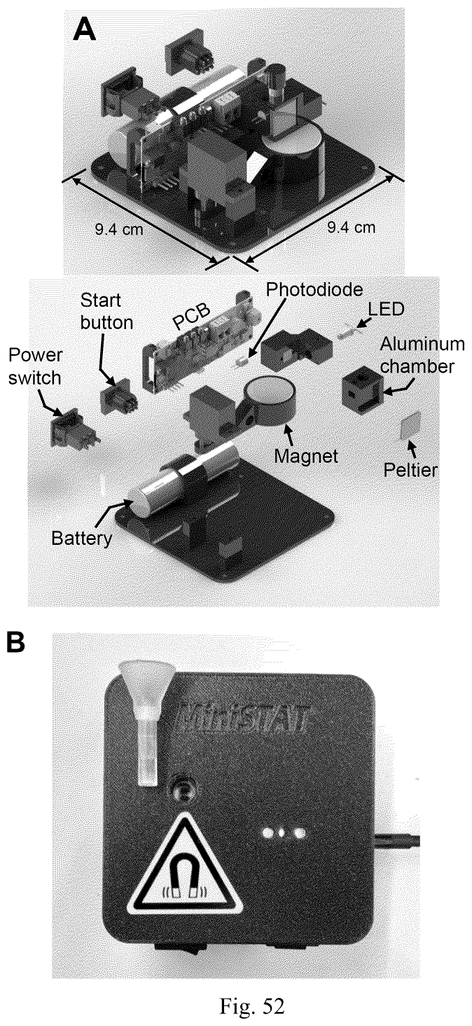

Saliva-STAT instrument. A battery-powered handheld instrument was designed and fabricated to control the assay and to measure fluorescence from the LAMP reaction. An exploded view of the instrument is shown in FIG. 52A. A bill of materials is included in the electronic supporting information. A photo of the instrument and the assay cassette is shown in FIG. 52B. The 3D-printed case has dimensions of 9.4×9.4×4 cm³. The instrument is composed of three subsystems: (1) a custom printed circuit board (PCB) with microcontroller (ATMEGA328P, Atmel), USB converter, heater controller, photodetector (FDS100, Thorlabs) and transimpedance amplifier, LED (470 nm), and analog-digital converter (AD4116, Analog Devices); (2) a custom-milled aluminum chamber that surrounds the cassette under test and holds a Peltier heater (NL1023T-01AC, Marlow Industries), digital temperature probe (DS18B20, Maxim Integrated), and optical bandpass filter (521 nm center wavelength, 20 nm full-width at half max, #26-717, Edmund Optics); and (3) a neodymium cylinder magnet 12.7 mm tall and 25.4 mm in diameter (DX08, K&J Magnetics) attached to a servo motor (SG92R, Adafruit) that automatically positions the magnet below or away from the assay cassette. Three indicator LEDs are also included on the PCB to indicate the instrument and assay status. The instrument is powered by a lithium polymer battery (ICR18650, Pkcell). An onboard charge controller (MCP73871, Microchip Inc.) and a boost converter (TPS61090, Texas Instruments) manage charging and power distribution to the rest of the system.

To aid with prototyping, a custom graphical user interface (GUI) was created in C # to operate the instrument and to display the temperature and fluorescence in real time. The assay can be initiated through the GUI or by pressing the “start” button on the instrument. The GUI connects to the microcontroller through USB. The microcontroller is programmed using the Arduino IDE programming software. The microcontroller utilizes a pulse-width modulation PID feedback loop to dynamically control the temperature of the assay cassette utilizing the Peltier heater and the temperature probe. The microcontroller cycles the temperature through the stages indicated in FIG. 51. For prototyping purposes, the stage timing and temperature can be controlled via the GUI. Additionally, the microcontroller turns on the excitation LED and collects the fluorescence emission readings from the analog-digital controller for the duration of the assay. Fluorescence values are sent through USB to the GUI for real time display. The microcontroller is also programmed to reposition the magnet using the servo motor. Initially the magnet is located below the assay cassette. After the paramagnetic beads are pulled through the first TRAP, the microcontroller actuates the servo motor to move the magnet away from the assay cassette.

Assay cassettes. Two types of cassettes were fabricated, the first by cutting via CNC an acrylic sheet with 24 mm thickness (McMaster-Carr) into 6.35 mm diameter cylinders, each with a channel 3.2 mm in diameter and 23 mm deep. The second type was fabricated by a similar method except the upper 14.5 mm of the channel was 4.6 mm in diameter. A funnel attachment was 3D printed via SLA using 3DM-Tough clear resin and the SL1S printer from Prusa. This funnel piece fits onto the opening of the larger volume cassette to allow for up to 500 μL of sample in the assay.

Paramagnetic bead manipulation in the saliva-STAT cassette. To quantify how well the magnet can gather beads to the alkane-saliva interface from a 500 μL sample of saliva, the following experiment was conducted. 25 μL of water was placed at the bottom of a larger volume cassette. 40 μL of eicosane was deposited on top of the water. Then, with the funnel attachment on the cassette, 500 μL of saliva with 100 μg paramagnetic beads was placed into the cassette on top of the eicosane layer. The cassette was placed onto a magnet and a video was recorded. The red color value of an area near the top of the funnel was extracted to show the movement of beads out of the saliva sample volume and toward the magnet.

Automatic reagent addition and mixing in the saliva-STAT. To demonstrate automatic reagent addition and rapid mixing, a DNA solution and an intercalating dye solution were partitioned by a thin TRAP in a cassette and then recorded the fluorescence as the temperature was increased. First, the bottom of a channel was loaded with 15 μL of DNA (a product of a previously run LAMP reaction, so the precise concentration of DNA is unknown). 8 μL of melted hexacosane was then placed on top of the DNA solution, where it solidified into a thin TRAP. Then, 10 μL 5×NEB LAMP Fluorescent Dye was loaded on top of the hardened hexacosane. To prevent evaporation, 40 μL eicosane was deposited on top to seal the solutions. The assembled cassette was then placed into the saliva-STAT instrument and fluorescence measurements were recorded as it heated up to 60° C. to melt the hexacosane.

Saliva-STAT LAMP Capabilities. To demonstrate the capability of the saliva-STAT instrument and cassette to rapidly amplify and detect genomic RNA, LAMP reactions with varying concentrations of SARS-COV-2 genome were run within the saliva-STAT instrument. First, 50 μL of 104 copies/μL SARS-COV-2 virus was incubated with 2.5 μL (0.3 units) of Proteinase K in phosphate buffered saline (PBS) for 15 minutes at 37° C., then 10 minutes at 55° C. to inactivate the Proteinase K. This solution was then diluted with PBS into several solutions of decreasing concentrations. Based in part on the recommended LAMP protocol from NEB, the 20 μL LAMP solution was prepared in nuclease-free water as follows: 1× WarmStart® Multi-Purpose LAMP/RT-LAMP Master Mix with UDG (NEB), 1×LAMP Fluorescent Dye (NEB), and LAMP primers (1.6 μM FIP/BIP, 0.2 μM F3/B3, and 0.4 μM LoopF/B) for the N15 gene. 5 μL of each of the lysed SARS-COV-2 solutions was added to LAMP reaction solutions. After vortexing, each sample was loaded into a cassette. The cassette was then loaded into the saliva-STAT instrument, which heated it at 65° C. for 60 minutes while recording the fluorescence of the solution. For the amplification curves, the time-to-positive (TTP) was calculated as the first timepoint for which the fluorescence value was 0.5% larger than the value one minute prior.

Detection of SARS-COV-2 in saliva samples with the saliva-STAT. To demonstrate virus detection in saliva samples using our sample-to-answer system, we tested SARS-COV-2 spiked samples with small volume (25 μL) samples and larger volume (500 μL) samples. First, 200 μg streptavidin paramagnetic beads were incubated with 50 pmol biotinylated capture aptamers for 15 minutes in 4 μL of PBS. The LAMP reaction solution was prepared by combining 25 μL WarmStart® Multi-Purpose LAMP/RT-LAMP 2× Master Mix with UDG (NEB), 1 μL 50×LAMP Fluorescent Dye (NEB), and 4 μL water. Lysis solution was prepared by combining 2 μL (˜0.25 units) Thermolabile Proteinase K, 5 μL LAMP primer mix containing 16 μM FIP/BIP, 2 μM F3/B3, and 4 μM LoopF/B for the N15 gene, 1 μL (1 unit) Antarctic Thermolabile UDG, and 12 μL water. 15 μL of the LAMP solution was added to the bottom of the acrylic cassette, followed by 8 μL melted hexacosane. Then 10 μL of the lysis solution was added after the hexacosane hardened, followed by 40 μL eicosane. 2 μL of the bead mixture was added after the eicosane hardened. 20 μL or 495 μL of saliva spiked with 5 μL of varying amounts of gamma-irradiated SARS-CoV-2 in PBS was added to each cassette. Concentrations of 200, 20, 2, 0.2, and 0 copies/μL in the 500 μL saliva samples as well as 2 and 0.2 copies/μL in the 25 μL saliva samples were prepared. The 500 μL samples were tested using the larger-volume assay cassette.

In the saliva-STAT, the cassette is subjected to 40° C. for 6 minutes to melt the first TRAP (eicosane); after it melts, the beads are automatically pulled through the eicosane by a magnet below the cassette. After 5.5 minutes, the magnet positioned below the cassette is automatically moved away via a rotating lever attached to a motor. At this point, the paramagnetic bead complexes are in the lysis solution, isolated from the saliva sample. The instrument then holds 37° C. for 6 minutes to allow Proteinase K to degrade any virus envelope proteins, compromising the stability of virus shells and releasing viral genome. The temperature then ramps up to 55° C. for 5 minutes to inactivate the Proteinase K. Finally, the temperature is increased to 65° C. to melt the second TRAP (hexacosane) to add the LAMP reagents to the lysate and initiate the LAMP reaction. This temperature is held for 60 minutes. Fluorescence measurements are recorded via the photodiode in the saliva-STAT. Conventional RT-qPCR comparison. RT-qPCR was performed on saliva

samples spiked with SARS-COV-2 to compare the detection capabilities of the assay against the gold standard nucleic acid diagnostic with a commercially available prep kit. 140 μL samples of saliva were spiked with varying concentrations of SARS-COV-2. The viral RNA was extracted into 60 μL elution reagent using a QIAamp Viral RNA Mini Kit (Qiagen) following the protocol provided in the QIAamp Viral RNA Mini Handbook. RT-qPCR reactions were assembled using a GoTaq® 1-Step RT-qPCR system (Promega), 1×SYBR Green 1, and 500 nM primers (nCOV_N1, IDT). 5 μL of extracted RNA was added to RT-qPCR reactions (20 μL total, N=5), and fluorescence was measured for 40 cycles in a CFX Opus 96 Real Time PCR system (BIO-RAD).

Paramagnetic bead isolation in a large-volume sample cassette. In previous work with stationary TRAPs, the magnet for pulling the beads through stationary TRAPs was located in close proximity to small-volume reagents containing the beads. For a fully automated saliva assay, it is necessary for the magnet to remain at the bottom of the cassette, but to pull the beads from a large volume saliva sample at the top of the cassette. In our cassette, the distance between the magnet in the instrument and the top of the eicosane TRAP (beginning of the inserted saliva sample layer) is about 10 mm, while the distance between the magnet and the top of the saliva sample layer (in the funnel attachment) is about 30 mm.

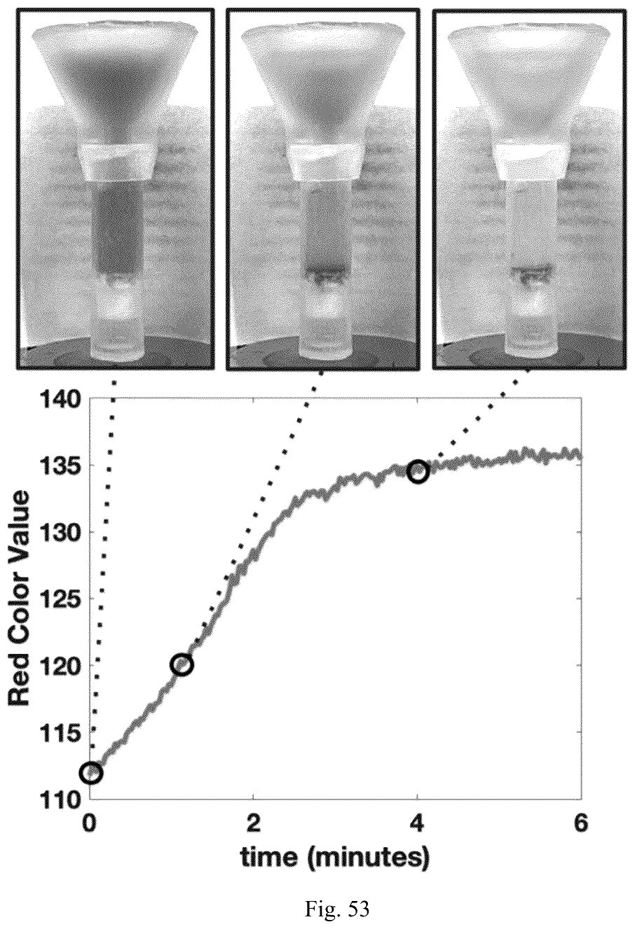

To quantify the speed with which the paramagnetic beads are pulled down in the cassette, a 500 μL sample containing paramagnetic beads was loaded into the assay cassette and placed the cassette on top of the magnet. A video of the beads migrating to the eicosane interface was recorded. The red color value was quantified in a region of interest near the top of the cassette. The results are presented in FIG. 53. The results demonstrate that after about three minutes, a sufficiently large majority of the beads are pulled from the sample to the sample-TRAP interface.

Automatic Reagent Addition and Mixing using TRAPs. Automatic reagent additions are necessary in point-of-care (PoC) devices that depend on reactions that must run in series. In the SARS-COV-2 saliva test, protease must lyse virus particles and then be deactivated before the solution is added to LAMP reagents. Otherwise, the protease will digest the polymerase and reverse transcriptase. Therefore, these reagents must be partitioned during the lysis step and automatically combined after the protease is deactivated. Breaching TRAPs integrated into a cassette enable this function.

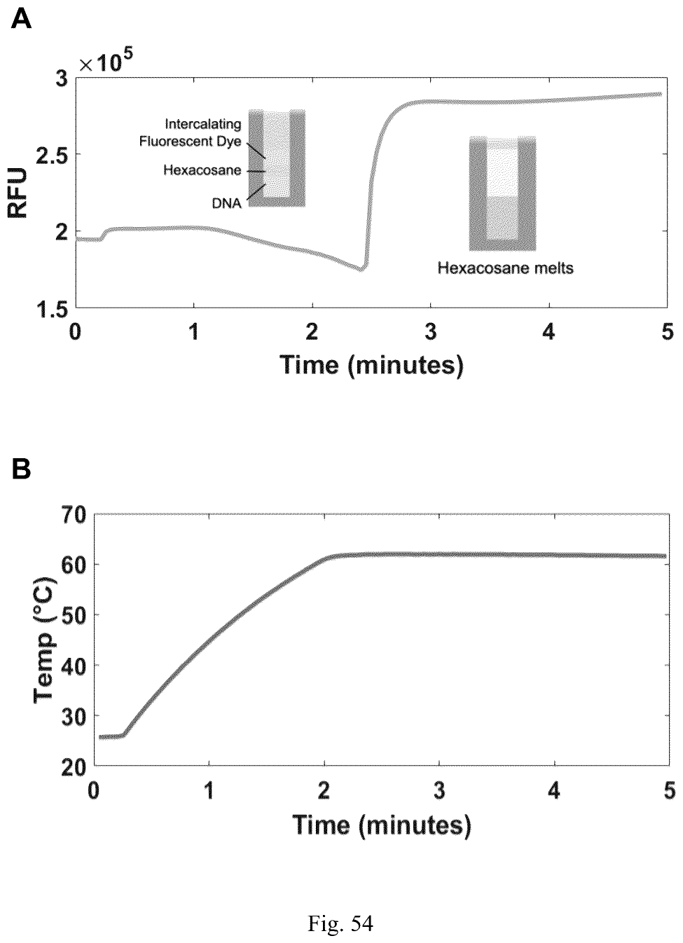

To demonstrate reagent partitioning and automated addition/mixing of reagents within the cassette, DNA and intercalating dye were loaded, partitioned by a thin hexacosane TRAP, into a cassette and placed the cassette into the instrument. Fluorescence near the bottom of the cassette was recorded while heat was applied to the cassette. The measured fluorescence and the temperature of the cassette are shown in FIG. 54. The fluorescence signal demonstrates that the DNA and intercalating dye remain partitioned until the temperature reaches the melt temperature of the TRAP. Once the TRAP melts, the DNA and dye are combined and the fluorescence signal immediately increases, demonstrating rapid mixing of the reagents. The reaction equilibrates within 1 minute of the breaching of the TRAP.

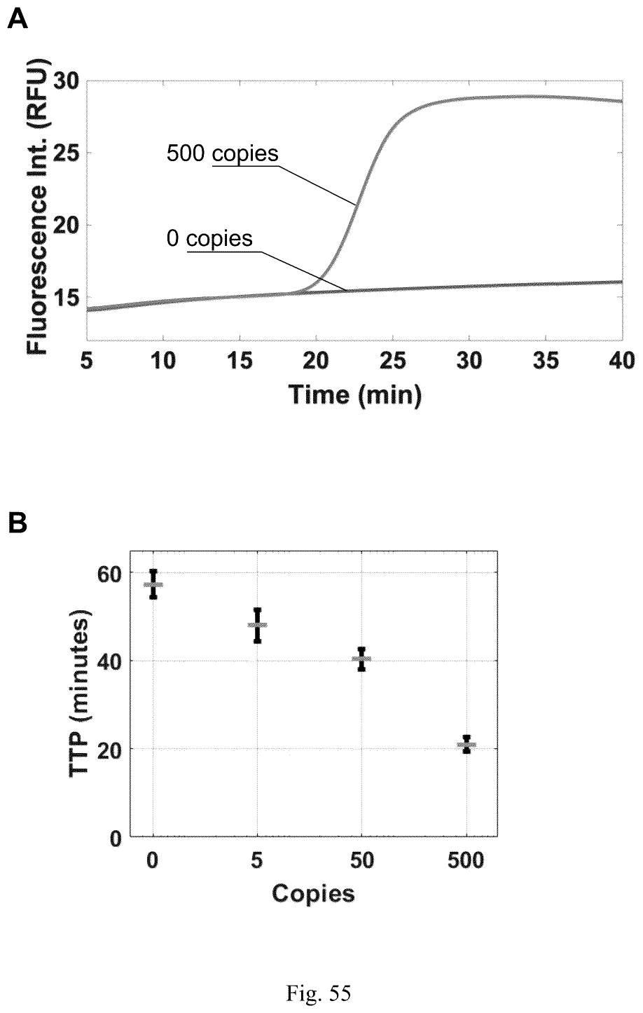

LAMP in the saliva-STAT. To demonstrate that the saliva-STAT instrument and cassette can reliably implement LAMP amplification of viral RNA, a range of concentrations of SARS-COV-2 RNA (i.e., after Proteinase K digestion of the virus) were added to LAMP master mix in saliva-STAT cassettes and capped the reaction volume with eicosane. FIG. 55A shows representative amplification curves for 500 copies and 0 copies, indicating that the instrument can easily measure the amplification signal and that LAMP is not inhibited by the cassette or the alkane. FIG. 55B presents the mean times-to-positive for 500, 50, 5, and 0 copies in the LAMP reaction in the saliva-STAT. The data demonstrates that 5 viral copies from a blank sample can reliably be differentiated.

Sample-to-answer detection of virus from large-volume saliva samples. In previously reported demonstrations of point-of-care saliva-based diagnostics, precisely measured small volumes of saliva were used. This is often required because the devices utilize microfluidics for sample processing. However, it is not practical to expect a user to dispense a precise, small volume of saliva. To address this challenge, the saliva STAT is designed to accommodate large sample volumes (500 μL). The combination of paramagnetic beads and the TRAPs enables the recovery of the virus particles from the large-volume sample and the automated transfer into the precisely pre-measured small-volume assay.

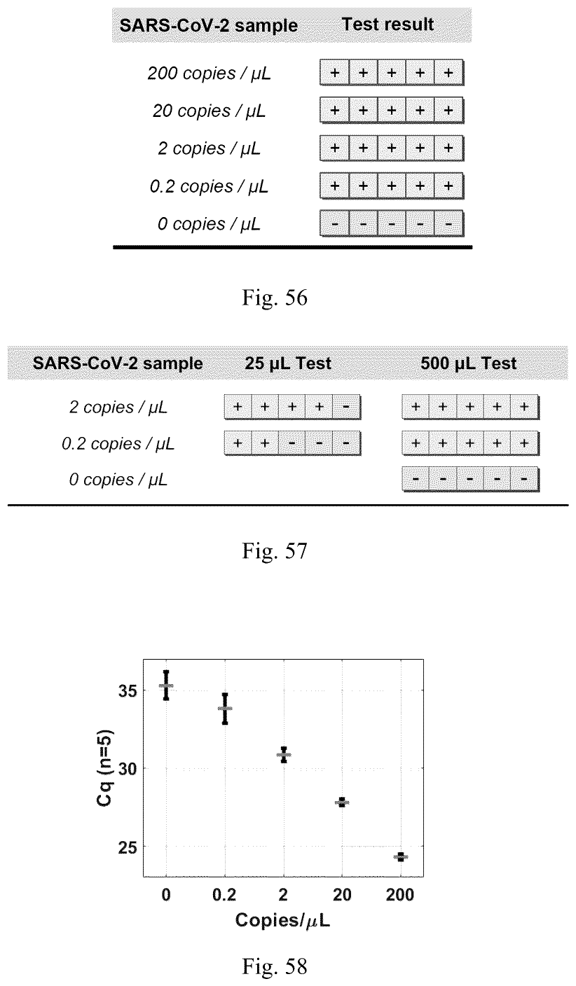

500 μL saliva samples spiked with SARS-COV-2 were tested at concentrations of 200, 20, 2, and 0.2 copies/μL (N=5). Samples that resulted in a time-to-positive (TTP) of less than 60 minutes from initiating the assay were considered positive for the virus. As shown in FIG. 56, all spiked samples were determined to be positive by the saliva-STAT, while all five samples that were not spiked with virus were determined to be negative by the saliva-STAT.

Accommodating larger volumes is advantageous for self-testing. Intuitively, it is also expected to have a performance advantage, as a larger sample volume will contain more viral copies. To illustrate this, 25 μL saliva samples spiked with SARS-COV-2 at concentrations of 2 and 0.2 copies/μL (N=5) were tested. For 2 copies/μL, 4/5 samples were correctly identified as positive, and for 0.2 copies/μL, only 2/5 samples were correctly identified as positive (FIG. 57). When using 500 μL samples, all samples at these concentrations were correctly identified as positive.

For further comparison, a standard RT-qPCR methodology for detecting SARS-COV-2 in saliva was performed. A commercially available Qiagen RNA recovery kit with a benchtop real-time PCR system was utilized. As with the saliva-STAT, down to 0.2 copies/μL from the blank sample were differentiated using this standard approach (FIG. 58). Using the Qiagen prep kit, 0.2 copies/μL leads to 2.3 RNA copies in the RT-qPCR reaction (assuming 100% RNA recovery), implying that this is near the theoretical limit of detection. As another point of comparison, it is noted that the SalivaDirect assay, which received Emergency Use Authorization from the US FDA, reports a limit of detection of 3 copies/μL. Thus, the saliva-STAT compares favorably with the standard benchtop RT-qPCR methodology while providing a portable sample-to-answer approach for testing saliva samples for viral disease.

In this Example, the sample-to-answer detection of SARS-COV-2 was demonstrated in saliva samples using a fully automated, user-friendly, battery-powered handheld device and a low-cost, easily manufacturable sample cassette. Currently, point-of-care saliva-based nucleic acid amplification tests for respiratory pathogens are not available because of the challenges of automating all of the assay steps in a low-cost and portable system. A magnetofluidic assay with TRAPs was leveraged to enable (i) the recovery of virus particles from the large-volume saliva sample, (ii) the addition of the virus to lysis reagent, and (iii) the addition of the released RNA to the LAMP reagents. All of these steps were accomplished with a handheld instrument and an easily manufacturable assay cassette. The saliva STAT correctly identified 20/20 spiked samples as positive, including five samples with 0.2 copies/μL of SARS-COV-2 virus, while also correctly identifying five negative samples. These perfectly sensitive and specific results were achieved with an 80-minute assay time (including automated sample preparation), though only two positive samples required more than 50 minutes of total assay time. The value of a platform that is capable of testing large-volume samples was also demonstrated, as it was shown that the sensitivity of testing 500 μL samples was significantly better than when testing 25 μL samples.

The application of the saliva-STAT can be applied to other respiratory viruses that can be detected in saliva. One only needs to change the capture aptamer on the paramagnetic beads and the LAMP primers to adapt the saliva-STAT to other viruses. In addition, an additional optical channel can be added to the instrument to enable the simultaneous screening of multiple viruses, such as SARS-COV-2 and influenza H5N1 (i.e., avian influenza).

DNA sequences. Virus particles were captured onto streptavidin-functionalized paramagnetic beads using biotinylated SARS-COV-2 RBD aptamer:

(SEQ ID NO: 1)
5′-ATCCAGAGTGACGCAGCATTTCATCGGGTCCAAAAGGGGCTGCTCGG
GATTGCGGATATGGACACGTTTTTTTT/3Bio/ - 3′.

The SARS-COV-2 RNA genome was amplified with LAMP using the following primers for the N15 gene.

(SEQ ID NO: 2)
5′ - TGCTCCCTTCTGCGTAGAAGCCAATGCTGCAATCGTGCTAC -
3′
BIP
(SEQ ID NO: 3)
5′ - GGCGGCAGTCAAGCCTCTTCCCTACTGCTGCCTGGAGTT - 3′
F3
(SEQ ID NO: 4)
5′ - AGATCACATTGGCACCCG - 3′
B3
(SEQ ID NO: 5)
5′ - CCATTGCCAGCCATTCTAGC - 3′
LF
(SEQ ID NO: 6)
5′ - GCAATGTTGTTCCTTGAGGAAGTT - 3′
LB
(SEQ ID NO: 7)
5′ - GTTCCTCATCACGTAGTCGCAACA - 3′

Bill of materials for saliva-STAT instrument. Listed below are all components in the saliva-STAT instrument, along with prices. Prices are specified for a quantity of five builds. It is noted that the majority of the cost is for the PCB and the milled aluminum heat block. Prices for these items would fall drastically for large order. It is estimated that a run of 1000 instruments would have a per-build cost of approximately $100.

Saliva-STAT Bill of Materials
quantify of 5

| Part Description | Manufacturer | Manufacturer's Part Number | Source | Unit Cost | Quantity | Total Cost |
|---|---|---|---|---|---|---|
| Control PCB (custom) | UMD | ministat_rev0 | Macrofab | 367.42 | 1 | $ 367.42 |
| Heating Chamber (custom) | Xometry | stat_chamber_rev1 | Xometry | 150.24 | 1 | $ 150.24 |
| Photodiode | Thorlabs | FDS100-P5 | Thorlabs | 15.86 | 1 | $ 15.86 |
| Peltier Junction | Marlow Industries | NL1023T-01AC | Digikey | 38.89 | 1 | $ 38.89 |
| Digital temperature sensor | Dallas Semiconductor | DS18B20 | Digikey | 3.95 | 1 | $ 3.95 |
| 465 nm LED | CO RODE | B00UWBJM0Q | Amazon | 0.03 | 1 | $ 0.03 |
| 521 nm OD8 Emission filter | Edmund Optics | 26-717 | Edmund Optics | 190 | 1 | $ 190.00 |
| Power Switch | TE Connectivity | 1825421-1 | Digikey | 21.96 | 1 | $ 21.96 |
| Start Button | E-switch | 700SP7M1REAP2BLKBLK | Digikey | 4.58 | 1 | $ 4.58 |
| 2200maH Lipo battery | Adafruit Industries | 1781 | Digikey | 9.95 | 1 | $ 9.95 |
| 44 lb pull Neomydium magnet | K&J Magnetics | DX08 | K&J Magnetics | 10.32 | 1 | $ 10.32 |
| Servo motor | Adafruit Industries | 169 | Digikey | 5.95 | 1 | $ 5.95 |
| Brass 2-56 × ¼″ flathead screws | Mcmaster | 92480A863 | Mcmaster | 0.0731 | 16 | $ 1.17 |
| 3D printed Housing (custom) | UMD | ministat_CAD_rev0 | UMD | 1 | 1 | $ 1.00 |
| | | | | | Total: | $ 821.32 |

FIG. 18 illustrates the assembly of the components within the custom instrument. CAD files for the custom parts will be provided upon request.

Detection comparison between small and large volume. FIG. 57 shows that the detection performance improves with the larger sample volume. This is because the saliva-STAT system is able to capture viral copies from the sample and transfer them into the small-volume RT-LAMP reaction. Larger sample volumes have more viral copies, thus provide more RNA copies for the RT-LAMP reaction.

Conventional RT-qPCR comparison. FIG. 58 shows results from RT-qPCR on saliva samples using a Qiagen RNA prep kit and commercial benchtop real-time PCR system. Similar to the saliva-STAT, it was able to detect down to 0.2 copies/μL in saliva.

Although the present disclosure has been described with respect to one or more particular embodiments and/or examples, it will be understood that other embodiments and/or examples of the present disclosure may be made without departing from the scope of the present disclosure.

1. A method for detecting the presence of viral analyte comprising:
i) contacting a sample suspected of containing the viral analyte with a plurality of magnetic beads having a plurality of capture molecules attached thereto, wherein the capture molecules have a specific affinity for the analyte to form complexes, each complex comprising the analyte bound to the capture molecule;
ii) heating a first solid aliphatic partitions to a temperature of 35° C. to 45° C., such that the first solid aliphatic partition liquefies;
iii) selectively moving the complexes through the liquefied aliphatic partition via application of a magnetic field;
iv) contacting the complex with a protease such that viral genomic material is released from the viral analyte;
v) heating the complex, the protease, and a second solid aliphatic partition to a temperature greater than or equal to 50° C., such that the protease deactivates and the second aliphatic partition liquefies;
vi) contacting the viral genomic material with genomic amplification reagents;
vii) amplifying the viral genomic material;
viii) contacting the amplified viral genomic material with a detectable molecule; and
ix) detecting and optionally quantifying a signal generated from the detectable molecule,
wherein the presence of a detectable signal is indicative of the presence of the viral analyte and the magnitude of the signal is indicative of the amount of the viral analyte in the sample.

2. The method of claim 1, wherein the sample is whole blood, blood fractions, plasma, serum, saliva, urine, stool, sweat, mucous, tears, breast milk, semen, tissue, placental tissue, conditioned medium, tissue culture medium, or bone marrow.

3. The method of claim 2, wherein the sample is whole blood.

4. The method of claim 1, wherein the viral analyte is a viral particle.

5. The method of claim 1, wherein the protease is Proteinase K.

6. The method of claim 1, wherein the first aliphatic partition is eicosane.

7. The method of claim 1, wherein the second aliphatic partition is hexacosane.

8. The method of claim 1, wherein the viral analyte is associated with human immunodeficiency virus, SARS-COV-2, Hepatitis C, Epstein-Barr, Zika, Ebola, Herpes simplex, Norovirus, Influenza, or Chikungunya.

9. The method of claim, wherein when the second aliphatic partition liquefies, it results in addition of genomic amplification reagents to a mixture of the complex and viral genomic material.

10. A point-of-care testing device, comprising:
a chamber configured to receive a sample cartridge;
a heater configured to heat the sample cartridge;
an optical detector configured to detect an optical property of at least a portion of a material within the sample cartridge; and
a processor in electronic communication with the heater and the optical detector, the processor having a memory for storing a testing profile, and wherein the processor is configured to:
operate the heater to heat the sample cartridge according to a testing profile; and
receive a measurement signal from the optical detector at a predetermined time of the profile.

11. The device of claim 10, wherein the optical detector is configured to detect fluorescence.

12. The device of claim 10, further comprising a sample cartridge, the sample cartridge including a first reagent and a second reagent, where the first reagent and the second reagent are separated by a first meltable solid partition, such as a solid aliphatic partition.

13. The device of claim 12, wherein the sample cartridge is configured to receive a sample from a user and wherein the sample is in contact with the first reagent.

14. The device of claim 12, wherein the processor is configured to heat the sample cartridge to a temperature sufficient to liquefy the first meltable solid partition at a predetermined time of the testing profile.

15. The device of claim 12, wherein the meltable solid partition is a solid aliphatic partition.

16. The device of claim 10, further comprising:
a magnet disposed adjacent to the chamber; and
an actuator for moving the magnet from a first location to a second location.

17. The device of claim 12, wherein the sample cartridge further comprises a third reagent separated from the second partition by a second meltable solid partition.

18. The device of claim 12, wherein the testing profile of the processor is configured to:
heat the sample cartridge to liquefy the first meltable partition and allow the magnetic microbeads to pass through the first meltable partition;
stop heating the sample cartridge for a predetermined period of time;
heat the sample cartridge to a second temperature to liquefy the second meltable partition and allow the second reagent and third reagent to mix; and
receive a measurement signal from the optical detector at a predetermined time of the testing profile.

19. The device of claim 10, further comprising a user input and the processor is further configured to begin the testing profile upon receiving a first signal from the user input.

20. The device of claim 10, further comprising an indicator and the processor is further configured to provide a result signal to the indicator based on the received measurement signal.


SEQUENCE LISTING (7 sequences)

SEQ ID NO: 1 - DNA, 74 bp, synthetic construct
  source 1..74: mol_type = other DNA
atccagagtgacgcagcatttcatcgggtccaaaaggggctgctcgggattgcggatatggacacgtttttttt

SEQ ID NO: 2 - DNA, 41 bp, synthetic construct
  source 1..41: mol_type = other DNA
tgctcccttctgcgtagaagccaatgctgcaatcgtgctac

SEQ ID NO: 3 - DNA, 39 bp, synthetic construct
  source 1..39: mol_type = other DNA
ggcggcagtcaagcctcttccctactgctgcctggagtt

SEQ ID NO: 4 - DNA, 18 bp, synthetic construct
  source 1..18: mol_type = other DNA
agatcacattggcacccg

SEQ ID NO: 5 - DNA, 20 bp, synthetic construct
  source 1..20: mol_type = other DNA
ccattgccagccattctagc

SEQ ID NO: 6 - DNA, 24 bp, synthetic construct
  source 1..24: mol_type = other DNA
gcaatgttgttccttgaggaagtt

SEQ ID NO: 7 - DNA, 24 bp, synthetic construct
  source 1..24: mol_type = other DNA
gttcctcatcacgtagtcgcaaca